(19) United States
(12) Patent Application Publication
(10) Pub. No.: US 2025/0313894 A1
(43) Pub. Date: Oct. 9, 2025

(54) ARRAY OF ASTHMA- AND ALLERGY-ASSOCIATED DIFFERENTIALLY-METHYLATED SITES

(71) Applicant: The University of Chicago, Chicago, IL (US)

(72) Inventors: Carole Ober, Chicago, IL (US); Andreanne Morin, Chicago, IL (US); Britney Helling, Chicago, IL (US); Emma Thompson, Chicago, IL (US)

(21) Appl. No.: 18/865,031

(22) PCT Filed: May 16, 2023
(86) PCT No.: PCT/US2023/067047

§ 371 Date: Nov. 12, 2024

Related U.S. Application Data
(60) Provisional application No. 63/342,463, filed on May 16, 2022.
(60) Provisional application No. 63/502,195, filed on May 15, 2023.

(51) Int. Cl. C12Q 1/6883

(52) U.S. Cl. CPC C12Q 1/6883; C12Q 2600/154

(57) ABSTRACT

Provided herein are DNA methylation arrays displaying oligonucleotides containing human CpG sites that are differentially methylated in subjects suffering from asthma and/or allergies relative to the general population, and methods of use thereof.

### STATEMENT REGARDING RELATED APPLICATIONS

This application claims priority to U.S. Provisional Patent Application No. 63/342,463, filed May 16, 2022, and to U.S. Provisional Patent Application No. 63/502,195, filed May 15, 2023, the entire contents of which are incorporated herein by reference for all purposes.

### STATEMENT REGARDING FEDERAL FUNDING

This invention was made with government support under grant number OD023282 awarded by National Institutes of Health. The government has certain rights in the invention.

### TABLE

The specification of U.S. Provisional Patent Application No. 63/502,195 includes a lengthy table, Table A, which was submitted via EFS-Web in electronic format as follows: File name: TableA_targetCpGs.txt, Date created: May 15, 2023, File size: 582,259 Bytes. The content of Table A is hereby incorporated by reference in its entirety.

### SEQUENCE LISTING

The computer readable sequence listing filed herewith, titled “UCHI-39941-601_SQL”, created May 13, 2023, having a file size of 49,251,874 bytes, is hereby incorporated by reference in its entirety.

### FIELD

Provided herein are DNA methylation arrays displaying oligonucleotides containing human CpG sites that are differentially methylated in subjects suffering from asthma and/or allergic disease relative to the general population, and methods of use thereof.

### BACKGROUND

Epigenetics refers to modifications of DNA molecules that do not alter the DNA sequence but play important roles in regulating gene expression. Environmental exposures can directly modify epigenetic marks in the human genome and epigenetic responses can mediate the effects of exposures on gene expression and disease risk. Thus, the epigenome may contribute directly to disease risk or be sites of gene-environment interactions, providing both complementary and mechanistic information, respectively, to genome-wide association studies (GWAS). The most common epigenetic mark in the human genome is methylated cytosines at CpG dinucleotides, and the availability of high-throughput array-based platforms to measure DNA methylation has led to an explosion of epigenome-wide association studies. However, although the most commonly used commercial array, the Infinity Methylation EPIC Beadchip (Illumina, Inc., San Diego, CA), interrogates up to 850,000 CpGs, this represents <5% of CpGs in the genome. Moreover, the selection of CpGs for this array was agnostic with respect to disease or tissue types. Accordingly, what is needed are arrays to detect CpG sites that contribute to disease risk, including asthma and allergy.

### SUMMARY

Provided herein are DNA methylation arrays displaying oligonucleotides containing human CpG sites that are differentially methylated in subjects suffering from asthma and/or allergic disease relative to the general population, and methods of use thereof.

In some embodiments, the arrays described herein are used to detect the methylation of genomic DNA from a human subject.

In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each probe oligonucleotide comprising a distinct sequence capable of hybridizing to a human genomic location identified in Table A.

In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides comprising a distinct sequence that is complementary to a human genomic location identified in Table A.

In some embodiments, a probe oligonucleotide comprises a portion (e.g., 10⁻⁵⁰ nucleotides (e.g., 10, 15, 20, 25, 30, 35, 40, 45, 50, or ranges therebetween) of a sequence complementary to a human genomic location identified in Table A, and terminating at a methylation site within the sequence.

In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each probe oligonucleotide comprising a distinct sequence having at least 90% sequence identity to (e.g. at least 90%, at least 91%, at least 92%, at least 93%, at least 94%, at least 95%, at least 96%, at least 97%, at least 98%, at least 99%, or 100% identity to) SEQ ID NO: 1-SEQ ID NO: 53,840. In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each probe oligonucleotide comprising a distinct sequence selected from SEQ ID NO: 1-SEQ ID NO: 53,840.

In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) type I probe oligonucleotides. A type I probe oligonucleotide refers to a probe oligonucleotide wherein a single probe oligonucleotide is used to detect a target. In contrast, a type II probe oligonucleotide refers to a probe oligonucleotide wherein two probe oligonucleotides are used to detect a target. SEQ ID NO: 1-SEQ ID NO: 37942 correspond to type I probe oligonucleotides. In some embodiments, provided herein are compositions comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) type II probe oligonucleotides. SEQ ID NO: 37,943-SEQ ID NO: 53,840 correspond to type II probe oligonucleotides. In some embodiments, the composition comprises type I and type II probe oligonucleotides. In some embodiments, the composition comprises type II probe oligonucleotide pairs. A type II probe oligonucleotide pair refers to the two probe oligonucleotides used to detect a given target. Type II probe oligonucleotide pairs are exemplified by two sequential sequences within SEQ ID NO: 37,943-SEQ ID NO: 53,840, starting with a first pair shown in SEQ ID NO: 37,493 and SEQ ID NO: 37,494, a second pair shown in SEQ ID NO: 37,495 and SEQ ID NO: 37,496, a third pair shown in SEQ ID NO: 37,497 and SEQ ID NO: 37,498, and so on.

In some embodiments, the probe oligonucleotide corresponds to the unmethylated methylation site and terminates in a 3′ CA (complementary to a CpG site modified by bisulfite treatment and amplification). In some embodiments, such a probe oligonucleotide is capable of hybridizing to a sample nucleic acid corresponding to a methylated or unmethylated site (e.g., a differentially-modified oligonucleotide generated from a methylated or unmethylated site) but only allowing single nucleotide extension from a sample nucleic acid corresponding to the unmethylated methylation site.

In some embodiments, the probe oligonucleotide corresponds to the unmethylated methylation site and terminates in a 3′ CG (complementary to a CpG site unmodified by bisulfite treatment and amplification). In some embodiments, such a probe oligonucleotide is capable of hybridizing to a sample nucleic acid corresponding to a methylated or unmethylated site (e.g., a differentially-modified oligonucleotide generated from a methylated or unmethylated site) but only allowing single nucleotide extension from a sample nucleic acid corresponding to the methylated methylation site.

In some embodiments, the probe oligonucleotide corresponds to a methylation site and terminates in a 3′ C (complementary to the G of a CpG site). In some embodiments, such a probe oligonucleotide is capable of hybridizing to a sample nucleic acid corresponding to a methylated or unmethylated site (e.g., a differentially-modified oligonucleotide generated from a methylated or unmethylated site) and allowing single nucleotide extension from a sample nucleic acid corresponding to either the methylated or unmethylated methylation site.

In some embodiments, probe oligonucleotides herein comprise a linker oligonucleotide (e.g., 2-25 nucleotides in length) at the 5′ end of the probe. In some embodiments, the 5′ end of the probe oligonucleotide tenrinates in a functional group capable of attachment to a solid surface.

In some embodiments, probe oligonucleotides are deoxyribonucleic acid (DNA) oligonucleotides.

In some embodiments, provided herein are devices (e.g., arrays) comprising the composition of 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) of the probe oligonucleotides described herein, wherein the probe oligonucleotides are displayed on a surface of a substrate. (e.g., a solid surface). In some embodiments, provided herein are devices (e.g. arrays) comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each probe oligonucleotide comprising a distinct sequence having at least 90% sequence identity to (e.g. at least 90%, at least 91%, at least 92%, at least 93%, at least 94%, at least 95%, at least 96%, at least 97%, at least 98%, at least 99%, or 100% identity to) SEQ ID NO: 1-SEQ ID NO: 53,840. In some embodiments, provided herein are devices (e.g. arrays) comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each probe oligonucleotide comprising a distinct sequence selected from SEQ ID NO: 1-SEQ ID NO: 53,840. In some embodiments, the probe oligonucleotides are displayed on the surface of a substrate (e.g. a solid surface). In some embodiments, the array comprises type I and/or type II oligonucleotides, as described herein. In some embodiments, the substrate is selected from a bead, a slide, a plate, well, etc. In some embodiments, the surface comprises plastic, glass, metal, etc. In some embodiments, the oligonucleotides are tethered to the surface of the substrate. In some embodiments, the surface is coated with a material to allow attachment of the probe oligonucleotides. In some embodiments, the substrate comprises one or more array locations and the oligonucleotides are displayed on a surface within the array location. In some embodiments, the substrate is a microtiter plate and the array locations are microtiter wells. In some embodiments, each array location comprises a plurality of discrete sites for attachment of oligonucleotides to the substrate. In some embodiments, each discrete site is a bead well within the surface of the substrate. In some embodiments, the oligonucleotides are tethered to beads and the beads reside within the bead wells on the surface of the substrate. In some embodiments, each of the probe oligonucleotides are tethered to a separate bead. In some embodiments, each of the array locations comprises at least 1,000 discrete sites per cm² (e.g., >1,000 sites/cm², >2,000 sites/cm², >5,000 sites/cm², >10,000 sites/cm², >20,000 sites/cm², >50,000 sites/cm², >100,000 sites/cm², >200,000 sites/cm², >500,000 sites/cm², or >1,000,000 sites/cm²).

In some embodiments, provided herein are methods of detecting the presence of nucleic acid sequences in a sample, comprising: (a) contacting probe oligonucleotides described herein (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) or a device displaying such probe oligonucleotides with a nucleic acid sample; and (b) detecting the binding of one or more nucleic acids comprising the nucleic acid sequences to one or more of the probe oligonucleotides.

In some embodiments, provided herein are methods of detecting the methylation status of methylation sites in a nucleic acid in a sample, the method comprising: (a) treating the sample to differentially modify the nucleic acid at methylated and unmethylated methylation sites to produce a differentially-modified nucleic acid; (b) amplifying the differentially-modified nucleic acid; (c) fragmenting the differentially-modified nucleic acid into differentially-modified oligonucleotides; (d) contacting a device described herein with the differentially-modified oligonucleotides, and allowing the differentially-modified oligonucleotides to hybridize to the probe oligonucleotides, thereby forming probe/differentially-modified oligonucleotide complexes; (e) labeling the probe/differentially-modified oligonucleotide complexes in a manner that is specific to whether the differentially-modified oligonucleotide of each complex corresponds to a methylated or unmethylated methylation site; (f) detecting the labeled probe/differentially-modified oligonucleotide complexes; and (g) analyzing (1) the type of labeling and (2) the location of the probe/differentially-modified oligonucleotide complexes on the surface.

In some embodiments, amplifying the differentially-modified nucleic acid comprises PCR amplification.

In some embodiments, treating the sample to differentially modify the nucleic acid at methylated and unmethylated methylation sites comprises exposing the sample to bisulfite that converts unmethylated cytosine to uracil but methylated cytosines are protected from conversion. In some embodiments, amplifying the differentially-modified nucleic acid converts the uracil generated by bisulfite conversion into thymine. In some embodiments, treating the sample to differentially modify the nucleic acid at methylated and unmethylated methylation sites comprises exposing the sample to bisulfite that converts unmethylated cytosine to uracil but methylated cytosines are protected from conversion.

In some embodiments, fragmenting the differentially-modified nucleic acid comprises site-specific fragmentation of the differentially-modified nucleic acid. In some embodiments, site-specific fragmentation is by restriction endonuclease. In some embodiments, fragmenting the differentially-modified nucleic acid comprises random fragmentation of the differentially-modified nucleic acid. In some embodiments, random fragmentation comprises chemical, enzymatic, and/or mechanical fragmentation.

In some embodiments, methods further comprise a step of isolating the differentially-modified nucleic acid and/or differentially-modified oligonucleotides from reagents for amplification and/or fragmentation.

In some embodiments, labeling the probe/differentially-modified oligonucleotide complexes in a manner that is specific to whether the differentially-modified oligonucleotide of each complex comprises performing a single nucleotide extension reaction with labeled nucleotides. In some embodiments, the labeled nucleotides comprise happens. In some embodiments, methods further comprise contacting probe/differentially-modified oligonucleotide complexes following the single nucleotide extension with antibodies capable of binding to the haptens, wherein the antibodies comprise detectable labels. In some embodiments, the labeled nucleotides comprise detectable labels. In some embodiments, the detectable labels comprise fluorescent labels.

In some embodiments, nucleic acid samples comprising genomic DNA. In some embodiments, the genomic DNA is human genomic DNA. In some embodiments, the human genomic DNA is obtained from airway epithelial cells. In some embodiments, the cells are obtained from a subject suffering from asthma and/or allergic disease. In some embodiments, the cells are obtained from a subject suspected as having asthma and/or allergic disease.

### BRIEF DESCRIPTION OF THE DRAWINGS

FIGS. 1A-ID show an overview of exemplary methods described herein. (FIG. 1A) Whole-genome bisulfite sequencing (WGBS) and differential methylation were performed in airway epithelial cell DNA from birth cohorts comprised of African American children or European American young adults (half of each with allergic asthma and half without asthma or allergies). To select CpGs for the Custom array, CpGs were identified based on prior evidence of association with asthma or allergic disease from three sources. The CpGs were then prioritized based on their overlap with functional annotations to ultimately design a Custom array with 53,840 probes for 45,891 CpGs. (FIG. 1B) Airway epithelial cell DNA was hybridized to both the Custom array and the EPIC array, and β value distributions on the arrays were compared to the WGBS data and across tissues using the Custom and EPIC array. (FIG. 1C, FIG. 1D) The Custom array was validated by performing an EWAS of allergic sensitization (AS), examining correlations with the gene expression in the same cells, and replicating findings in a second cohort and with allergic asthma. (FIG. 1E) The combined results of the Custom and EPIC arrays in the discovery cohort and of the Custom array in the replication cohort for known and novel loci were examined in regional association plots.

FIGS. 2A-2D show proportions of Custom and EPIC CpGs by primary criteria, functional annotation category, and genomic location. All comparisons were performed with a Fisher's Exact Test. (FIG. 2A) Proportions of Custom CpGs that passed processing QC in URECA on each array by three primary criteria and six functional annotation categories. CpGs on the Custom array were significantly enriched (p<2.2×10⁻¹⁶) in all categories compared to the EPIC array with the exception of prior EWAS, in which they were depleted (p<2.2×10⁻¹⁶). (FIG. 2B) The distributions of CpGs by genomic location. Compared to the EPIC CpGs, CpGs on the Custom array were enriched in introns and exons and depleted in intergenic regions and 5′UTRs (p<10⁻⁴), but did not differ in any other categories. (FIG. 2C) Proportions of Custom and EPIC DMCs from each array were compared by three primary criteria and six functional annotation categories. Compared to all CpGs, DMCs from both arrays were depleted at transcription start sites and in areas of open chromatin (Custom p=1.14×10⁻⁷ and p=4.11×10⁻⁹; EPIC p<2.2×10⁻¹⁶ for both, respectively). DMRs were marginally enriched for DMCs compared to all CpGs on the Custom array (p=0.066) but significantly enriched on the EPIC array (p=1.54×10⁻⁶). DMCs in prior EWAS studies of asthma and allergic diseases were modestly enriched on the Custom array (FET p=0.059) and significantly enriched on the EPIC array (p<2.2×10⁻¹⁶). In contrast, CpGs at GWAS loci for asthma and allergic diseases were not enriched among DMCs on the Custom array (p=0.32) and only modestly enriched on the EPIC array (p=0.042). The distribution of DMCs by genomic location is shown in FIG. D. The distribution of DMCs on both arrays did not differ from the distributions of all CpGs.

FIGS. 3A-3B show cross-tissue comparisons of methylation levels on the EPIC and Custom arrays. Density distribution plots of methylation levels, measured as β values, in 96 individuals after quantile (Custom) or quantile+SWAN (EPIC) normalizations. The x-axis shows the proportion of methylation at CpG sites on the EPIC (FIG. 3A) and Custom (FIG. 3B) arrays for each individual in each cell or tissue type. All DNA methylation data were processed using the same pipeline. The nasal epithelial cell DNA (EPIC and Custom) and nasal lavage cell DNA (Custom) were from the same randomly selected 96 URECA children with both the EPIC and Custom array in nasal epithelial cells. The buccal, placenta, and cord blood cells were from infants in the VCSIP Study.

FIGS. 4A-4G show allergic sensitization (AS) EWAS results in URECA and INSPIRE children using the EPIC and Custom arrays. (FIG. 4A-4C) Upper panel: Volcano plots showing the log 2-fold change in methylation (M-values) by proportion positive skin prick tests (x-axis) and the −log₁₀(P-value) from the EWAS (y-axis). Significant DMCs at a q-value threshold of 0.05 are shown are shown in blue or purple (EPIC and high-value EPIC, respectively) in URECA (FIG. 4A), and Custom in red (URECA) (FIG. 4B) or turquoise (INSPIRE) (FIG. 4C). The number of AS DMCs that were hypermethylated or hypomethylated are shown as up and down arrows, respectively. (FIG. 4A-4C) Middle panel: Density plots of the DMC β values in each EWAS. (FIG. 4A-4B) Lower panel: Density plots of 0 values for DMCs that are eQTMs for their nearest genes. (FIG. 4D) Distributions of the distances of DMCs to the next nearest DMC on the Custom (red) and EPIC (blue) arrays. The distance to nearest DMC is shown on the x-axis; the proportion of DMCs in each distance bin is shown on the y-axis. (FIG. 4E-4F) Correlation of effect sizes (log fold change) of AS DMCs and allergic asthma in URECA and INSPIRE. The effect size of DMCs for AS (x-axis) and allergic asthma (y-axis) are shown. Red or turquoise dots were associated with allergic asthma at a q-value ≤0.05 in URECA or INSPIRE, respectively; black dots are AS-only DMCs. (FIG. 4G) Correlation of effect sizes (log fold change) between DMCs identified in the INSPIRE AS EWAS (x-axis) and the URECA AS EWAS (y-axis). Red or turquoise points are AS DMCs only in URECA or INSPIRE, respectively, at a q-value threshold of 0.05; black points are AS DMCs in both at a q-value ≤0.05. The dashed lines in E-G are the correlation lines.

FIG. 5 shows an exemplary functional genomics pipeline for identifying high-value CpGs. The pipeline for selecting high-value CpGs involved three steps. In Step 1, all CpGs with some prior evidence linking them to asthma or allergic diseases were identified in three categories. Using WGBS data, CpG sites within asthma DMRs in ethnically diverse children were identified (red box). Based on literature reviews, CpGs associated with asthma or allergic diseases in other DNA methylation studies (blue box) and CpGs at asthma or allergic disease GWAS loci (green box) were identified. After removing duplicates, CpGs that are on the EPIC array, in blacklisted regions of the genome, or overlapped with common SNPs were removed. In Step 2, the CpGs were overlapped with functional annotations and required that CpGs in DMRs from the WGBS overlapped with at least three annotations (pink box), CpGs from prior methylation studies overlapped with one additional annotation (light blue box), and CpGs from GWAS loci overlapped with at least four additional annotations. This resulted in 92,024 “high-value” CpGs. In Step 3, the CpGs were submitted to Illumina for design and manufacture of the final set of selected CpGs. CpGs that passed manufacturing and quality control are shown in Table A. Table A contains 45,891 CpGs, targeted by 53,840 probes.

FIGS. 6A-6B show distribution of Custom Array CpGs by primary criteria and DMR category. (FIG. 6A) CpGs on the Custom array proportionally represented DMR-CpGs from WGBS studies in African Americans, European Americans, and the combined samples. (FIG. 6B) CpGs on the Custom array proportionally represented CpGs from the WGBS studies and previous GWAS of asthma or allergic diseases; very few previous EWAS CpGs were included on the Custom array because nearly all were on the EPIC array.

FIGS. 7A-7C show a comparison of methylation level (β value) distributions from the EPIC and Custom Arrays to the WGBS data. Percent methylation is shown on the x-axis and density is shown on the y-axis. The number of CpGs in each comparison is shown at the top of each pair, which includes the number of sites with at least 10 mapped reads in the WGBS data. (FIG. 7A) β value distribution for the WGBS data compared to the EPIC array for the 19 URECA subjects assayed using both platforms that passed QC. Spearman's rho=0.82 (P<2.2×10⁻¹⁶) (FIG. 7B) β value distribution for the WGBS data compared to the Custom array for the same 19 URECA subjects. Spearman's rho=0.83 (P<2.2×10⁻¹⁶). (FIG. 7C) β value distribution for the WGBS data compared to the Custom array for the 17 COAST samples that passed QC. Spearman's rho=0.82 (P<2.2×10⁻¹⁶)

FIG. 8 shows β value distribution plots from nine GTEX tissues. Percent methylation (β value) is shown on the x-axis and density is shown on the y-axis. Data are from Oliva et al., Genetic regulation of DNA methylation across tissues reveals thousands of molecular links to complex traits (Oliva M et al., Nat Genet. 2023; 55(1):112-22.)

FIGS. 9A-9B show β value distributions of the high-value EPIC CpGs compared to all CpGs on the EPIC array. To determine whether the enrichment for IM CpGs on the Custom array was attributable to the selection criteria we used for designing the array, the CpGs on the EPIC array were filtered using the same pipeline as that used for selecting CpGs for the Custom array. These are CpGs that met criteria for inclusion on the Custom array but were excluded because they were on the EPIC array. These are referred to as high-value EPIC CpGs. Of the 789,290 CpGs on the EPIC array that passed QC, 26,905 (3.4%) were designated as high-value. The β distribution of the high-value EPIC CpGs in nasal epithelial cells revealed a pattern similar to CpGs on the Custom array, with the majority (61%) having β values between 20-80% (FIG. 9A) The β value distribution of the 789,290 EPIC CpGs is shown in blue. (FIG. 9B) The β value distribution of the 26,905 EPIC CpGs that meet the selection criteria for inclusion on the Custom array (high-value EPIC CpGs) is shown in purple. The percent methylation (β value) is shown on the x-axis and density is shown on the y-axis

FIGS. 10A-10B show allergic sensitization in URECA and INSPIRE. (FIG. 10A) Proportion of subjects with positive skin prick test (SPT) results to zero, one to two, three to five, or six to nine allergens tested in the URECA and INSPIRE cohorts. (FIG. 10B) Proportion of positive skin prick tests by allergen tested in URECA and INSPIRE cohorts. The 14 allergens tested for URECA (red) include Mouse epithelia, Dog epithelia, Dermatophagoides fainae (mite), Dermatophagoides pteronyssinus (mite), Cat hair, Rat epithelia, American/German cockroach, German cockroach, Alternaria Tennis, Aspergillus mix, Ragweed mix, Tree pollen (oak or birch), Penicillium Notatum Pennicillium Chrysogenum, Timothy grass. The thirteen allergens tested for INSPIRE (turquoise) include dog, cat, Dermatophagoides pteronyssinus and Dermatophagoides farinae mix, American/German cockroach, Penicillium Notatum Penicillium Chrysogenum, Alternaria Tenuis, Cladosporum Herbarum, Aspergillus mix, Ragweed mix, Eastern 6 Tree mix, K-O-T Grass mix, Maple/Box Elder mix, and Weed mix.

FIGS. 11A-11C show Manhattan plots illustrating EWAS results of allergic sensitization Chromosomes 1-22 are shown along the x-axis and −log₁₀ P-values are shown on the y-axis. (FIG. 11A) Results in URECA (EPIC array). Significant DMCs at a q-value threshold of 0.05 are shown in blue. (FIG. 11B) Results in URECA (Custom array). Significant DMCs at a q-value threshold of 0.05 are shown in red. (FIG. 11C) Results in INSPIRE (Custom array). Significant DMCs at a q-value threshold of 0.05 are shown in turquoise. Plots were generated using CMplot (https:/github.com/YinLiLin/CMplot).

FIGS. 12A-12D show density plots showing β value distributions for all CpGs, DMCs, eQTMs and their nearest genes, and eQTMs and their pcHi-C target genes for the EPIC and Custom arrays. All plots show percent methylation (β value) on the x-axis and density on the y-axis. (FIG. 12A) Density plots of all CpGs on the EPIC (blue; N=789,290) and an exemplary Custom (red; N=37,256) arrays. (FIG. 12B) Density plots of all DMCs on the EPIC (N=1,805) and Custom (N=193) arrays. (FIG. 12C) Density plots of eQTMs with their nearest gene on the EPIC (N=87,193) and Custom (N=8,778) arrays. (FIG. 12D) Density plots of eQTMs with their pcHi-C target genes on the EPIC (N=59,542) and Custom arrays (N=9,298) arrays.

FIG. 13 shows an ancestry PCA plot of self-identified race/ethnicity of 280 URECA subjects Ancestry PCA plot of self-identified race/ethnicity of 280 URECA subjects. PC1 and PC2 are shown on the x-axis and y-axis, respectively. The proportion of variance explained is shown in parentheses.

FIGS. 14A-14C show DMCs and eQTMs at β exemplary loci. In each panel, locus zoom plots show the DMCs from the three AS EWAS using the EPIC array (URECA; blue points=non-high value CpGs; purple points=high-value [filtered] CpGs) and Custom array (URECA, red points; INSPIRE, turquoise points). The genomic locations and genes are shown on the x-axis; EWAS p-values are shown on the y-axis. The dashed horizontal lines show the 0.05 q-value threshold for the Custom array in URECA (red) and for the EPIC array in URECA and Custom array in INSPIRE (blue and purple overlaid). The bar code at the top of the plot shows the location of CpGs at this locus on both arrays. The most significant DMC in the region for a given EWAS (cohort and platform; see legend) is illustrated by a diamond; additional DMCs appear as circles. Correlations between methylation levels (x-axis) of the lead Custom and high-value EPIC CpGs in URECA and expression of relevant genes (y-axis) in epithelial cells from URECA children are shown in the upper right of each panel, and boxplots of the proportion of positive skin prick tests (x-axis) and methylation levels for the lead Custom and high-value EPIC CpGs in URECA (from the EWAS) are shown in the lower right of each panel. (FIG. 14AA) CISH locus on chromosome 3. (FIG. 14B) SLC22A5-IRF1 locus on chromosome 5. (FIG. 14C) HDAC7-VDR locus on chromosome 12.f

FIG. 15 shows regional association plots for the 10 most significant DMCs in the URECA EWAS using the Custom array. The genomic locations and genes are shown on the x-axis; EWAS P-values are shown on the y-axis. The dashed horizontal lines show the 0.05 q-value threshold for the Custom array (red) and for the EPIC array (blue). The density of CpGs in the region is shown along the top of each plot. The most significant DMC in a region for a given EWAS (cohort and platform; see legend upper right) is illustrated by a diamond; additional DMCs appear as circles.

FIG. 16 is a graph showing an evaluation of array performance in identifying men exposed to cow barns (an allergen known to cause asthma). The results demonstrate that the array identified 79 differentially methylated cytosines (DMCs) in men exposed to cow barns compared to men not exposed to cow barns.

FIG. 17 is a graph showing an evaluation of array performance in samples obtained from subjects receiving food allergy treatment with Xolair (omalizumab) vs. subjects receiving a placebo treatment for food allergy. The array identified 140 CpGs associated with a differential response to Xolair at 36 weeks following treatment.

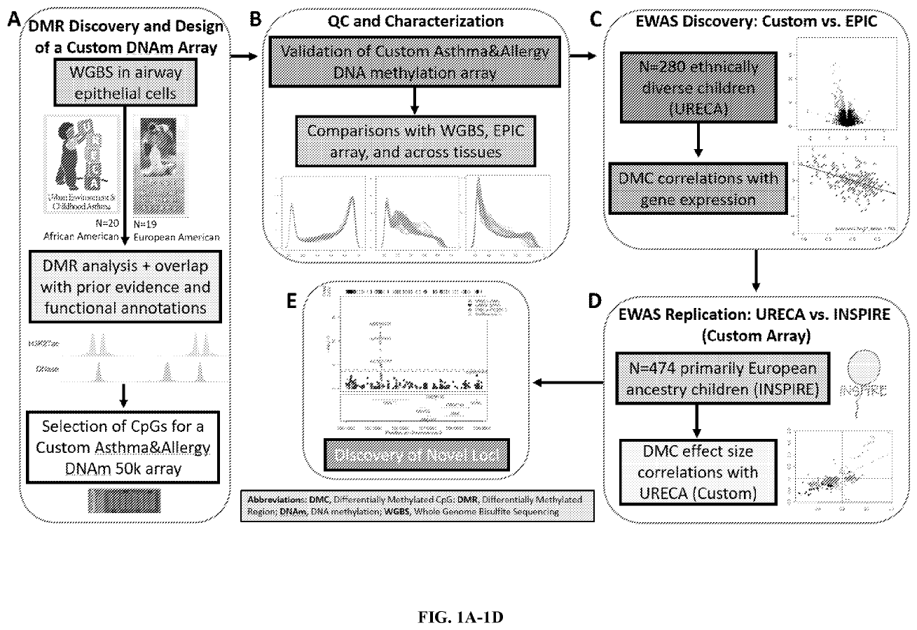

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an oligonucleotide” is a reference to one or more oligonucleotides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “microarray” or “array” refer to a solid support (e.g., a chip, plate, bead, etc.) displaying an arrangement of biomacromolecules. For example, a DNA array displays an arrangement of a plurality of oligonucleotides.

As used herein, the term “genome” refers to all the genetic material in the chromosomes of an organism. DNA derived from the genetic material in the chromosomes of a particular organism is genomic DNA.

As used herein, the term “nucleic acids” refers to any polymer or oligomer of pyrimidine and purine bases, preferably cytosine, thymine and/or uracil, adenine, and guanine, respectively. See Albert L. Lehninger, PRINCIPLES OF BIOCHEMISTRY, at 793-800 (Worth Pub. 1982); incorporated by reference in its entirety. Embodiments herein may utilize any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA. In particular embodiments, the arrays herein and the nucleic acids to be analyzed are deoxyribonucleotides (DNA).

As used herein, the term “oligonucleotide” refers to a nucleic acid (e.g., a DNA polymer) ranging from at least 2 nucleotides in length up to about 50 nucleotides in length (e.g., 2, 3, 4, 5, 6, 7, 8, 9, 10, 11, 12, 13, 14, 15, 16, 17, 18, 19, 20, 25, 30, 35, 40, 45, 50, or ranges therebetween).

As used herein, the term “polynucleotide” refers to a nucleic acid (e.g., a DNA polymer) of 50 nucleotides or more in length.

As used herein, the term “complementary” refers to the capacity for Watson-Crick base pairing between nucleotides or nucleic acids, such as, for instance, between the two strands of a double stranded DNA molecule or between an oligonucleotide probe and a target or sample nucleic acid. “Watson-Crick” base pairing refers to, A and T (or A and U), or C and G. Other forms of base pairing, such as Hoogstein base pairing are not considered complementary herein. Two single nucleic acid molecules are said to be complementary when the nucleotides of one strand, optimally aligned and compared and with a segment of a second strand, allow for 100% Watson-Crick base pairing over the length of the shorter strand. Nucleic acids with less than 100% complementarity (e.g., 95%, 90%, 85%, 80%, 75%, 70%, or less) may still be capable of hybridizing with each other. In some embodiments, a portion of one nucleic acid is complementary to a portion of a second nucleic acid, but the full-lengths of the nucleic acids are not complementary. “Selective hybridization” will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

As used herein, the term “hybridization” refers to the process in which two single-stranded nucleic acids bind non-covalently to form a double-stranded nucleic acid; triple-stranded hybridization is also theoretically possible. The resulting (usually) double-stranded polynucleotide is a “hybrid.” Hybridizations may performed under “stringent conditions,” for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25° C.-30° C. are suitable for allele-specific probe hybridizations or conditions of 100 mM MES, 1 M Na*, 20 mM EDTA, 0.01% Tween-20 and a temperature of 30° C.-50° C., preferably at about 45° C.-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. General hybridization conditions suitable for DNA microarrays are understood in the field

As used herein, the term “hybridization probes” refers to oligonucleotides capable of binding in a base-specific manner to a complementary strand of nucleic acid.

As used herein, the term “label” refers to a molecular entity capable of being attached (covalently or non-covalently) to a target molecule (e.g., a nucleic acid) and being detected (e.g., fluorescence, luminescence, radioactivity, etc.) or bound by a secondary agent (e.g., a hapten capable of being bound by an antibody). Fluorescent labels that find use in embodiments herein include, inter alia, xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorescent label is a small molecule fluorescent label.

As used herein, the term “solid support”, “support”, and “substrate” are used interchangeably and refer to a material or group of materials having a rigid or semi-rigid surface or surfaces. In some embodiments, at least one surface of the solid support will be flat, although in some embodiments it may be desirable to physically separate regions of a surface with, for example, wells, raised regions, etched trenches, or the like. According to certain embodiments, the solid support(s) will take the form of beads, plates, chips, resins, gels, microspheres, or other geometric configurations.

As used herein, the term “target” refers to a molecule that has an affinity for a given probe. In embodiments herein, a target is a nucleic acid and capable of being bound (or suspected of potentially having such capacity) by an oligonucleotide probe herein.

As used herein, the term “endonuclease” refers to an enzyme that cleaves a nucleic acid (DNA or RNA) at internal sites in a nucleotide base sequence. Cleavage may be at a specific recognition sequence, at sites of modification, or randomly.

### DETAILED DESCRIPTION

Provided herein are DNA methylation arrays displaying oligonucleotides containing human CpG sites that are differentially methylated in subjects suffering from asthma and/or allergic disease relative to the general population, and methods of use thereof.

Experiments were conducted during development of embodiments herein to identify methylation sites (e.g., CpGs) in airway epithelial cells that are likely to be functional and associated with asthma and/or allergies in ethnically diverse populations (See ‘Experimental’). Provided herein are arrays displaying a plurality of allele-specific oligonucleotides corresponding to the methylation sites described herein. Methods are provided for using the array to identify and determine the methylation status the methylation sites in a nucleic acid sample.

Table A, filed herewith and incorporated in its entirety provides the genomic coordinates of 45,891 differentially-methylated CpGs identified in the experiments conducted during development of embodiments herein.

In some embodiments, provided herein are reagents capable of determining the methylation status and/or the amount of methylation at one or more of the positions (sequences) provided in Table A. In some embodiments, such reagents comprise oligonucleotide primers and/or probes. In some embodiments, provided herein are oligonucleotides (e.g., probes or primers) capable of hybridizing to a segment of human genomic DNA comprising a genomic position listed in Table A.

In some embodiments, an oligonucleotide herein is complementary to a sequence identified in Table A. In some embodiments, oligonucleotides are complementary to a human genomic DNA sequence and terminate at a position identified in Table A. In some embodiments, oligonucleotides are provided that are complementary to a human genomic DNA sequence encompassing a genomic coordinate identified in Table A.

In some embodiments, provided herein are reagents capable of determining the methylation status and/or the amount of methylation at one or more of the positions (sequences) provided in Table A. In some embodiments, such reagents comprise oligonucleotide primers and/or probes. In some embodiments, provided herein are oligonucleotides (e.g., probes or primers) capable of hybridizing to a segment of human genomic DNA comprising a genomic position listed in Table A. In some embodiments, an oligonucleotide herein is complementary to a sequence identified in Table A. In some embodiments, oligonucleotides are complementary to a human genomic DNA sequence and terminate at a position identified in Table A. In some embodiments, oligonucleotides are provided that are complementary to a human genomic DNA sequence encompassing a genomic coordinate identified in Table A.

In some embodiments, provided herein are oligonucleotides (e.g. oligonucleotide probes) comprising single-stranded DNA sequences. In some embodiments, provided herein are oligonucleotides comprising a sequence having at least 90% sequence identity to (e.g. at least 90%, at least 91%, at least 92%, at least 93%, at least 94%, at least 95%, at least 96%, at least 97%, at least 98%, at least 99%, or 100% identity to) SEQ ID NO: 1-SEQ ID NO: 53,840. In some embodiments, provided herein are oligonucleotides comprising a sequence selected from SEQ ID NO: 1-SEQ ID NO: 53,840.

In some embodiments, provided herein are nucleic acid sequences and allele specific oligonucleotides comprising or complementary to such sequences. In some embodiments, the sequence and allele specific oligonucleotides find use in probing the methylation status of nucleic acids in a sample (e.g., human genomic DNA).

In some embodiments, provided herein are arrays displaying a plurality (e.g., 100, 500, 1000, 5000, 10000, 15,000, 20,000, 25,000, 30,000, 35,000, 40,000, 45,000, 50,000, 55,000, 60,000, 65,000, 70,000, 75,000, 80,000, 85,000, 90,000, or more, or ranges therebetween) of separate oligonucleotides representing methylation sites in the genome of a species (e.g., the human genome). In some embodiments, the oligonucleotides displayed on the array are 15-75 nucleotides in length (e.g., 15. 20. 25, 30, 35, 40, 45, 50, 55, 60, 65, 70, 75, or ranges therebetween). In some embodiments, the oligonucleotides are DNA oligonucleotides. In some embodiments, the 5′ end of the oligonucleotide is linked to the solid surface of the array (directly or indirectly) and the 3′ end of the oligonucleotide is free. In some embodiments, the oligonucleotides are allele-specific oligonucleotides. In some embodiments, an oligonucleotide on the array corresponds to the sequence adjacent to a methylation site, terminating at a position corresponding to the first or second position of the CpG (e.g., the C or G position). In some embodiments, the 3′ end of an oligonucleotide probe displays a sequence (e.g., terminal nucleotide or dinucleotide) capable of Watson/Crick base pairing with the query sequence that results from the methylated and/or unmethylated CpG (e.g., CG or CA).

In some embodiments, two oligonucleotide types are provided on the array for each methylation site, one “methylated” and one “unmethylated” query probe. In some embodiments, the 3′ end of a first type of oligonucleotide probe displays a sequence (e.g., terminal nucleotide) capable of pairing with the query sequence and terminating in a CA dinucleotide (a sequence capable of Watson/Crick base pairing with the TG dinucleotide that results from bisulfite conversion and amplification of an unmethylated CpG). In some embodiments, the 3′ end of a second type oligonucleotide probe displays a sequence (e.g., terminal nucleotide) capable of pairing with the query sequence and terminating in a CG dinucleotide (a sequence capable of Watson/Crick base pairing with the CG dinucleotide that results from protection from bisulfite conversion by a methylated CpG).

In some embodiments, a single oligonucleotide type is provided on the array for each methylation site. In some embodiments, the 3′ end of the oligonucleotide probe displays a sequence that terminates in a C nucleotide corresponding the position complementary to the G of the CpG.

Table A contains the genomic locations of the differentially methylated CpGs identified in the experiments conducted during development of embodiments herein. In some embodiments, provided herein are oligonucleotide probes that hybridize to methylated and/or unmethylated versions of the CpG sites of Table A. In some embodiments, oligonucleotide probes are provided that are complementary to the sequence adjacent to the CpG sites of Table A, terminating at a position corresponding to the first or second position of the CpG (e.g., the C or G position). In some embodiments, the 3′ end of an oligonucleotide probe displays a sequence (e.g., terminal nucleotide or dinucleotide) capable of Watson/Crick base pairing with the query sequence that results from a methylated and/or unmethylated CpG (e.g., CG or CA) of Table A.

In some embodiments, provided herein is a system or composition (e.g., array) comprising 100 or more (e.g., 100, 200, 500, 1,000, 2,000, 5,000, 10,000, 20,000 30,000 40,000, 50,000, 60,000, 70,000, 80,000, 90,000, or more, or ranges therebetween) oligonucleotide probes capable of hybridizing to human genomic DNA (e.g., differentially modified DNA) and revealing the methylation status of a sequence identified in Table A. In some embodiments, the array comprises more than 10,000 oligonucleotide probes. In some embodiments, the array comprises more than 20,000 oligonucleotide probes. In some embodiments, the array comprises more than 30,000 oligonucleotide probes. In some embodiments, the array comprises more than 40,000 oligonucleotide probes. In some embodiments, the array comprises about 50,000 oligonucleotide probes. In some embodiments, the probes are at least 70% complementary (e.g., >70%, >75%, >80%, >85%, >90%, >95%, 100%) to a human genomic sequence at a position identified in Table A (e.g., overlapping the sequence, tenrinating at the sequence, etc.). In some embodiments, the probes are at least 70% identical (e.g., >70%, >75%, >80%, >85%, >90%, >95%, 100%) to a human genomic sequence at a position identified in Table A (e.g., overlapping the sequence, terminating at the sequence, etc.). In some embodiments, a probe herein comprises a sequence that is capable of hybridizing (e.g., under stringent conditions) to a sequence of 5-75 nucleotides in length (e.g., 5, 10, 15, 20, 25, 30, 35, 40, 45, 50, 55, 60, 65, 70, 75, or ranges therebetween) corresponding to a genomic coordinate provided in Table A. In some embodiments, provided herein is a system (e.g. an array) or a composition comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides comprising a distinct sequence having at least 90% sequence identity to (e.g. at least 90%, at least 91%, at least 92%, at least 93%, at least 94%, at least 95%, at least 96%, at least 97%, at least 98%, at least 99%, or 100% identity to) SEQ ID NO: 1-SEQ ID NO: 53,840. In some embodiments, provided herein is a system (e.g. an array) or a composition comprising 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides comprising a distinct sequence selected from SEQ ID NO: 1-SEQ ID NO: 53,840.

In some embodiments, the system (e.g. array) comprises type I and/or type II oligonucleotides (including type II oligonucleotide pairs). In some embodiments, the system (e.g. array) comprises 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) type I oligonucleotides. SEQ ID NO: 1-SEQ ID NO: 37942 correspond to type I probe oligonucleotides. In some embodiments, the system (e.g. array) comprises 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) type II probe oligonucleotides. SEQ ID NO: 37,943-SEQ ID NO: 53,840 correspond to type II probe oligonucleotides. In some embodiments, the system (e.g. array) comprises type I and type II probe oligonucleotides. In some embodiments, the system (e.g. array) comprises type II probe oligonucleotide pairs. In some embodiments, the system (e.g. array) comprises type I probe oligonucleotides and type II probe oligonucleotide pairs. A type II probe oligonucleotide pair refers to the two probe oligonucleotides used to detect a given target. Type II probe oligonucleotide pairs are exemplified by two sequential sequences within SEQ ID NO: 37,943-SEQ ID NO: 53,840, starting with a first pair shown in SEQ ID NO: 37,493 and SEQ ID NO: 37,494, a second pair shown in SEQ ID NO: 37,495 and SEQ ID NO: 37,496, a third pair shown in SEQ ID NO: 37,497 and SEQ ID NO: 37,498, and so on.

In some embodiments, a probe herein comprises a segment capable of hybridizing to a genomic location identified in Table A and one or more other segments. In some embodiments, the one or more other segments are a linker (e.g., for tethering the probe to a solid surface (e.g., plate, bead, etc.), a barcode, a primer-binding sequence, a capture sequence (e.g., capable of hybridizing to a capture oligo), etc. In some embodiments, a probe herein comprises a sequence having at least 90% sequence identity to (e.g. at least 90%, at least 91%, at least 92%, at least 93%, at least 94%, at least 95%, at least 96%, at least 97%, at least 98%, at least 99%, or 100% identity to) SEQ ID NO: 1-SEQ ID NO: 53,840. In some embodiments, a probe herein comprises a sequence selected from SEQ ID NO: 1-SEQ ID NO: 53,840.

In some embodiments, the systems and methods herein comprise arrays, e.g., for the detection and analysis of nucleic acids. In some embodiments, the arrays are high density arrays that can allow simultaneous analysis (e.g., parallel rather than serial processing) of a plurality of samples. In some embodiments, a device comprises a composite array comprising a plurality of individual arrays, and configured to allow processing of multiple samples, as is generally outlined in U.S. Ser. No. 09/256,943, incorporated by reference in its entirety. For example, in some embodiments, individual arrays are present within each well of a microtiter plate. Thus, depending on the size of the microtiter plate and the size of the individual array, very high numbers of assays can be run simultaneously.

Composite arrays can be configured in numerous ways that are understood in the field. In some embodiments, a first substrate comprising a plurality of assay locations (sometimes also referred to herein as “assay wells”), such as a microtiter plate, is configured such that each assay location (microtiter well) contains an individual array. For example, the plastic material of the microtiter plate can be formed to contain a plurality of “bead wells” in the bottom of each of the assay wells. Beads containing the oligonucleotide probes of the invention are loaded into the bead wells in each assay location. Alternatively, oligonucleotide probes are bound directly or via hybridization with a capture probe to discrete spots in the assay well.

In a “two component” array system, the individual arrays are formed on a second substrate, which then can be fitted into the first microtiter plate substrate. For example, fiber optic bundles form the individual arrays, generally with “bead wells” etched into one surface of each individual fiber, such that the beads containing the capture probes are loaded onto the end of the fiber optic bundle. The composite array thus comprises a number of individual arrays that are configured to fit within the wells of a microtiter plate.

Certain embodiments herein utilize a bead-based analytic chemistry system in which beads, also termed microspheres, carrying different chemical functionalities are distributed on a substrate comprising a patterned surface of discrete sites that can bind the individual microspheres. The beads are generally put onto the substrate randomly, and thus several different methodologies can be used to “decode” the arrays. In one embodiment, unique optical signatures are incorporated into the beads, generally fluorescent dyes, that are used to identify the chemical functionality on any particular bead. This allows the synthesis of the nucleic acids to be divorced from their placement on an array, i.e. the oligonucleotide probes are synthesized on the beads, and then the beads are randomly distributed on a patterned surface. Since the beads are first coded with an optical signature, this means that the array can later be “decoded”, i.e. after the array is made, a correlation of the location of an individual site on the array with the probe at that particular site can be made. In such embodiments, the beads may be randomly distributed on the array, a fast and inexpensive process as compared to in situ synthesis or spotting techniques of the prior art. These methods are generally outlined in PCTs U.S. Ser. No. 98/05025 and U.S. Ser. No. 99/14387 and U.S. Ser. Nos. 08/818,199 and 09/151,877, all of which are expressly incorporated herein by reference. Other systems and methods described in U.S. Pat. No. 6,355,431 (incorporated by reference in its entirety) also find use in embodiments herein.

In some embodiments, the DNA arrays described herein are provided as bead arrays. Composition, devices, and methods, including bead arrays and the use thereof, are described, for example in U.S. Pat. Nos. 6,355,431 and 6,429,027; each of which is incorporated herein by reference in their entireties. In bead arrays, microwells (beadwells) are formed in the surface of the array substrate (e.g., by etching). In some embodiments, thousands (e.g., 1,000, 2,000, 5,000, 10,000, 20,000, 50,000, or ranges therebetween), hundreds of thousands (e.g., 100,000, 200,000, 500,000 or ranges therebetween), or millions of wells are formed in the surface. In some embodiments, each well is sized to accommodate and house a single bead or particle. Ise a bead is 1-10 microns in diameter (e.g., 1 μm, 2 μm, β μm, 4 μm, 5 μm, 6 μm, 7 μm, 8 μm, 9 μm, 10 μm, or ranges therebetween). In some embodiments, a bead displays one or more (e.g., 1, 2, 5, 10, 20, 50, 100, 200, 500, or more) copies of a probe oligonucleotide described herein. In some embodiments, a bead displays one or more (e.g., 1, 2, 5, 10, 20, 50, 100, 200, 500, or more) distinct probe oligonucleotides described herein. In some embodiments, a bead displays a probe oligonucleotide capable a hybridizing to a particular methylation site identified in the experiments conducted during development of embodiments herein, and capable of single base extension of the amplicon of the differentially modified version of the methylated or unmethylated variant thereof. In some embodiments, a bead displays two probe oligonucleotides capable a hybridizing to a particular methylation site identified herein, and one probe capable of single base extension of the amplicon of the differentially modified version of the methylated variant thereof and the second probe capable of single base extension of the amplicon of the differentially modified version of the unmethylated variant thereof. In some embodiments, a bead displays a probe oligonucleotide capable a hybridizing to a particular methylation site identified in the experiments conducted during development of embodiments herein, and capable of single base extension of the amplicon of the differentially modified version of the methylated and unmethylated variant thereof.

In some embodiments, methods are provided for determining the methylation status of CpG sites with a sample nucleic acid. In some embodiments, using probes designed for the asthma- and allergy-related methylation sites identified in experiments conducted during development of embodiments herein, the methylation status of human genomic DNA is analyzed. In some embodiments, the sample nucleic acid is treated (e.g., chemically or enzymatically) to differentiate methylated vs. unmethylated sites, the treated nucleic acid is then applied to the array (e.g., following amplification and/or fragmentation), and hybridization of the sample nucleic acid to the oligonucleotide probes on the array is analyzed. Analysis of the locations bound on the array and/or whether the hybridized nucleic acid will support single nucleotide extension (and which nucleotide is added) reveals the methylation status of the nucleic acid in the sample. In some embodiments, the methylation status of multiple (e.g., 1,000, 2,000, 5,000, 10,000, 20,000, 50,000, or more) asthma- and allergy-related methylation sites in the sample DNA is determined suing the methods herein.

In some embodiments, target nucleic acids are modified in a methylation-selective manner (e.g., methylated sites are modified and non-methylated sites are not, or non-methylated sites are modified and methylated sites are not). In particular embodiments, methylated cytosines are distinguished from non-methylated cytosines based on their differential reactivity with bisulfite in which case the latter are converted to uracil and the former are protected from conversion. Nucleic acids in a sample that has been treated with bisulfite are detected using arrays as exemplified herein for detecting single nucleotide polymorphisms or the nucleic acids can be sequenced on arrays. Array detection is used to distinguish whether a uracil is present at a site, which is indicative of unmethylated cytosine in the original sample, or whether a cytosine is present at such a site, which is indicative of a methylated cytosine in the original sample. In alternative embodiments, methylation is detected using the arrays described herein to distinguish different fragments resulting from treating a nucleic acid sample with methylation-specific enzymes, such as methylation sensitive restriction endonucleases.

The arrays herein are utilized in any suitable methods for analyzing the methylation of a target nucleic acid.

In some embodiments, methylation status of a target nucleic acid is conducted by treating the target nucleic acid with a methylation-specific enzyme that discriminates between methylated and unmethylated sites by cleaving at one but on the other. For example, methylation sensitive restriction endonucleases are not able to cleave methylated-cytosine residues, leaving methylated DNA intact.

In some embodiments, a method of methylation detection assays includes digesting genomic DNA with a methylation-sensitive restriction enzyme followed by detection of the differentially cleaved DNA. For example, the methylation specific enzyme HpaII recognizes and cleaves at the sequence 5′-CCGG-3′. However, the digestion is blocked by methylation at either C.

In some embodiments, discrimination of methylated vs unmethylated sites is based on the selective deamination of cytosine to uracil by treatment with bisulfite. The method utilizes bisulfite-induced modification of genomic DNA, under conditions whereby cytosine is converted to uracil, but 5-methylcytosine remains non-reactive. The sequence under investigation is then analyzed by the methods described herein.

In some embodiments, approximately 0.1-100 μg (e.g., 0.1, 0.2, 0.5, 1, 2, 5, 10, 20, 50, 100, or ranges therebetween) of genomic DNA is used in bisulfite conversion to convert the unmethylated cytosine into uracil. The product contains unconverted cytosine where they were previously methylated, but cytosine converted to uracil if they were previously unmethylated.

In some embodiments, the methods disclosed herein utilize nucleic acid amplification in one or more steps. Nucleic acid samples may be derived, for example, from total nucleic acid from a cell or sample, total RNA, cDNA, genomic DNA or mRNA. Many methods of analysis of nucleic acid employ methods of amplification of the nucleic acid sample prior to analysis, and various techniques for nucleic acid amplification are understood in the field. A number of methods for the amplification of nucleic acids have been described, for example, exponential amplification, linked linear amplification, ligation-based amplification, and transcription-based amplification. An example of exponential nucleic acid amplification method is polymerase chain reaction (PCR) which has been disclosed in numerous publications and it utilized in some embodiments herein. See, for example, Mullis et al. Cold Spring Harbor Symp. Quant. Biol. 51:263-273 (1986); and U.S. Pat. Nos. 4,582,788 and 4,683,194; incorporated by reference in its entirety.

Nucleic acid amplification may be carried out through multiple cycles of incubations at various temperatures, i.e., thermal cycling or PCR, or at a constant temperature (an isothermal process). An example of an isothermal amplification technique involves a single, elevated temperature using a DNA polymerase that contains the 5′ to 3′ polymerase activity but lacks the 5′ to 3′ exonuclease activity. As the new strand of DNA is synthesized from the template strand of DNA, the complementary strand of the DNA target is displaced from the original DNA helix. The use of specific primers that invade the target DNA strand allows for self-sustaining amplification and detection techniques and can detect very low copy targets. Isothermal amplification methods, such as strand displacement amplification (SDA), are disclosed in U.S. Pat. Nos. 5,648,211, 5,824,517, 6,858,413, 6,692,918, 6,686,156, 6,251,639 and 5,744,311 and U.S. Patent Pub. No. 20040115644 and in Walker et al. Proc. Natl. Acad. Sci. U.S.A. 89: 392-396 (1992); Guatelli, J. C. et al. Proc. Natl. Acad. Sci. USA 87:1874-1878 (1990), which are incorporated herein by reference in their entirety.

When a pair of amplification primers is used, each of which hybridizes to one of the two strands of a double stranded target sequence, amplification is exponential. This is because the newly synthesized strands serve as templates for the opposite primer in subsequent rounds of amplification. When a single amplification primer is used, amplification is linear because only one strand serves as a template for primer extension and newly synthesized strands are not used as template. Amplification methods that proceed linearly during the course of the amplification reaction are less likely to introduce bias in the relative levels of different mRNAs than those that proceed exponentially. “Single-primer amplification” protocols have been reported in many patents (see, for example, U.S. Pat. Nos. 5,554,516, 5,716,785, 6,132,997, 6,251,639, and 6,692,918 which are incorporated herein by reference in their entirety).

Nucleic acid amplification techniques may be grouped according to the temperature requirements of the procedure. Certain nucleic acid amplification methods, such as the polymerase chain reaction (PCR, Saiki et al., Science, 230:1350-1354, 1985), ligase chain reaction (LCR, Wu et al., Genomics, 4:560-569, 1989; Barringer et al., Gene, 89:117-122, 1990; Barany, Proc. Natl. Sci. USA, 88:189-193, 1991), transcription-based amplification (Kwoh et al., Proc. Natl. Acad. Sci., USA, 86:1173-1177, 1989) and restriction amplification (U.S. Pat. No. 5,102,784), require temperature cycling of the reaction between high denaturing temperatures and somewhat lower polymerization temperatures. In contrast, methods such as self-sustained sequence replication (Guatelli et al., Proc. Natl. Acad. Sci. USA, 87:1874-1878, 1990), the Qβ replicase system (Lizardi et al., BioTechnology, 6:1197-1202, 1988), and Strand Displacement Amplification (SDA—Walker et al., Proc. Natl. Acad. Sci. USA, 89:392-396, 1992a, Walker et al., Nuc. Acids. Res., 20:1691-1696, 1992b; U.S. Pat. No. 5,455,166) are isothenrial reactions that are conducted at a constant temperature, which are typically much lower than the reaction temperatures of temperature cycling amplification methods.

In some embodiments, methods herein utilize PCR techniques understood in the field to amplify genomic DNA. In some embodiments, genomic DNA or other target nucleic acids are amplified by a PCR technique following methylation-status dependent modification of the DNA. For example, a nucleic acid (e.g., genomic DNA) is treated with bisulfite under conditions that modify unmethylated cytosine into uracil, but do not modify methylated cytosine. Amplification of the bisulfite-treated nucleic acid by PCR results in replacement of the uracil nucleotides with thymine nucleotides in the resulting amplicons. Unmethylated CpG sites in the sample nucleic acid are TpG sites in the amplicon; methylated CpG sites in the sample nucleic acid are unmethylated CpG sites in the amplicon.

The bisulfite treated DNA is subjected to whole-genome multiple displacement amplification via random hexamer priming and Phi29 DNA polymerase, which has a proofreading activity resulting in error rates 100 times lower than the Taq polymerase. The products are then enzymatically fragmented, purified from dNTPs, primers and enzymes, and applied to the chip.

In some embodiments, target nucleic acids, differentially-modified nucleic acids, and/or amplicons thereof are fragmented to produce target oligonucleotides or polynucleotides for subsequent analysis. A fragmentation process produces DNA fragments within a certain range of length (e.g., that can subsequently be labeled and analyzed). In some embodiments, the average size of fragments obtained from fragmentation are at least 10, 20, 30, 40, 50, 60, 70, 80, 100, 200 nucleotides, or ranges therebetween. Fragmentation of nucleic acids comprises breaking nucleic acid molecules into smaller fragments. Fragmentation of nucleic acid may be desirable to optimize the size of nucleic acid molecules for certain reactions and analyses and reduce three dimensional structures. For example, fragmented nucleic acids may be used for more efficient hybridization of oligonucleotide probes. According to some embodiments herein, before hybridization to a microarray, target nucleic acid (e.g., amplicons of differentially-modified sample DNA) is fragmented to sizes ranging from about 50 to 200 bases long, for example, to improve target specificity and sensitivity.

In some embodiments, differentially-modified nucleic acids, amplicons thereof, and/or fragments thereof are analyzed by hybridization to oligonucleotide probes (e.g., presented on an array). In some embodiments, multiple copies of target DNA generated by the methods herein and understood in the field are analyzed by hybridization to an array of oligonucleotide probes.

In some embodiments, there are two bead types present on an array for each CpG site. Each locus tested is differentiated by different bead types. Both bead types are attached to single-stranded DNA oligonucleotides (e.g., 10⁻¹⁰⁰mer (e.g., 10, 20, 30, 40, 50, 60, 70, 80, 90, or 100 nucleotides in length, or ranges therebetween) that differ in sequence only at the free end; this type of probe is known as an allele-specific oligonucleotide. One of the bead types will correspond to the methylated cytosine locus and the other will correspond to the unmethylated cytosine locus, which has been converted into uracil during bisulfite treatment and later amplified as thymine during whole-genome amplification. The bisulfite-converted amplified DNA products are denatured into single strands and hybridized to the chip via allele-specific annealing to either the methylation-specific probe or the non-methylation probe. Hybridization is followed by single-base extension with hapten-labeled dideoxynucleotides (or fluorescently labelled dideoxynucleotides). In some embodiments, the ddCTP and ddGTP are labeled with biotin while ddATP and ddUTP are labeled with 2,4-dinitrophenol (DNP). In other embodiments, ddNTPs are each labeled with different fluorophores. The dideoxynucleotides are capable of terminating DNA synthesis after a single base extension.

In some embodiments, target nucleic acids, differentially-modified nucleic acids, amplicons thereof, and/or fragments thereof that have been specifically hybridized to the arrayed oligonucleotide probes are used as templates in a single-base extension (SBE) reaction. SBE provides a method for determining the identity of a nucleotide base at a specific position along a nucleic acid. The method has commonly been used to identify single-nucleotide polymorphisms (SNPs). In some embodiments, a hybridization complex is formed between the substrate-displayed oligonucleotide probe and a complementary region of the target nucleic acid (e.g., differentially-modified, amplified, and fragmented target DNA), such that the 3′ terminal nucleotide of the probe oligonucleotide is either adjacent to the position in the target nucleic acid to be analyzed (e.g., the position to be analyzed in the first position in the target nucleic acid not hybridized to a base in the probe) or in position to hybridize to (if they are complementary bases) the position in the target nucleic acid to be analyzed. Using a DNA polymerase, the oligonucleotide probe is enzymatically extended by a single base in the presence of all four nucleotide terminators (e.g., labeled (e.g., fluorescently labelled)); the nucleotide terminator complementary to the base in the template being interrogated is incorporated and identified. However, if the 3′ terminal nucleotide of the probe oligonucleotide is not base-paired with the corresponding base in the target (e.g., because they are not complementary bases), then the extension will not occur. Many approaches can be taken for determining the identity of an incorporated terminator, including fluorescence labeling, mass labeling for mass spectrometry, isotope labeling, and tagging the base with a hapten and detecting chromogenically with an anti-hapten antibody-enzyme conjugate

In some embodiments, after incorporation of these hapten-labeled ddNTPs, multi-layered immunohistochemical assays are performed by repeated rounds of staining with a combination of antibodies to differentiate the two types. After staining, the chip is scanned to show the intensities of the unmethylated and methylated bead types. The raw data are analyzed by the proprietary software, and the fluorescence intensity ratios between the two bead types are calculated. For a given individual at a given locus, a ratio value of 0 equals to non-methylation of the locus (i.e., homozygous unmethylated); a ratio of 1 equals to total methylation (i.e., homozygous methylated); and a value of 0.5 means that one copy is methylated and the other is not (i.e., heterozygosity), in the diploid human genome. In embodiments in which fluorescently or otherwise detectably-labeled nucleotides (e.g., ddNTPs) are added in the SBE reaction, the staining step is eliminated.

The scanned microarray images of methylation data are further analyzed by systems and methods herein, which normalizes the raw data to reduce the effects of experimental variation, background and average normalization, and performs standard statistical tests on the results. The data can then be compiled into several types of figures for visualization and analysis. Scatter plots are used to correlate the methylation data; bar plots to visualize relative levels of methylation at each site tested; heat maps to cluster the data to compare the methylation profile at the sites tested.

In some embodiments, provided herein are methods of detecting the presence of nucleic acid sequences in a sample, comprising: (a) contacting probe oligonucleotides described herein (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) probe oligonucleotides, each of the 1,000 or more (e.g., >1,000, >2,000, >5,000, >10,000, >15,000, >20,000, >25,000, >30,000, >35,000, >40,000, >45,000, >50,000, >55,000, >60,000, >65,000, >70,000, >75,000, >80,000, >85,000, >90,000) or a device displaying such probe oligonucleotides with a nucleic acid sample; and (b) detecting the binding of one or more nucleic acids comprising the nucleic acid sequences to one or more of the probe oligonucleotides.

In some embodiments, provided herein are methods of detecting the methylation status of methylation sites in a nucleic acid in a sample, the method comprising: (a) treating the sample to differentially modify the nucleic acid at methylated and unmethylated methylation sites to produce a differentially-modified nucleic acid; (b) amplifying the differentially-modified nucleic acid; (c) fragmenting the differentially-modified nucleic acid into differentially-modified oligonucleotides; (d) contacting a device described herein with the differentially-modified oligonucleotides, and allowing the differentially-modified oligonucleotides to hybridize to the probe oligonucleotides, thereby forming probe/differentially-modified oligonucleotide complexes; (e) labeling the probe/differentially-modified oligonucleotide complexes in a manner that is specific to whether the differentially-modified oligonucleotide of each complex corresponds to a methylated or unmethylated methylation site; (f) detecting the labeled probe/differentially-modified oligonucleotide complexes; and (g) analyzing (1) the type of labeling and (2) the location of the probe/differentially-modified oligonucleotide complexes on the surface.

In some embodiments, amplifying the differentially-modified nucleic acid comprises PCR amplification.

In some embodiments, treating the sample to differentially modify the nucleic acid at methylated and unmethylated methylation sites comprises exposing the sample to bisulfite that converts unmethylated cytosine to uracil but methylated cytosines are protected from conversion. In some embodiments, amplifying the differentially-modified nucleic acid converts the uracil generated by bisulfite conversion into thymine. In some embodiments, treating the sample to differentially modify the nucleic acid at methylated and unmethylated methylation sites comprises exposing the sample to bisulfite that converts unmethylated cytosine to uracil but methylated cytosines are protected from conversion.

In some embodiments, fragmenting the differentially-modified nucleic acid comprises site-specific fragmentation of the differentially-modified nucleic acid. In some embodiments, site-specific fragmentation is by restriction endonuclease. In some embodiments, fragmenting the differentially-modified nucleic acid comprises random fragmentation of the differentially-modified nucleic acid. In some embodiments, random fragmentation comprises chemical, enzymatic, and/or mechanical fragmentation.

In some embodiments, methods further comprise a step of isolating the differentially-modified nucleic acid and/or differentially-modified oligonucleotides from reagents for amplification and/or fragmentation.

In some embodiments, labeling the probe/differentially-modified oligonucleotide complexes in a manner that is specific to whether the differentially-modified oligonucleotide of each complex comprises performing a single nucleotide extension reaction with labeled nucleotides. In some embodiments, the labeled nucleotides comprise happens. In some embodiments, methods further comprise contacting probe/differentially-modified oligonucleotide complexes following the single nucleotide extension with antibodies capable of binding to the haptens, wherein the antibodies comprise detectable labels. In some embodiments, the labeled nucleotides comprise detectable labels. In some embodiments, the detectable labels comprise fluorescent labels.

In some embodiments, nucleic acid samples comprising genomic DNA. In some embodiments, the genomic DNA is human genomic DNA. In some embodiments, the human genomic DNA is obtained from airway epithelial cells. In some embodiments, the cells are obtained from a subject suffering from asthma and/or allergic disease. In some embodiments, the cells are obtained from a subject suspected as having asthma and/or allergic disease. The term “asthma” as used herein refers to a chronic condition involving a narrowing and/or swelling of the bronchial airways, making it difficult for a subject to breathe. The asthma may be intermittent asthma, mild persistent asthma, moderate persistent asthma, or severe persistent asthma. Symptoms of asthma include coughing, wheezing, chest tightness, and/or difficulty breathing. The term “allergies” and “allergic disease” are used interchangeably herein and refer to a hypersensitive immune reaction to an allergen. The allergen may be an environmental allergen (e.g. pollen, mold/spores, dust mites, animals (e.g. mice, dogs, cats, cows, horses, etc.), and the like). The allergen may be a toxin (e.g. bee sting, wasp bite, etc.). The allergen may be a food allergen. The allergen may be a drug (e.g. an antibiotic, a compound, etc.). Common examples of allergic disease include, for instance, food allergy, atopic dermatitis, drug allergies, rhinitis, and hay fever.

In some embodiments, the methods of detecting the presence of nucleic acids in a sample and/or methods of detecting the methylation status of methylation sites in nucleic acids in a sample provided herein (e.g. using an array described herein) are performed to assess whether a subject (e.g. from which the sample is obtained) has or is at risk of having asthma or an allergic disease. In some embodiments, the methods of detecting the presence of nucleic acids in a sample and/or methods of detecting the methylation status of methylation sites in nucleic acids in a sample provided herein (e.g. using an array described herein) are performed to assess whether a subject (e.g. from which the sample is obtained) has been exposed to one or more allergens (e.g. environmental conditions) known to cause asthma. For example, in some embodiments when nucleic acids in a sample obtained from the subject bind to a sufficient number or percentage of the 100 or more probe oligonucleotides (e.g. 1,000 or more, 10,000 or more, 20,000 or more, 30,000 or more, 40,000 or more, 50,000 or more, etc.) on a surface of an array described herein, the subject is determined to have or be at risk of having asthma or allergic disease. As another example, in some embodiments when the sample obtained from the subject contains nucleic acid containing methylation sites that hybridize (after suitable treatment steps, as described herein) to a certain number or percentage of the probes on an array described herein, the subject is indicated as having or at risk of having asthma or allergic disease. In some embodiments, the methods further comprise providing an appropriate treatment to the subject if the subject is determined to have asthma or an allergic disease. In some embodiments, the treatment comprises one or more of corticosteroids, antihistamines, mast cell stabilizers, decongestants, epinephrine, antibodies, immunotherapies, and the like.

In some embodiments, the methods of detecting the presence of nucleic acids in a sample and methods of detecting the methylation status of methylation sites in nucleic acids in a sample provided herein (e.g. using an array described herein) are performed to evaluate or predict a response to a treatment for asthma or allergic disease. For example, in some embodiments the methods are used to assess whether a subject (e.g. from which the sample is obtained) has, is having, or is likely to have a positive response to a treatment for asthma or an allergic disease. For example, in some embodiments the methods find use in methods of determining whether a subject has, is having, or is likely to have a positive response (e.g. is seeing a positive therapeutic effect) for treatments for a food allergy, including antibody-based treatment for food allergy.

### Experimental

### Example 1

### I. Results

### Identifying Differentially Methylated Regions in Whole Genome Bisulfite Sequences

Whole genome bisulfite sequencing (WGBS) was performed in airway epithelial cell DNA from 20 African American children (10 with allergic asthma, 10 without asthma or allergies; 11 years old) from the Urban Environment and Childhood Asthma (URECA) cohort (J. E. Gern et al., The Urban Environment and Childhood Asthma (URECA) birth cohort study: design, methods, and study population. BMC Pulm Med 9, 17 (2009) and 19 European American young adults (9 with allergic asthma, 10 without asthma or allergies; 18-20 years old) from the Childhood Origins of Asthma (COAST) cohort (R. F. Lemanske, Jr., The childhood origins of asthma (COAST) study. Pediatr Allergy Immunol 13, 38-43 (2002). After quality control (QC) (see Methods), analyses for differentially methylated regions (DMRs) between the asthma/allergy cases and non-asthma/non-allergy controls were performed in the African American sample, the European American sample, and the combined sample. CpGs covered by at least 10 reads in 80% or more of individuals in each sample were included, and at least three CpGs and a maximum gap of 300 bp was required to define regional boundaries. DMRs were defined as regions with at least 5% difference in methylation between the asthma/allergy cases and the non-allergy/non-asthma controls. Overall, 16,611 DMRs were identified that included 199,473 CpGs. Additional characteristics of the DMRs in the three analyses are described in Table 1.

TABLE 1
Description of the differentially methylated regions (DMRs)
from the whole genome bisulfite sequencing study.

| | | Median | Median | # CpGs | # CpGs |
|---|---|---|---|---|---|
| Group | | Size (bp) | # CpGs | hyper- | hypo- |
| Analyzed | # DMRs | [range] | [range] | methylated | methylated |
| AA | 7,748 | 483 | 10 | 2,048 | 5,700 |
| | | [6-3,828] | [3-124] | | |
| EA | 8,972 | 437 | 9 | 1,879 | 7,093 |
| | | [8-2,841] | [3-144] | | |
| Combined | 2,585 | 513 | 11 | 498 | 2,087 |
| | | [9-2,929] | [3-163] | | |

AA, African American;
EA, European American

### Selecting High-Value CpGs for an Asthma&Allergy Custom DNA Methylation Array

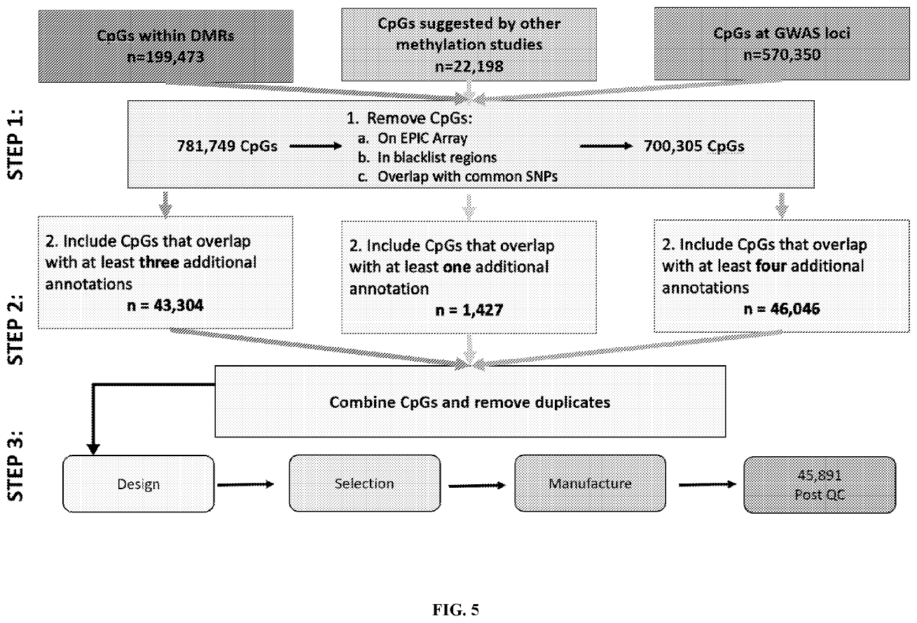

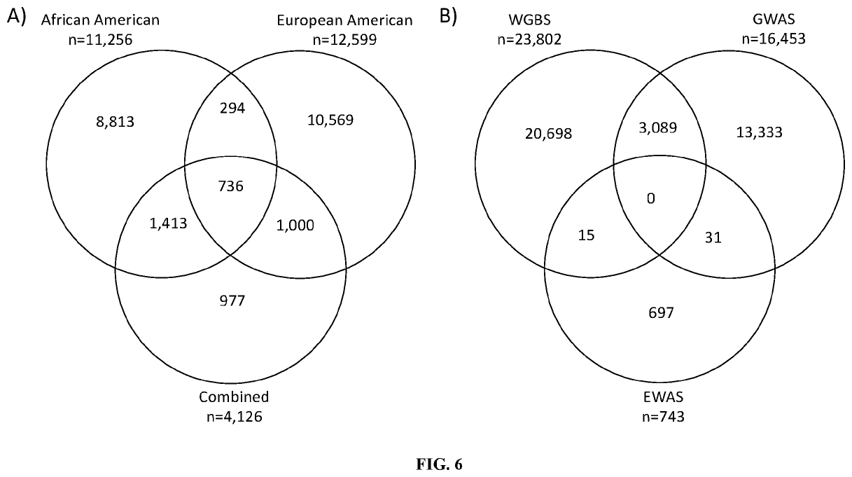

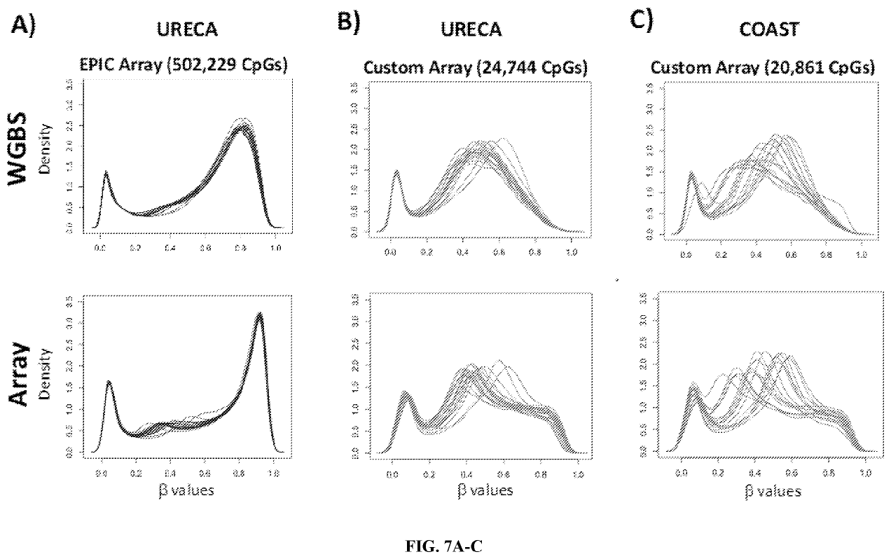

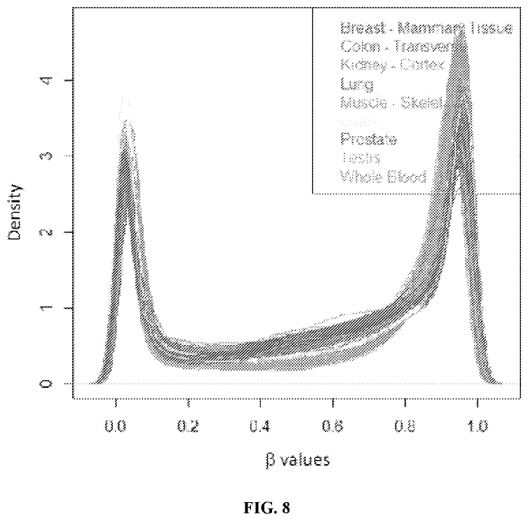

To develop a custom array of CpGs that would complement and serve as a “booster” for the EPIC array (e.g., an example of the arrays within the scope herein), selection criteria involved three steps (FIG. 5). A set of CpGs was identified based on prior evidence of association with asthma or allergic diseases (atopic dermatitis/eczema, allergic rhinitis/hay fever, and food allergy) from three categories of studies. The first category included the 199,473 CpGs within the DMRs from the WGBS study described above, the second category included 19,057 CpGs from 17 previous EWAS (Table 2), and the third category included 570,350 CpGs at 140 GWAS loci (Table 3). CpGs on the EPIC array, in ENCODE blacklisted regions (F. Krueger, S. R. Andrews, Bismark: a flexible aligner and methylation caller for Bisulfite-Seq applications. Bioinformatics 27, 1571-1572 (2011)), and those that overlapped with a common SNP in 1000 Genomes European (CEU) or African (YRI) populations were removed. To further prioritize the remaining 696,225 CpGs, overlap with functional annotations was considered in the second step, requiring that CpGs within DMRs overlap with at least three annotations or other category of prior evidence, CpGs from prior EWAS overlap with at least one annotation or other category of prior evidence, and CpGs at GWAS loci overlap with at least four annotation categories or other category of prior evidence. After removing duplicates, 92,024 high-value CpGs remained for inclusion on a custom array in the third step. After manufacture and array QC, 45,891 CpGs, targeted by 53,840 probes remained (Table A).

TABLE 2
EWAS studies of asthma and allergic diseases used for CpG selection in Step
1. Phenotypes are shown only for those used to select CpGs for the array.

| | | | |
|---|---|---|---|
| Illumina BeadChip | Phenotype(s) | Sample Size | Study |

Respiratory cells

| | | | |
|---|---|---|---|
| EPIC | Allergic rhinitis | 454 | Morin et al. 2020¹ |
| 450K | Remittent asthma vs Persistent asthma or | 135 | Vermeulun et al. |
| | controls | | 2020² |
| EPIC | Asthma, FeNo, total IgE, environmental | 547 | Cardenas et al. 2019³ |
| | IgE, allergic asthma, bronchodilator | | |
| | response | | |
| 450K | Atopy | 483 | Forno et al. 2019⁴ |
| 450K | Atopic asthma | 72 | Yang et al. 2017⁵ |

Blood cells

| | | | |
|---|---|---|---|
| 450K | Inhaled corticosteroids exposure | 215 | Kere et al. 2020⁶ |
| 450K | Allergic sensitization | 376 | Zhang, et al. 2019⁷ |
| 450K | Asthma | 3572 newborns | Reese et al. 2019⁸ |
| | | 2862 children | |
| 450K | Food allergen sensitization, allergen | 739 | Peng et al. 2019⁹ |
| | sensitization, atopic sensitization | | |
| 450K | Childhood asthma | 817 | Xu et al. 2108¹⁰ |
| 450K | Total serum IgE | 217 | Peng et al. 2018¹¹ |
| 450K | Total serum IgE | 306 | Chen et al. 2017¹² |
| 450K | Atopic sensitization and high serum IgE | 367 | Everson et al. 2015¹³ |
| 450k | Eczema | 366 | Quraishi et al. 2015¹⁴ |
| 27K | Serum IgE | 355 | Liang et al. 2015¹⁵ |

¹Morin, A. et al. Epigenetic landscape links upper airway microbiota in infancy with allergic rhinitis at 6 years of age. J Allergy Clin Immunol 146, 1358-1366, doi: 10.1016/j.jaci.2020.07.005 (2020).
²Vermeulen, C. J. et al. Differential DNA methylation in bronchial biopsies between persistent asthma and asthma in remission. Eur Respir J 55, doi: 10.1183/13993003.01280-2019 (2020).
³Cardenas, A. et al. The nasal methylome as a biomarker of asthma and airway inflammation in children. Nat Commun 10, 3095, doi: 10.1038/s41467-019-11058-3 (2019).
⁴Forno, E. et al. DNA methylation in nasal epithelium, atopy, and atopic asthma in children: a genome-wide study. Lancet Respir Med 7, 336-346, doi: 10.1016/S2213-2600(18)30466-1 (2019).
⁵Yang, I. V. et al. The nasal methylome and childhood atopic asthma. J Allergy Clin Immunol 139, 1478-1488, doi: 10.1016/j.jaci.2016.07.036 (2017).
⁶Kere, M. et al. Effects of inhaled corticosteroids on DNA methylation in peripheral blood cells in children with asthma. Allergy 75, 688-691, doi: 10.1111/all.14043 (2020).
⁷Zhang, H. et al. DNA methylation and allergic sensitizations: A genome-scale longitudinal study during adolescence. Allergy 74, 1166-1175, doi: 10.1111/all. 13746 (2019).
⁸Reese, S. E. et al. Epigenome-wide meta-analysis of DNA methylation and childhood asthma. J Allergy Clin Immunol 143, 2062-2074, doi: 10.1016/j.jaci.2018.11.043 (2019).
⁹Peng, C. et al. Epigenome-wide association study reveals methylation pathways associated with childhood allergic sensitization. Epigenetics 14, 445-466, doi: 10.1080/15592294.2019.1590085 (2019).
¹⁰Xu, C. J. et al. DNA methylation in childhood asthma: an epigenome-wide meta-analysis. Lancet Respir Med 6, 379-388, doi: 10.1016/S2213-2600(18)30052-3 (2018).
¹¹Peng, C. et al. Epigenome-wide association study of total serum immunoglobulin E in children: a life course approach. Clin Epigenetics 10, 55, doi: 10.1186/s13148-018-0488-x (2018).
¹²Chen, W. et al. An epigenome-wide association study of total serum IgE in Hispanic children. J Allergy Clin Immunol 140, 571-577, doi: 10.1016/j.jaci.2016.11.030 (2017).
¹³Everson, T. M. et al. DNA methylation loci associated with atopy and high serum IgE: a genome-wide application of recursive Random Forest feature selection. Genome Med 7, 89, doi: 10.1186/s13073-015-0213-8 (2015).
¹⁴Quraishi, B. M. et al. Identifying CpG sites associated with eczema via random forest screening of epigenome-scale DNA methylation. Clin Epigenetics 7, 68, doi: 10.1186/s13148-015-0108-y (2015).
¹⁵Liang, L. et al. An epigenome-wide association study of total serum immunoglobulin E concentration. Nature 520, 670-674, doi: 10.1038/nature14125 (2015).

TABLE 3
Significant loci in GWAS studies of asthma and allergic disease used to define regions for CpG
inclusion. Both studies were performed using data for white British subjects in the UK Biobank.

| Chr | Start¹ | End | Phenotype² | Study³ |
|---|---|---|---|---|
| 1 | 2492665 | 3175371 | Hay Fever | 1 |
| 1 | 8412989 | 9355936 | Hay Fever, Asthma | 1 |
| 1 | 12100942 | 12147311 | Hay Fever | 1 |
| 1 | 12175658 | 12175658 | Asthma | 1 |
| 1 | 25224509 | 25263997 | Hay Fever | 1 |
| 1 | 149897287 | 153166983 | Hay Fever, Asthma, Childhood onset asthma | 1, 2 |
| 1 | 154405024 | 154428283 | Hay Fever | 1 |
| 1 | 161159147 | 161187665 | Hay Fever, Asthma | 1 |
| 1 | 167198536 | 167439010 | Hay Fever, Asthma | 1 |
| 1 | 172777616 | 173171841 | Hay Fever, Asthma, Childhood onset asthma | 1, 2 |
| 1 | 198656242 | 198670555 | Asthma | 1 |
| 1 | 203058476 | 203108508 | Asthma, shared adult and childhood onset asthma | 1, 2 |
| 1 | 212858748 | 212877647 | Hay Fever | 1 |
| 2 | 8438693 | 8496062 | Hay Fever, Asthma, shared adult and childhood onset asthma | 1, 2 |
| 2 | 28623159 | 28644670 | Hay Fever | 1 |
| 2 | 61112552 | 61161095 | Hay Fever | 1 |
| 2 | 102243154 | 103277862 | Hay Fever, Asthma, shared adult and childhood onset asthma | 1, 2 |
| 2 | 112253302 | 112268892 | Hay Fever | 1 |
| 2 | 113582782 | 113689747 | Hay Fever | 1 |
| 2 | 143745800 | 143886819 | Hay Fever | 1 |
| 2 | 146111968 | 146316319 | adult onset asthma | 2 |
| 2 | 198148084 | 198954774 | Hay Fever, Asthma | 1 |
| 2 | 228625484 | 228751874 | Hay Fever, Childhood onset asthma | 1, 2 |
| 2 | 234113057 | 234115739 | Hay Fever | 1 |
| 2 | 242562010 | 242838542 | Hay Fever, Asthma, shared adult and childhood onset asthma | 1, 2 |
| 3 | 32920602 | 33146535 | Hay Fever, Asthma, shared adult and childhood onset asthma | 1, 2 |
| 3 | 50701250 | 51441307 | Asthma | 1 |
| 3 | 72394852 | 72394852 | Hay Fever | 1 |
| 3 | 112526053 | 112693753 | Hay Fever | 1 |
| 3 | 121387784 | 121728846 | Hay Fever | 1 |
| 3 | 127886957 | 128075398 | Asthma | 1 |
| 3 | 141040654 | 141158614 | Hay Fever | 1 |
| 3 | 176708724 | 176868116 | Asthma | 1 |
| 3 | 187632967 | 188457255 | Hay Fever, Asthma, Childhood onsct asthma | 1, 2 |
| 3 | 196327220 | 196454053 | Hay Fever, Asthma | 1 |
| 4 | 4766265 | 4778175 | Hay Fever | 1 |
| 4 | 38599054 | 38934478 | Hay Fever, Asthma, Childhood onset asthma | 1, 2 |
| 4 | 103188709 | 103590864 | Hay Fever | 1 |
| 4 | 122993500 | 124511672 | Hay Fever, Asthma, shared adult and childhood onset asthma | 1, 2 |
| 5 | 14572453 | 14701003 | Hay Fever, Asthma, shared adult and childhood onset asthma | 1, 2 |
| 5 | 35728440 | 36074412 | Hay Fever, Asthma | 1 |
| 5 | 40442869 | 40623346 | Hay Fever | 1 |
| 5 | 71695880 | 71743322 | childhood onset asthma | 2 |
| 5 | 109612633 | 110749926 | Hay Fever, Asthma, shared adult and childhood onset asthma | 1, 2 |
| 5 | 118659579 | 118739934 | Hay Fever, Asthma | 1 |
| 5 | 129917070 | 132138129 | Asthma | 1 |
| 5 | 131336105 | 132321276 | Hay Fever, Childhood onset asthma | 1, 2 |
| 5 | 133439274 | 133639311 | Hay Fever | 1 |
| 5 | 137461112 | 137605401 | Hay Fever | 1 |
| 5 | 141400028 | 141557236 | Hay Fever, Asthma | 1 |
| 5 | 156930406 | 156988798 | Asthma | 1 |
| 5 | 159896259 | 159929015 | Hay Fever, Asthma | 1 |
| 6 | 403799 | 421196 | childhood onset asthma | 2 |
| 6 | 25823774 | 33770370 | Hay Fever, Asthma, shared adult and childhood onset asthma | 1, 2 |
| 6 | 36349890 | 36380644 | Hay Fever | 1 |
| 6 | 90808352 | 91019304 | Hay Fever, Asthma, shared adult and childhood onset asthma | 1, 2 |
| 6 | 128264925 | 128294709 | Hay Fever, Asthma | 1 |
| 6 | 135624811 | 135950204 | Hay Fever, Asthma | 1 |
| 6 | 138002175 | 138262773 | Hay Fever | 1 |
| 6 | 155162163 | 155162163 | Asthma | 1 |
| 7 | 3062629 | 3174209 | Hay Fever, Asthma | 1 |
| 7 | 20371853 | 20640689 | Hay Fever, Asthma, shared adult and childhood onset asthma | 1, 2 |
| 7 | 22755688 | 22811384 | childhood onset asthma | 2 |
| 7 | 28139386 | 28259233 | Hay Fever, Asthma, shared adult and childhood onset asthma | 1, 2 |
| 7 | 76978096 | 77038945 | Hay Fever | 1 |
| 7 | 150690176 | 150690176 | Hay Fever | 1 |
| 8 | 81171813 | 81329123 | Hay Fever, Asthma, shared adult and childhood onset asthma | 1, 2 |
| 8 | 101514998 | 101519901 | Hay Fever | 1 |
| 8 | 128777719 | 128815029 | Hay Fever, Childhood onset asthma | 1, 2 |
| 9 | 5609742 | 6621066 | Hay Fever, Asthma, shared adult and childhood onset asthma | 1, 2 |
| 9 | 16715826 | 16756377 | Hay Fever | 1 |
| 9 | 101790878 | 101820718 | Hay Fever | 1 |
| 9 | 101915887 | 101989706 | Asthma | 1 |
| 9 | 117804027 | 117834931 | Hay Fever | 1 |
| 9 | 123636121 | 123707497 | Hay Fever | 1 |
| 9 | 127022266 | 127095039 | Hay Fever | 1 |
| 9 | 131455796 | 131617167 | Hay Fever, Asthma | 1 |
| 9 | 136141870 | 136155000 | Hay Fever | 1 |
| 9 | 140500443 | 140500443 | childhood onset asthma | 2 |
| 10 | 5885314 | 6631223 | Hay Fever, Asthma, Childhood onset asthma | 1, 2 |
| 10 | 8095340 | 9938970 | Hay Fever, Asthma, shared adult and childhood onset asthma | 1, 2 |
| 10 | 64349979 | 64391375 | Hay Fever | 1 |
| 10 | 94342983 | 94492716 | Asthma | 1 |
| 10 | 104222963 | 104512006 | Hay Fever | 1 |
| 11 | 1110395 | 1147618 | Asthma, shared adult and childhood onset asthma | 1, 2 |
| 11 | 2237219 | 2296012 | Hay Fever | 1 |
| 11 | 36336263 | 36388519 | Hay Fever, Asthma | 1 |
| 11 | 60793330 | 60793722 | Hay Fever | 1 |
| 11 | 61543499 | 61623140 | Asthma | 1 |
| 11 | 61630104 | 61657926 | shared adult and childhood onset asthma | 2 |
| 11 | 65495211 | 65683531 | Hay Fever, Asthma, Childhood onset asthma | 1, 2 |
| 11 | 75891182 | 76377819 | Hay Fever, Asthma, shared adult and childhood onset asthma | 1, 2 |
| 11 | 95419908 | 95426984 | Hay Fever | 1 |
| 11 | 111415822 | 111647084 | Hay Fever, Asthma | 1 |
| 11 | 118550522 | 118770321 | Hay Fever, Childhood onset asthma | 1, 2 |
| 11 | 128131013 | 128200831 | Hay Fever | 1 |
| 12 | 48186563 | 48210318 | Asthma, shared adult and childhood onset asthma | 1, 2 |
| 12 | 55358844 | 57535266 | Hay Fever, Asthma, shared adult and childhood onset asthma | 1, 2 |
| 12 | 71405206 | 71585743 | Asthma, shared adult and childhood onset asthma | 1, 2 |
| 12 | 94556678 | 94604963 | Asthma | 1 |
| 12 | 111708458 | 112906415 | Hay Fever, Childhood onset asthma | 1, 2 |
| 12 | 121133037 | 121410678 | Hay Fever, Childhood onset asthma | 1, 2 |
| 12 | 122645048 | 123829116 | Hay Fever | 1 |
| 13 | 40975005 | 41502588 | Hay Fever | 1 |
| 13 | 44475398 | 44490181 | Asthma | 1 |
| 13 | 50808877 | 50811151 | Hay Fever | 1 |
| 13 | 73359692 | 74039935 | Hay Fever | 1 |
| 13 | 74039935 | 74039935 | Asthma | 1 |
| 13 | 99781378 | 100227069 | Hay Fever, Asthma, shared adult and childhood onset asthma | 1, 2 |
| 14 | 35510900 | 35864878 | Hay Fever | 1 |
| 14 | 68727506 | 68815261 | Hay Fever, Asthma, shared adult and childhood onset asthma | 1, 2 |
| 14 | 103067487 | 103387971 | Hay Fever | 1 |
| 15 | 41252202 | 41796498 | Hay Fever, Asthma | 1 |
| 15 | 61032054 | 61123862 | Hay Fever, Asthma, shared adult and childhood onset asthma | 1, 2 |
| 15 | 67371244 | 67469570 | Hay Fever, Asthma, shared adult and childhood onset asthma | 1, 2 |
| 15 | 75399102 | 75448181 | Hay Fever | 1 |
| 15 | 84556623 | 84556623 | Asthma | 1 |
| 15 | 90859095 | 91094064 | Hay Fever | 1 |
| 16 | 11006011 | 11336508 | Hay Fever, Asthma, shared adult and childhood onset asthma | 1, 2 |
| 16 | 27203012 | 27417744 | Hay Fever, Asthma, shared adult and childhood onset asthma | 1, 2 |
| 16 | 50745926 | 50790158 | Asthma, shared adult and childhood onset asthma | 1, 2 |
| 17 | 4521473 | 4535314 | Hay Fever | 1 |
| 17 | 37281157 | 38897220 | Hay Fever, Asthma, Childhood onset asthma | 1, 2 |
| 17 | 40338997 | 40450012 | Asthma, Childhood onset asthma | 1, 2 |
| 17 | 43156023 | 43457886 | Hay Fever, Asthma | 1 |
| 17 | 45805811 | 45873184 | childhood onset asthma | 2 |
| 17 | 47299789 | 47481374 | Hay Fever, Asthma, shared adult and childhood onset asthma | 1, 2 |
| 18 | 48482900 | 48662349 | Hay Fever | 1 |
| 18 | 51781019 | 52366730 | Hay Fever, Asthma | 1 |
| 18 | 60005046 | 60018206 | Hay Fever, Childhood onset asthma | 1, 2 |
| 18 | 61412756 | 61627300 | Asthma, Childhood onset asthma | 1, 2 |
| 19 | 1149092 | 1171213 | childhood onset asthma | 2 |
| 19 | 33718053 | 33736279 | Hay Fever, Asthma, shared adult and childhood onset asthma | 1, 2 |
| 19 | 46219145 | 46370381 | Asthma | 1 |
| 20 | 45232161 | 45716594 | Hay Fever | 1 |
| 20 | 52168159 | 52268995 | Hay Fever | 1 |
| 20 | 62270637 | 62400021 | Hay Fever, Asthma | 1 |
| 21 | 36421331 | 36507786 | Asthma, shared adult and childhood onset asthma | 1, 2 |
| 22 | 37319425 | 37319425 | Hay Fever | 1 |
| 22 | 41798520 | 41941243 | Asthma | 1 |

¹All coordinates are hg19
²Johansson et al.: n = 41,926 asthma cases and 239,733 controls; 84,034 hay fever + eczema cases and 239,733 controls. Pividori et al.: n = childhood onset asthma 9,443 cases and 318,237 controls; 21,564 adult onset asthma cases and 318,237 controls.
³1 = A. Johansson, M. Rask-Andersen, T. Karlsson, W. E. Ek, Genome-wide association analysis of 350000 Caucasians from the UK Biobank identifies novel loci for asthma, hay fever and eczema. Hum Mol Genet 28, 4022-4041 (2019), 2 = M. Pividori, N. Schoettler, D. L. Nicolae, C. Ober, H. K. Im, Shared and distinct genetic risk factors for childhood-onset and adult-onset asthma: genome-wide and transcriptome-wide studies. Lancet Respir Med 7, 509-522 (2019).

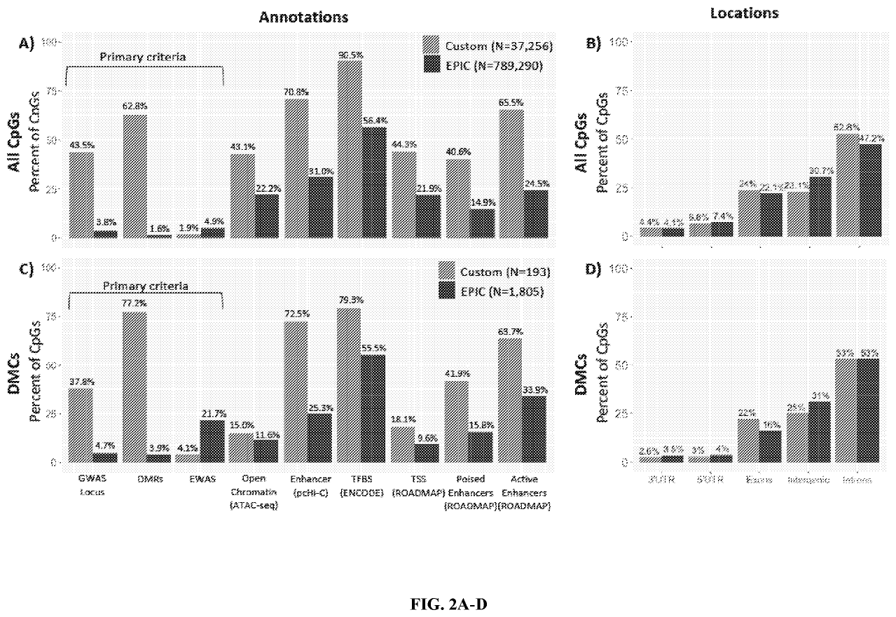

The distributions of CpGs on the Custom and EPIC arrays are shown by annotation category in FIG. 2A. Of note, 90% of the Custom array CpGs overlapped with a transcription factor binding site (TFBS) and 94% overlapped with a predicted enhancer, compared to 56% and 54% of the EPIC array CpGs, respectively (Fisher exact test [FET]p<10⁻¹⁶ for all tests). Moreover, compared to EPIC CpGs, Custom CpGs were enriched in introns (52.8% vs. 47.2%) and exons (24.0% vs. 22.1%) and depleted in intergenic regions (23.1% vs. 30.7%) and 5′UTRs (6.8% vs. 7.4%) (FET p<10⁻⁴ in all analyses) (FIG. 2B). The CpGs on the Custom array proportionally represented DMR-CpGs from WGBS studies in the African Americans, European Americans, and the combined samples, and from the WGBS studies and previous GWAS of asthma or allergic diseases; very few previous EWAS CpGs were included on the Custom array because nearly all were on the EPIC array (FIG. 6).

### Evaluating Methylation Patterns of CpGs on the Custom Array

To assess the reproducibility of methylation measures of CpGs on the Custom array, p values in airway epithelial cell DNA in the WGBS were compared to the same CpGs on the Custom and the EPIC arrays for the subset of individuals with all three measures (19 of the URECA children and 17 of the COAST young adults). CpGs were included with at least 10 WGBS reads that overlapped with an array CpG that passed QC. This resulted in 502,229 CpGs for the WGBS and EPIC array comparison in URECA and 24,744 and 20,861 CpGs for the WGBS and Custom array comparison in URECA and COAST, respectively. The β distribution plots were similar and highly correlated between CpGs measured by WGBS and the arrays (Spearman's p<2.2×10⁻¹⁶ for each) (FIG. 7). Notably, however, compared to the EPIC array, CpGs on the Custom array were depleted for hypermethylated CpGs and enriched for intermediate methylated (IM) CpGs.

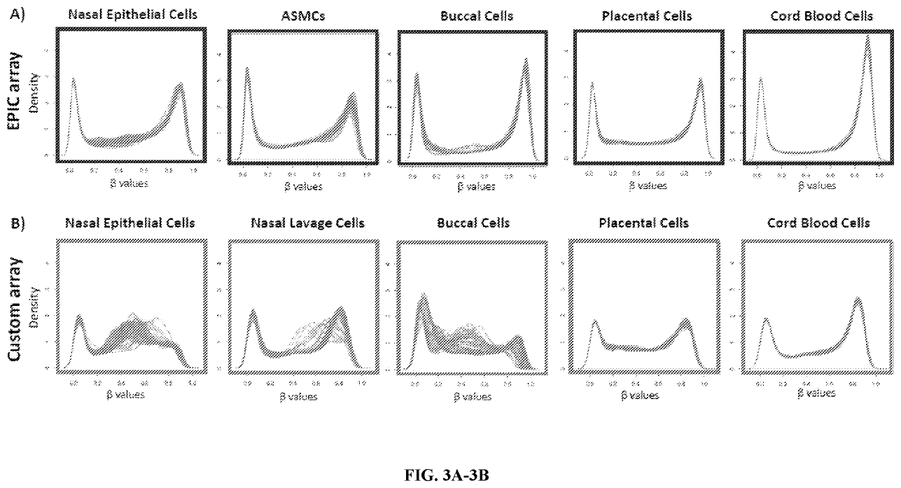

To more broadly assess the β distributions of CpGs on the EPIC and Custom arrays, they were compared across different tissues using available data for the EPIC array in DNA from airway epithelial cells (this study), airway smooth muscle cells (E. E. Thompson et al., Cytokine-induced molecular responses in airway smooth muscle cells inform genome-wide association studies of asthma. Genome Med 12, 64 (2020), and buccal cells, placenta cells, cord blood, and for the Custom array in DNA from nasal epithelial cells and nasal lavage cells from URECA children, and buccal cells, cord blood cells, or placenta tissue from infants in the Vitamin C to Decrease the Effects of Smoking in Pregnancy on Infant Lung Function (VCSIP) cohort (L. E. Shorey-Kendrick et al., Impact of vitamin C supplementation on placental DNA methylation changes related to maternal smoking: association with gene expression and respiratory outcomes. Clin Epigenetics 13, 177 (2021)). The β distributions were similar across cell sources for CpGs on the EPIC array (FIG. 3A) but showed varying patterns between cell types on the Custom array (FIG. 3B). Whereas an average of 68% (range: 60-79%) of CpGs on the EPIC array were either hypomethylated (0-20%) or hypermethylated (80-100%) in all six cell types, most on the Custom array (66% in nasal epithelial cells and 52% on average across cell types) were IM CpGs (β values between 20-80%) with depletions of CpGs with β values between both 0-10% (24% in nasal epithelial cells and 29% on average) and 90-100% (10% in nasal epithelial cells and 20% on average). In fact, the β value distributions of CpGs on the EPIC array in nine GTEx tissues are remarkably similar to those reported here.

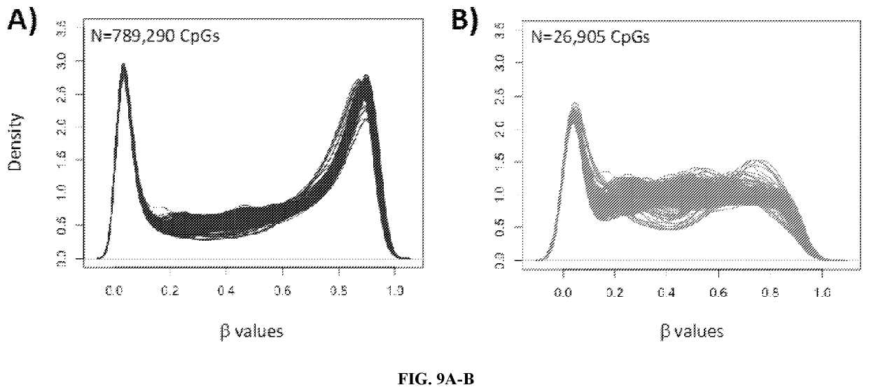

It was next asked whether the enrichment for IM CpGs was attributable to the selection criteria we used for designing the Custom array. To address this question, the CpGs on the EPIC array were filtered using the same pipeline as that used for selecting CpGs for the Custom array. These are CpGs that met criteria for inclusion on the Custom array but were excluded because they were on the EPIC array. These are referred to as “filtered EPIC” CpGs. Of the 789,290 CpGs on the EPIC array that passed QC (see Methods), 26,905 (3.4%) were designated as “high-value”. The β distribution of the filtered EPIC CpGs in nasal epithelial cells revealed a pattern similar to CpGs on the Custom array, with the majority (61%) having β values between 20-80% (FIG. 9). These results confirmed that selecting CpGs with prior evidence of disease association and overlap with chromatin marks of gene regulatory regions results in a depletion of both hypomethylated and hypermethylated CpGs and an enrichment for IM CpGs, are more variable across cell types compared to fully methylated and fully unmethylated CpGs.

### Ewas of Allergic Sensitization in 280 Multi-Ancestry Children Using the Custom and EPIC Arrays

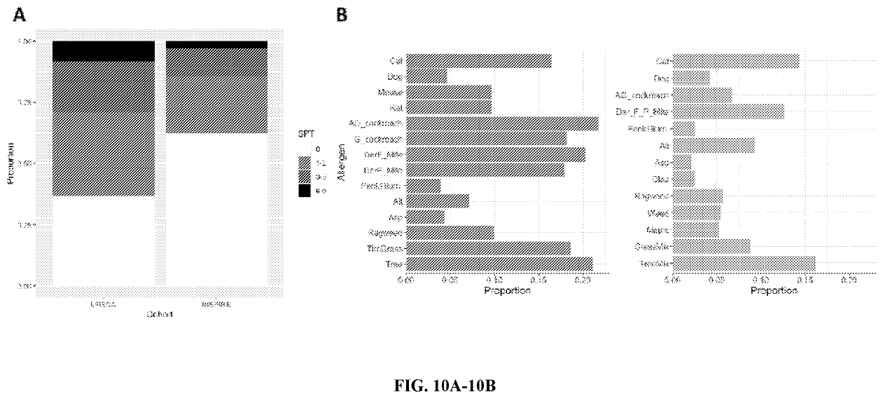

Experiments were conducted during development of embodiments herein to develop DNA methylation arrays that could detect asthma- or allergy-associated differential methylation that is missed by the content on the EPIC array. Allergic sensitization (atopy) is an IgE response to allergens (15) and a crucial step in the development of allergic diseases and asthma (16, 17). An EWAS of allergic sensitization (AS) was conducted in airway epithelial cell DNA collected from 280 11-year old URECA children using the Custom and the EPIC arrays. AS was defined as the percent positive skin prick tests (SPTs) to 14 airborne and oral allergens (see Methods). The demographic and clinical description of the URECA children is shown in Table 4 and FIG. 10. See Methods for further descriptions of the sample, the allergens tested, and the processing and QC of the array data.

TABLE 4
Demographic characteristics of URECA and INSPIRE samples.

| Characteristic | URECA | INSPIRE |
|---|---|---|
| Sample size (N) | 280 | 474 |
| Mean Age (range), years | 11 (11-12) | 6.3 (5.0-7.9) |
| Female sex | 46.8% | 47.0% |

Ethnicity

| | | |
|---|---|---|
| Non-Hispanic White | <1% | 65.4% |
| Non-Hispanic Black | 71.8% | 18.1% |
| Hispanic | 20.0% | 9.5% |
| Other | 7.9% | 7.6% |

Clinical Definitions

| | | |
|---|---|---|
| Asthma | 32.1% | 9.5% |
| Allergic asthma | 22.1% | 4.6% |
| Allergic sensitization (≥1 + SPT) | 63.2% | 37.8% |
| Non-Allergic/Non-Asthma | 27.1% | 62.2% |

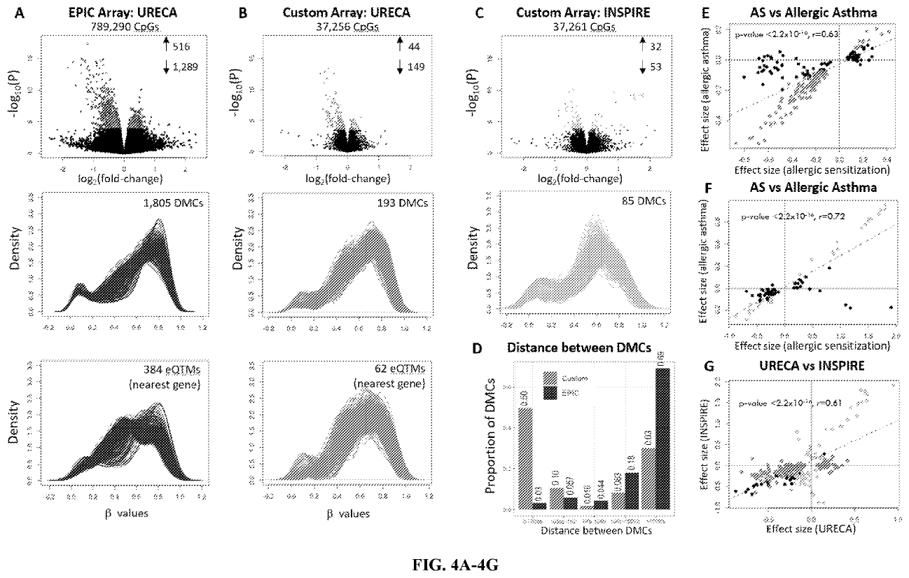

Two EWAS of AS were performed in the same URECA children using CpGs on the EPIC or Custom array. In each analysis, sex, percent epithelial cells, the first β ancestry PCs, and latent factors were included as covariates to adjust for additional unwanted variation (18, 19) (see Methods). Using a q-value threshold of 0.05, the EWAS of AS revealed 1,805 DMCs using the EPIC array and 193 DMCs using the Custom array (FIG. 4A-B). Overall, the Custom array was enriched for AS DMCs compared to the EPIC array (0.50% vs. 0.23%). Among the DMCs on the EPIC array, 295 (16%) were filtered EPIC CpGs, a significant enrichment compared to 3.5% of filtered CpGs among all CpGs on the array (FET p<2.2×10⁻¹⁶). In contrast to the different β distributions of CpGs on the EPIC and Custom arrays (FIG. 3A), the β distributions of DMCs from the two arrays were similar (FIG. 4A-B; middle panels), showing a near complete depletion of both hypomethylated and hypermethylated CpGs. These data further establish that AS DMCs are highly enriched for EVI CpGs in airway epithelial cells.

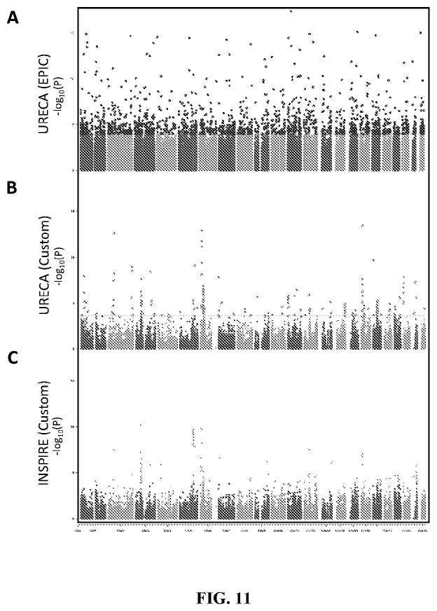

The DMCs on both arrays were distributed across the autosomes (FIG. 11A-B). However, whereas the DMCs on the Custom array show spikes of association signals, the DMCs on the EPIC array are more solitary and sparsely distributed across the genome. In fact, 50% of DMCs on the Custom array are within 100 bp of the next nearest DMC compared to only 3% of DMCs on the EPIC. Among the latter, 69% of DMCs are >100 kb from the next nearest DMC compared to only 30% on the Custom arrays. (FIG. 4D)

Most of the functional annotation categories were represented by DMCs proportionally to all CpGs on each array (FIG. 2A-B). However, DMCs from both arrays were depleted among transcription start sites compared to all CpGs on the arrays (Custom 18.1% vs. 44.3%; FET p=1.14×10⁻⁷; EPIC 9.6% vs 21.9%; FET p<2.2×10⁻¹⁶) and in areas of open chromatin on (Custom 15.0% vs. 43.1%; FET p=4.11×10⁻⁹; EPIC 11.6% vs. 22.2%; FET p<2.2×10⁻¹⁶). Among the primary criteria (prior evidence), CpGs in DMRs were marginally enriched for DMCs compared to all CpGs on the Custom array (77.2% vs. 62.8%; FET p=0.066) but significantly enriched on the EPIC array (3.9% vs. 1.6%; FET p=1.54×10⁻⁶). Similarly, DMCs in prior EWAS studies of asthma and allergic diseases were modestly enriched on the Custom array (4.1% vs. 1.9%; FET p=0.059) and significantly enriched on the EPIC array (21.7% vs. 4.9%; FET p<2.2×10⁻¹⁶). In contrast, CpGs at GWAS loci for asthma and allergic diseases were not enriched among DMCs on the Custom array (37.8% vs 43.5%; FET p=0.32) and only modestly enriched on the EPIC array (4.7% vs 3.8%; FET p=0.042). The locations of the DMCs were proportional to all CpGs on the arrays.

Because AS is an important step in the development of childhood asthma, experiments were conducted during development of embodiments herein to determine whether AS DMCs were also associated with allergic asthma. Considering only the AS DMCs on the EPIC array, 1,155 (64%) were also associated with allergic asthma at q-value <0.05; among the AS DMCs on the Custom array, 115 (60%) were also associated with allergic asthma at q-value <0.05. The effect sizes were significantly correlated between AS DMCs and allergic asthma DMCs (r=0.68) (FIG. 4E). Among the CpGs that were DMCs for both AS and allergic asthma, all showed the same direction of effect; among all AS DMCs, 93% showed the same direction of effect with allergic asthma (r=0.84)

### Validating the Custom Array in the INSPIRE Cohort

The URECA cohort is diverse with respect to ancestry but includes <1% non-Hispanic white children. Therefore, to both replicate results of the EWAS in URECA children and assess the performance of the Custom array in a primarily non-Hispanic white population, 5- to 7-year old children from the Infant Susceptibility to Pulmonary Infections and Asthma Following RSV Exposure (INSPIRE) study were studied (20). INSPIRE mothers were enrolled during pregnancy into this population-based longitudinal study in central Tennessee. An EWAS was performed using the Custom array and nasal epithelial cell DNA from 474 children with measures of AS (proportion of positive SPT to 13 inhaled allergens). The demographic and clinical characteristics of the INSPIRE children are shown in Table 4. Studies with the EPIC array were not performed in this cohort.

The AS EWAS model in INSPIRE included as covariates sex, parent-reported race or ethnicity, DNA concentration, and latent factors to adjust for unwanted variation (18) (see Methods). At a q-value threshold of <0.05, 85 CpGs on the Custom array were DMCs (0.2% of CpGs); the beta distributions of the DMCs were similar to those observed in URECA (FIG. 4C). As in the URECA Custom array EWAS, there were spikes of association signals in the INSPIRE EWAS, many at shared regions (FIG. 11). Among the AS DMCs in INSPIRE, 38% were also associated with allergic asthma, all with the same direction of effect; among all AS DMCs in INSPIRE, 93% had the same direction of effect with allergic asthma (FIG. 4F). Moreover, among the AS DMCs in URECA, 25 were also AS DMCs in INSPIRE at the 0.05 FDR threshold and all had concordant directions of effect. The 253 CpGs that were DMCs in either the URECA or INSPIRE AS EWAS at FDR <0.05 were highly correlated (r=0.61; P<2.2×10⁻¹⁶) and 82% had concordant directions of effect (FIG. 4G). The high reproducibility of EWAS results in two cohorts demonstrates that the high-value CpGs on the Custom array identify AS DMCs that are robust to ancestry, ascertainment strategies, age at sampling, and geography.

### Evaluating the Biological Significance of DMCs in Airway Epithelial Cells

Nearly all CpGs on the Custom array overlap with a TFBS or enhancer mark. Accordingly, it was tested whether these CpGs are correlated with the expression of genes in airway epithelial cells more often than CpGs on the EPIC array (G. Elliott et al., Intermediate DNA methylation is a conserved signature of genome regulation. Nat Commun 6, 6363 (2015). To test this, gene expression data was used (M. C. Altman et al., Endotype of allergic asthma with airway obstruction in urban children. J Allergy Clin Immunol 10.1016/j.jaci.2021.02.040 (2021)) in the same cells as those used for DNA methylation studies in 249 of the URECA children and defined two sets of genes from among the 15,551 genes detected as expressed in these cells: the nearest gene to each CpG and the promoter capture Hi-C (pcHi-C)-defined target gene in airway epithelial cells (see Methods). The latter identified putative enhancers that physically interact with target gene promoters and account for the 3-dimensional structure of the genome that allows for distal regulatory elements to interact with and regulate the activity of promoters over large distances (e.g., up to 1 Mb or more), which are often not the nearest gene. Among the nearest expressed genes to DMCs on the Custom (98 genes) and EPIC (1,449 genes) arrays, 63 were the nearest gene on both arrays. Among the pcHi-C target genes for DMCs on the Custom (245 genes) or EPIC (1,155 genes), 95 were target genes on both arrays. Thus, although there were no overlapping CpGs between the arrays, 101 of nearest or pcHi-C target genes were shared on both arrays (Table 5). The 318 nearest or pcHi-C target genes on the Custom array that were expressed in airway epithelial cells were enriched in 16 pathways (FDR <0.05), including “Th1 and Th2 cell differentiation”, “JAK-STAT signaling”, “Th17 cell differentiation”, and “Viral protein interaction with cytokine and cytokine receptor” (Table 6). In contrast, the 2,366 nearest or pcHi-C target genes on the EPIC array that were expressed in airway epithelial cells were not enriched for any pathways (smallest FDR=0.13). These findings further demonstrate that the CpGs on the Custom array are enriched in pathways relevant to asthma and allergic disease.

TABLE 5
Nearest and pcHi-C target genes for AS DMCs on the EPIC and Custom arrays in URECA. For all DMCs
on the EPIC and Custom arrays, a nearest gene and a pcHi-C target gene (if identified) were assigned
(see Methods). The prior evidence category for each CpG is also shown (GWAS, EWAS, DMR).

| | Nearest Genes | | | | pcHi-C genes | | | |
|---|---|---|---|---|---|---|---|---|
| Gene | Custom | | EPIC | | Custom | | EPIC | |
| name | CpG | Evidence | CpG | Evidence | CpG | Evidence | CpG | Evidence |
| ABCC5 | cg05910779_BC21 | DMR | | | | | cg01966760 | NA |
| ABCC5 | cg05910778_BC21 | DMR | | | | | cg16221425 | NA |
| ABHD14A | | | | | cg07095346_BC21 | EWAS | cg22988305 | EWAS |
| ACBD4 | | | | | cg22600575_BC21 | GWAS | cg05257097 | GWAS |
| ACBD4 | | | | | cg22600571_TC21 | GWAS | | |
| ACBD4 | | | | | cg22600572_BC21 | GWAS | | |
| ADAMTS15 | | | cg18023339 | NA | cg17084526_TC21 | DMR | cg21770200 | EWAS |
| AHNAK2 | cg20048107_TC21 | DMR | | | | | cg01177261 | DMR |
| AHNAK2 | cg20048043_BC21 | DMR | | | | | | |
| ALDOA | | | | | cg21355554_BC21 | DMR | cg04665974 | NA |
| ALDOA | | | | | cg21355555_TC21 | DMR | | |
| ALOX15 | cg22082619_BC21 | GWAS, | cg19595239 | GWAS, | | | | |
| | | DMR | | EWAS | | | | |
| ALOX15 | | | cg17389538 | GWAS | | | | |
| ANKRD 10 | | | cg27461824 | EWAS | cg19162921_BC21 | DMR | | |
| ANO1 | cg12357484_TC21 | EWAS | cg25723217 | EWAS | | | | |
| ANO1 | | | cg12849969 | NA | | | | |
| ANO1 | | | cg24308267 | NA | | | | |
| ARHGAP27 | | | cg22153994 | EWAS | cg22601529_BC21 | GWAS, | cg21137244 | GWAS, |
| | | | | | | DMR | | EWAS |
| ARHGAP27 | | | | | cg22601528_TC21 | GWAS, | | |
| | | | | | | DMR | | |
| ARHGEF3 | cg04967825_TC21 | DMR | cg01016119 | DMR, | cg04967816_TC21 | DMR | | |
| | | | | EWAS | | | | |
| ARHGEF3 | cg04967816_TC21 | DMR | cg13068706 | NA | | | | |
| ARHGEF3 | cg04967826_BC21 | DMR | | | | | | |
| ARHGEF7 | | | cg20847766 | EWAS | cg19162921_BC21 | DMR | cg27461824 | EWAS |
| ATP8B1 | | | cg02550398 | NA | cg23606851_BC21 | DMR | cg18475483 | DMR |
| ATP8B1 | | | | | cg23606849_TC21 | DMR | cg02550398 | NA |
| ATP8B1 | | | | | cg23606852_BC21 | DMR | cg24331818 | DMR |
| BLOC1S6 | | | | | cg20334662_BC21 | DMR | cg13570892 | EWAS |
| BLOC1S6 | | | | | | | cg07558734 | DMR, |
| | | | | | | | | EWAS |
| C12orf57 | | | | | cg17241870_BC21 | DMR | cg16707011 | NA |
| C14orf79 | | | cg01177261 | DMR | cg20048043_BC21 | DMR | | |
| C14orf79 | | | | | cg20048107_TC21 | DMR | | |
| C15orf48 | | | cg14661236 | NA | cg20334662_BC21 | DMR | | |
| C3orf18 | | | | | cg04902046_TC21 | DMR | cg09575750 | NA |
| C3orf18 | | | | | cg04902043_BC21 | DMR | | |
| CAPN14 | cg02630973_BC21 | DMR | cg04132353 | DMR, | | | | |
| | | | | EWAS | | | | |
| CAPN14 | | | cg01827910 | NA | | | | |
| CCNI2 | | | | | cg08524768_BC21 | GWAS | cg16525542 | GWAS |
| CCNI2 | | | | | cg08524766_BC21 | GWAS | cg23475112 | GWAS, |
| | | | | | | | | EWAS |
| CDCA4 | | | | | cg20048043_BC21 | DMR | cg01177261 | DMR |
| CDCA4 | | | | | cg20050618_BC21 | DMR | | |
| CDCA4 | | | | | cg20048107_TC21 | DMR | | |
| CDK2 | | | | | cg17662800_BC21 | GWAS | cg17865265 | GWAS |
| CDK2 | | | | | | | cg08362736 | GWAS |
| CEP72 | | | | | cg07553021_TC21 | DMR | cg00049323 | EWAS |
| CISH | cg04902026_TC21 | DMR | cg09575750 | NA | | | | |
| CISH | cg04902028_TC21 | DMR | cg23005227 | DMR, | | | | |
| | | | | EWAS | | | | |
| CISH | cg04902029_BC21 | DMR | cg16315329 | EWAS | | | | |
| CISH | cg04902032_TC21 | DMR | | | | | | |
| CISH | cg04902033_BC21 | DMR | | | | | | |
| CISH | cg04902034_BC21 | DMR | | | | | | |
| CISH | cg04902035_BC21 | DMR | | | | | | |
| CISH | cg04902037_BC21 | DMR | | | | | | |
| CISH | cg04902041_BC21 | DMR | | | | | | |
| CISH | cg04902043_BC21 | DMR | | | | | | |
| CISH | cg04902046_TC21 | DMR | | | | | | |
| CLEC16A | cg21129219_BC21 | GWAS, | cg10364862 | GWAS, | | | | |
| | | DMR | | EWAS | | | | |
| CLEC16A | cg21129001_TC21 | GWAS | | | | | | |
| CLEC16A | cg21129004_TC21 | GWAS | | | | | | |
| CLEC16A | cg21129010_BC21 | GWAS | | | | | | |
| CLEC16A | cg21129002_BC21 | GWAS | | | | | | |
| CLEC16A | cg21129003_BC21 | GWAS | | | | | | |
| CLEC16A | cg21129011_BC21 | GWAS | | | | | | |
| CLEC16A | cg21129012_TC21 | GWAS | | | | | | |
| CLMP | cg17016940_BC21 | DMR | cg01941390 | NA | | | | |
| CLMP | | | cg16343910 | NA | | | | |
| CLMP | | | cg25379149 | NA | | | | |
| COASY | | | | | cg22547525_TC21 | GWAS | cg06848514 | NA |
| COASY | | | | | | | cg02691389 | EWAS |
| COASY | | | | | | | cg17177779 | NA |
| CTSC | cg16735276_BC21 | DMR | cg08522340 | DMR, | | | | |
| | | | | EWAS | | | | |
| CTSC | | | cg16118839 | DMR, | | | | |
| | | | | EWAS | | | | |
| CTSC | | | cg09706192 | DMR | | | | |
| CYB561D2 | | | | | cg04902046_TC21 | DMR | cg15152301 | NA |
| CYB561D2 | | | | | cg04902043_BC21 | DMR | cg09575750 | NA |
| CYB561D2 | | | | | | | | |
| CYB561D2 | | | | | | | | |
| CYP11A1 | cg22186216_TC21 | EWAS | cg25788983 | NA | | | | |
| DCAKD | | | | | cg22596680_TC21 | GWAS | cg00146864 | GWAS, |
| | | | | | | | | DMR, |
| | | | | | | | | EWAS |
| DCAKD | | | | | cg22601159_TC21 | GWAS | cg00897875 | GWAS, |
| | | | | | | | | DMR, |
| | | | | | | | | EWAS |
| DCAKD | | | | | cg22601346_TC21 | GWAS, | cg20864568 | GWAS, |
| | | | | | | DMR | | DMR, |
| | | | | | | | | EWAS |
| DCAKD | | | | | cg22601347_TC21 | GWAS, | cg23315838 | NA |
| | | | | | | DMR | | |
| DEF6 | | | | | cg09420574_TC21 | DMR | cg09649521 | NA |
| EFTUD2 | | | cg24508472 | NA | cg22596680_TC21 | GWAS | | |
| EIF2B5 | | | cg20935483 | NA | cg05910779_BC21 | DMR | cg01966760 | NA |
| EIF2B5 | | | cg14141843 | EWAS | cg05910778_BC21 | DMR | cg16221425 | NA |
| ERBB2 | | | cg14377681 | GWAS, | cg22513183_BC21 | GWAS | | |
| | | | | EWAS | | | | |
| EXOC3 | cg07553021_TC21 | DMR | cg00049323 | EWAS | cg07553021_TC21 | DMR | cg00049323 | EWAS |
| FMNL1 | | | | | cg22601346_TC21 | GWAS, | cg00146864 | GWAS, |
| | | | | | | DMR | | DMR, |
| | | | | | | | | EWAS |
| FMNL1 | | | | | cg22601347_TC21 | GWAS, | cg00897875 | GWAS, |
| | | | | | | DMR | | DMR, |
| | | | | | | | | EWAS |
| FMNL1 | | | | | cg22601529_BC21 | GWAS, | cg20864568 | GWAS, |
| | | | | | | DMR | | DMR, |
| | | | | | | | | EWAS |
| FMNL1 | | | | | cg22601159_TC21 | GWAS | cg21137244 | GWAS, |
| | | | | | | | | EWAS |
| FMNL1 | | | | | cg22596680_TC21 | GWAS | | |
| FMNL1 | | | | | cg22601528_TC21 | GWAS, | | |
| | | | | | | DMR | | |
| FOXP1 | cg05089320_TC21 | DMR | cg06262288 | NA | | | | |
| GCNT2 | cg09150434_TC21 | DMR | cg25531743 | DMR | | | | |
| GCNT2 | cg09150474_TC21 | DMR | | | | | | |
| GCNT2 | cg09150429_TC21 | DMR | | | | | | |
| GCNT2 | cg09150432_TC21 | DMR | | | | | | |
| GCNT2 | cg09150433_BC21 | DMR | | | | | | |
| GCNT2 | cg09150431_BC21 | DMR | | | | | | |
| GCNT2 | cg09150428_TC21 | DMR | | | | | | |
| GDF9 | | | | | cg08524766_BC21 | GWAS | cg23475112 | GWAS, |
| | | | | | | | | EWAS |
| GDF9 | | | | | cg08524768_BC21 | GWAS | | |
| GNAI2 | | | | | cg04902043_BC21 | DMR | cg14433598 | NA |
| GNAI2 | | | | | cg04902046_TC21 | DMR | cg09579833 | EWAS |
| GNG12 | cg00902711_TC21 | DMR | cg19277299 | NA | | | cg19277299 | NA |
| GRB7 | | | | | cg22513183_BC21 | GWAS | cg14377681 | EWAS, |
| | | | | | | | | GWAS |
| H6PD | | | cg09869882 | NA | cg00185221_TC21 | GWAS | | |
| HEXIM1 | | | | | cg22596680_TC21 | GWAS | cg00146864 | GWAS, |
| | | | | | | | | DMR, |
| | | | | | | | | EWAS |
| HEXIM1 | | | | | cg22601346_TC21 | GWAS, | cg00897875 | GWAS, |
| | | | | | | DMR | | DMR, |
| | | | | | | | | EWAS |
| HEXIM1 | | | | | cg22601347_TC21 | GWAS, | cg20864568 | GWAS, |
| | | | | | | DMR | | DMR, |
| | | | | | | | | EWAS |
| HEXIM1 | | | | | cg22601528_TC21 | GWAS, | cg21137244 | GWAS, |
| | | | | | | DMR | | EWAS |
| HEXIM1 | | | | | cg22601529_BC21 | GWAS, | | |
| | | | | | | DMR | | |
| HYAL1 | | | | | cg04902046_TC21 | DMR | cg09575750 | NA |
| HYAL1 | | | | | cg04902043_BC21 | DMR | | |
| INO80 | | | | | cg20291832_BC21 | GWAS | cg25454569 | NA |
| IRF1 | | | | | cg08524766_BC21 | GWAS | cg23475112 | GWAS, |
| | | | | | | | | EWAS |
| IRF1 | | | | | cg08524768_BC21 | GWAS | | |
| ITIH4 | cg07095346_BC21 | EWAS | | | | | cg09469170 | EWAS |
| JAZF1 | cg10813825_TC21 | GWAS | cg04266607 | GWAS | | | | |
| JAZF1 | | | cg06607889 | NA | | | | |
| KDM8 | | | | | cg21317192_TC21 | GWAS | cg07057349 | NA |
| KIF18B | | | | | cg22601346_TC21 | GWAS, | cg00146864 | GWAS, |
| | | | | | | DMR | | DMR, |
| | | | | | | | | EWAS |
| KIF18B | | | | | cg22596680_TC21 | GWAS | cg00897875 | GWAS, |
| | | | | | | | | DMR, |
| | | | | | | | | EWAS |
| KIF18B | | | | | cg22601347_TC21 | GWAS, | cg20864568 | GWAS, |
| | | | | | | DMR | | DMR, |
| | | | | | | | | EWAS |
| LRRFIP1 | cg04356823_TC21 | DMR | cg02797113 | DMR, | | | cg16630940 | EWAS |
| | | | | EWAS | | | | |
| LRRFIP1 | cg04356831_BC21 | DMR | cg16630940 | EWAS | | | | |
| LRRFIPI | cg04356833_BC21 | DMR | | | | | | |
| LRRFIP1 | | | | | | | | |
| LRRFIP1 | | | | | | | | |
| LRRFIP1 | | | | | | | | |
| LY96 | cg12730697_BC21 | DMR | cg22007804 | NA | | | cg22007804 | NA |
| LY96 | cg12730695_TC21 | DMR | | | | | | |
| MAD1L1 | cg10556822_BC21 | DMR | cg18752987 | NA | cg10556822_BC21 | DMR | | |
| MANF | | | | | cg04902046_TC21 | DMR | cg23005227 | DMR, |
| | | | | | | | | EWAS |
| MANF | | | | | cg04902026_TC21 | DMR | | |
| MANF | | | | | cg04902033_BC21 | DMR | | |
| MANF | | | | | cg04902034_BC21 | DMR | | |
| MANF | | | | | cg04902037_BC21 | DMR | | |
| MANF | | | | | cg04902041_BC21 | DMR | | |
| MANF | | | | | cg04902035_BC21 | DMR | | |
| MANF | | | | | cg04902028_TC21 | DMR | | |
| MANF | | | | | cg04902032_TC21 | DMR | | |
| MANF | | | | | cg04902029_BC21 | DMR | | |
| MANF | | | | | cg04902043_BC21 | DMR | | |
| MAP3K14 | cg22600571_TC21 | GWAS | cg00146864 | GWAS, | cg22601159_TC21 | GWAS | | |
| | | | | DMR, | | | | |
| | | | | EWAS | | | | |
| MAP3K14 | cg22600572_BC21 | GWAS | cg00897875 | GWAS, | | | | |
| | | | | DMR, | | | | |
| | | | | EWAS | | | | |
| MAP3K14 | cg22600575_BC21 | GWAS | cg05257097 | GWAS | | | | |
| MAP3K14 | cg22601159_TC21 | GWAS | cg16022555 | GWAS | | | | |
| MAP3K14 | cg22601346_TC21 | GWAS, | cg20864568 | GWAS, | | | | |
| | | DMR | | DMR, | | | | |
| | | | | EWAS | | | | |
| MAP3K14 | cg22601347_TC21 | GWAS, | cg21137244 | GWAS, | | | | |
| | | DMR | | EWAS | | | | |
| MAP3K14 | cg22601528_TC21 | GWAS, | | | | | | |
| | | DMR | | | | | | |
| MAP3K14 | cg22601529_BC21 | GWAS, | | | | | | |
| | | DMR | | | | | | |
| MMP19 | cg17662800_BC21 | GWAS | cg17865265 | GWAS | | | | |
| MMP19 | | | cg08362736 | GWAS | | | | |
| MRPS6 | cg25807822_BC21 | DMR | cg25011666 | NA | | | | |
| MRPS6 | | | cg21291385 | EWAS | | | | |
| MYC | cg13109596_TC21 | GWAS | cg08349436 | NA | | | | |
| MYC | | | cg03691530 | EWAS | | | | |
| MYC | | | cg21975232 | NA | | | | |
| MYC | | | cg26169156 | NA | | | | |
| NAT6 | | | | | cg04902029_BC21 | DMR | cg23005227 | DMR, |
| | | | | | | | | EWAS |
| NAT6 | | | | | cg04902046_TC21 | DMR | | |
| NAT6 | | | | | cg04902034_BC21 | DMR | | |
| NAT6 | | | | | cg04902028_TC21 | DMR | | |
| NAT6 | | | | | cg04902032_TC21 | DMR | | |
| NAT6 | | | | | cg04902043_BC21 | DMR | | |
| NAT6 | | | | | cg04902033_BC21 | DMR | | |
| NAT6 | | | | | cg04902037_BC21 | DMR | | |
| NAT6 | | | | | cg04902035_BC21 | DMR | | |
| NAT6 | | | | | cg04902026_TC21 | DMR | | |
| NAT6 | | | | | cg04902041_BC21 | DMR | | |
| NEDD4L | cg23606849_TC21 | DMR | cg18475483 | DMR | | | | |
| NEDD4L | cg23606851_BC21 | DMR | cg24331818 | DMR | | | | |
| NEDD4L | cg23606852_BC21 | DMR | | | | | | |
| NR1D1 | | | | | cg22515799_TC21 | GWAS, | cg13762512 | GWAS |
| | | | | | | DMR | | |
| NR1D1 | | | | | cg22515797_BC21 | GWAS | | |
| NRIP1 | cg21021629_BC21 | EWAS | cg00712106 | EWAS | | | | |
| OIP5 | | | | | cg20291832_BC21 | GWAS | cg25454569 | NA |
| P4HA2 | | | cg16476284 | NA | cg08524766_BC21 | GWAS | cg23475112 | GWAS, |
| | | | | | | | | EWAS |
| P4HA2 | | | | | cg08524768_BC21 | GWAS | | |
| PARL | | | cg01966760 | NA | cg05910779_BC21 | DMR | | |
| PARL | | | cg16221425 | NA | cg05910778_BC21 | DMR | | |
| PELP1 | | | | | cg22082619_BC21 | GWAS, | cg17389538 | GWAS |
| | | | | | | DMR | | |
| PELP1 | | | | | | | cg19595239 | GWAS, |
| | | | | | | | | EWAS |
| PFKFB3 | | | cg22750548 | GWAS | cg14607001_TC21 | GWAS | cg04808066 | NA |
| PLCD3 | | | | | cg22601346_TC21 | GWAS, | cg20864568 | GWAS, |
| | | | | | | DMR | | DMR, |
| | | | | | | | | EWAS |
| PLCD3 | | | | | cg22596680_TC21 | GWAS | cg00897875 | GWAS, |
| | | | | | | | | DMR, |
| | | | | | | | | EWAS |
| PLCD3 | | | | | cg22601347_TC21 | GWAS, | | |
| | | | | | | DMR | | |
| PRDM10 | cg17084526_TC21 | DMR | cg25007761 | NA | cg17084526_TC21 | DMR | cg25007761 | NA |
| PRDM10 | | | cg06182390 | NA | | | | |
| PRKAG2 | cg11918680_TC21 | DMR | cg26405880 | NA | cg11918680_TC21 | DMR | cg09932376 | NA |
| PRKAG2 | | | cg04578183 | NA | | | | |
| PRKAG2 | | | cg09932376 | NA | | | | |
| RAB38 | | | cg00167102 | NA | cg16735276_BC21 | DMR | cg09706192 | DMR |
| RAB38 | | | | | | | cg08522340 | DMR. |
| | | | | | | | | EWAS |
| RAB38 | | | | | | | cg16118839 | DMR. |
| | | | | | | | | EWAS |
| RAD50 | | | | | cg08524768_BC21 | GWAS | cg23475112 | GWAS, |
| | | | | | | | | EWAS |
| RAD50 | | | | | cg08524766_BC21 | GWAS | | |
| RASSF1 | | | | | cg04902026_TC21 | DMR | cg09575750 | NA |
| RASSF1 | | | | | cg04902028_TC21 | DMR | cg23005227 | DMR, |
| | | | | | | | | EWAS |
| RASSF1 | | | | | cg04902029_BC21 | DMR | | |
| RASSF1 | | | | | cg04902032_TC21 | DMR | | |
| RASSF1 | | | | | cg04902033_BC21 | DMR | | |
| RASSF1 | | | | | cg04902034_BC21 | DMR | | |
| RASSF1 | | | | | cg04902035_BC21 | DMR | | |
| RASSF1 | | | | | cg04902037_BC21 | DMR | | |
| RMI2 | | | | | cg21129011_BC21 | GWAS | cg10364862 | GWAS, |
| | | | | | | | | EWAS |
| RMI2 | | | | | cg21129012_TC21 | GWAS | | |
| RMI2 | | | | | cg21129003_BC21 | GWAS | | |
| RMI2 | | | | | cg21129010_BC21 | GWAS | | |
| RMI2 | | | | | cg21129004_TC21 | GWAS | | |
| RMI2 | | | | | cg21129002_BC21 | GWAS | | |
| RMI2 | | | | | cg21129001_TC21 | GWAS | | |
| RPL7 | | | | | cg12730695_TC21 | DMR | cg18305583 | NA |
| RPL7 | | | | | cg12730697_BC21 | DMR | | |
| S100PBP | | | cg02962744 | NA | cg00549862_BC21 | DMR | | |
| S100PBP | | | | | cg00549865_BC21 | DMR | | |
| S100PBP | | | | | cg00549864_TC21 | DMR | | |
| SEPT8 | | | cg16525542 | GWAS | cg08524766_BC21 | GWAS | cg23475112 | GWAS, |
| | | | | | | | | EWAS |
| SEPT8 | | | | | cg08524768_BC21 | GWAS | | |
| SHROOM1 | | | | | cg08524766_BC21 | GWAS | cg23475112 | GWAS, |
| | | | | | | | | EWAS |
| SHROOM1 | | | | | cg08524768_BC21 | GWAS | | |
| SLC12A7 | | | | | cg07553021_TC21 | DMR | cg00049323 | EWAS |
| SLC22A5 | cg08524768_BC21 | GWAS | cg23475112 | GWAS, | | | cg16476284 | GWAS |
| | | | | EWAS | | | | |
| SLC22A5 | cg08524766_BC21 | GWAS | | | | | | |
| SLC25A39 | | | cg19481596 | NA | cg22547036_TC21 | GWAS | cg00897875 | GWAS, |
| | | | | | | | | DMR, |
| | | | | | | | | EWAS |
| SLC25A39 | | | | | cg22547038_BC21 | GWAS | cg20864568 | GWAS, |
| | | | | | | | | DMR, |
| | | | | | | | | EWAS |
| SLC25A39 | | | | | cg22601347_TC21 | GWAS, | | |
| | | | | | | DMR | | |
| SLC25A39 | | | | | cg22601346_TC21 | GWAS, | | |
| | | | | | | DMR | | |
| SLC48A1 | | | | | cg17561368_TC21 | DMR | cg15635287 | NA |
| SLC48A1 | | | | | cg17561366_TC21 | DMR | cg26115531 | EWAS |
| SLC48A1 | | | | | cg17561362_TC21 | DMR | | |
| SNX8 | cg10562002_BC21 | DMR | cg06047184 | DMR | | | | |
| SOCS1 | | | | | cg21129001_TC21 | GWAS | cg10364862 | GWAS, |
| | | | | | | | | EWAS |
| SOCS1 | | | | | cg21129010_BC21 | GWAS | | |
| SOCS1 | | | | | cg21129004_TC21 | GWAS | | |
| SOCS1 | | | | | cg21129003_BC21 | GWAS | | |
| SOCS1 | | | | | cg21129219_BC21 | GWAS, | | |
| | | | | | | DMR | | |
| SOCS1 | | | | | cg21129002_BC21 | GWAS | | |
| SOCS1 | | | | | cg21129011_BC21 | GWAS | | |
| SOCS1 | | | | | cg21129012_TC21 | GWAS | | |
| SPATA32 | | | | | cg22601346_TC21 | GWAS, | cg00897875 | GWAS, |
| | | | | | | DMR | | DMR, |
| | | | | | | | | EWAS |
| SPATA32 | | | | | cg22601347_TC21 | GWAS, | cg20864568 | GWAS, |
| | | | | | | DMR | | DMR, |
| | | | | | | | | EWAS |
| SPATA32 | | | | | | | cg00146864 | GWAS, |
| | | | | | | | | DMR, |
| | | | | | | | | EWAS |
| SSBP3 | cg00792117_TC21 | DMR | cg19682405 | NA | | | | |
| SSR3 | cg05713868_BC21 | DMR | | | | | cg20877312 | NA |
| SSR3 | cg05713870_BC21 | DMR | | | | | | |
| STAT3 | | | | | cg22547525_TC21 | GWAS | cg02691389 | EWAS |
| STAT3 | | | | | cg22547036_TC21 | GWAS | cg17177779 | NA |
| STAT3 | | | | | cg22547038_BC21 | GWAS | cg06848514 | NA |
| STON1 | cg02793110_BC21 | DMR | cg03390090 | NA | | | | |
| STON1 | cg02793111_BC21 | DMR | cg07150906 | DMR | | | | |
| STON1 | cg02793115_BC21 | DMR | cg10463553 | NA | | | | |
| STON1 | cg02793116_TC21 | DMR | cg23971565 | NA | | | | |
| STON1 | cg02793118_TC21 | DMR | | | | | | |
| STON1 | cg02793119_TC21 | DMR | | | | | | |
| SYNPO | | | cg25162888 | EWAS | cg08706842_TC21 | DMR | | |
| SYNPO | | | cg06675531 | EWAS | | | | |
| TAOK2 | | | | | cg21355555_TC21 | DMR | cg04665974 | NA |
| TAOK2 | | | | | cg21355554_BC21 | DMR | | |
| TMEM14C | | | | | cg09150474_TC21 | DMR | cg25531743 | DMR |
| TMEM54 | | | | | cg00549865_BC21 | DMR | cg02962744 | NA |
| TMEM54 | | | | | cg00549864_TC21 | DMR | | |
| TMEM54 | | | | | cg00549861_TC21 | DMR | | |
| TMEM54 | | | | | cg00549862_BC21 | DMR | | |
| TPPP | | | | | cg07553021_TC21 | DMR | cg00049323 | EWAS |
| TRERF1 | cg09496657_BC21 | DMR | cg19406053 | NA | | | | |
| TRIM69 | cg20334662_BC21 | DMR | cg09359575 | NA | | | | |
| TRPM8 | cg04308881_TC21 | DMR | cg10549071 | DMR, | cg04308881_TC21 | DMR | cg10549071 | DMR, |
| | | | | EWAS | | | | EWAS |
| TRPM8 | cg04308883_BC21 | DMR | cg20285660 | NA | cg04308883_BC21 | DMR | | |
| TRPM8 | | | | | | | | |
| TRPM8 | | | | | | | | |
| TRPM8 | | | | | | | | |
| TRPM8 | | | | | | | | |
| TRPM8 | | | | | | | | |
| TRPM8 | | | | | | | | |
| WIBG | | | | | cg17662800_BC21 | GWAS | cg08362736 | GWAS |
| WIBG | | | | | | | cg17865265 | GWAS |
| ZMYND10 | | | | | cg04902026_TC21 | DMR | cg09575750 | NA |
| ZMYND10 | | | | | cg04902028_TC21 | DMR | cg09579833 | EWAS |
| ZMYND10 | | | | | cg04902029_BC21 | DMR | cg23005227 | DMR, |
| | | | | | | | | EWAS |
| ZMYND10 | | | | | cg04902032_TC21 | DMR | | |
| ZMYND10 | | | | | cg04902033_BC21 | DMR | | |
| ZMYND10 | | | | | cg04902034_BC21 | DMR | | |
| ZMYND10 | | | | | cg04902035_BC21 | DMR | | |
| ZMYND10 | | | | | cg04902037_BC21 | DMR | | |
| ZMYND10 | | | | | cg04902041_BC21 | DMR | | |
| ZMYND10 | | | | | cg04902043_BC21 | DMR | | |
| ZMYND10 | | | | | cg04902046_TC21 | DMR | | |

TABLE 6
Results of KEGG pathway analysis on genes nearest DMCs and
pcHi-C target genes on the Custom array using iPathway Guide.
The list of genes (N = 318) was submitted to iPathway
Guide using the total list of genes nearest CpGs on the
Custom and EPIC arrays combined (N = 14,049) as background.
The “countAll” column lists the number of genes
in the background list that fall into the pathways shown.

| Pathway | countDE | countAll | fdr |
|---|---|---|---|
| Prolactin signaling pathway | 9 | 62 | 0.011 |
| Th1 and Th2 cell differentiation | 6 | 84 | 0.022 |
| Viral carcinogenesis | 13 | 164 | 0.022 |
| Adipocytokine signaling pathway | 8 | 55 | 0.022 |
| JAK-STAT signaling pathway | 11 | 119 | 0.023 |
| Acute myeloid leukemia | 8 | 61 | 0.024 |
| Human T-cell leukemia virus 1 infection | 15 | 203 | 0.024 |
| PI3K-Akt signaling pathway | 17 | 279 | 0.024 |
| Th17 cell differentiation | 8 | 97 | 0.024 |
| Small cell lung cancer | 9 | 85 | 0.029 |
| Non-small cell lung cancer | 7 | 66 | 0.029 |
| Hippo signaling pathway - multiple | 1 | 27 | 0.042 |
| species | | | |
| Epstein-Barr virus infection | 12 | 175 | 0.042 |
| Viral protein interaction with cytokine | 2 | 71 | 0.042 |
| and cytokine receptor | | | |
| Pathways in cancer | 22 | 449 | 0.044 |
| Alcoholic liver disease | 7 | 117 | 0.045 |
| PPAR signaling pathway | 1 | 56 | 0.049 |

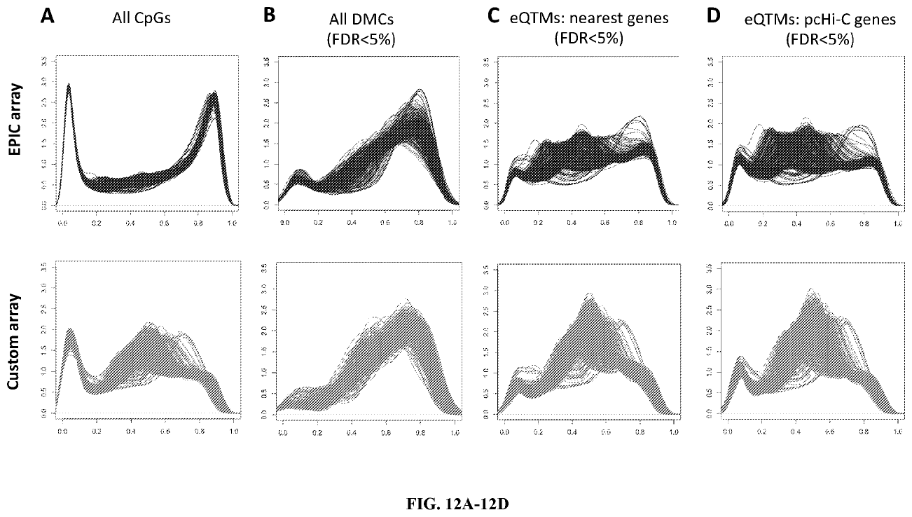

Each CpG-gene pair was tested for correlation between methylation and expression levels to identify expression quantitative methylation (eQTM) CpGs with their nearest and pcHi-C target gene(s) in a linear model that included sex, percent epithelial cells, and three ancestry PCs as covariates (FDR <0.05). Significantly more CpGs on the Custom array were eQTMs with their nearest gene (23%) or pcHi-C target gene (16%) compared to CpGs on the EPIC array (11% and 9% respectively; FET p<2.2×10⁻¹⁶ in both analyses). The filtered EPIC CpGs were also enriched for eQTMs compared to all EPIC CpGs (20% and 12%, respectively; FET p<2.2×10⁻¹⁶ in both analyses). Although there were more AS DMCs that were eQTMs on both arrays, there were still significantly more on the Custom compared to the EPIC array (nearest gene 35% vs. 20% [FET p=0.0019] and pcHi-C 22% vs 15% [FET p=0.0082], respectively) (Table 7). To assess whether the enrichment of eQTMs among DMCs was not due to unaccounted structure in the data, 20 permutations were performed, testing for associations between methylation levels at each Custom array CpG and expression levels of randomly selected genes on different chromosomes (see Methods). Among these permutations, as many eQTMs was never observed as the 23% (all CpGs) and 35% (DMCs only) for nearest gene or the 16% (all CpGs) and 22% (DMCs only) for pcHi-C target genes observed in our data (permutations: all CpGs median 2.6% and range [2.5%-2.8%]; considering only DMCs the median was 2.8% (range 1%-6.7%). The β distributions for DMCs on the Custom and EPIC arrays that were eQTMs were further enriched for IM CpGs and depleted of CpGs at the extremes of the distribution (FIG. 12).

TABLE 7
Enrichment of eQTMs among all CpGs on an
exemplary Custom array and among DMCs.

| | | Percent eQTMs | |
|---|---|---|---|
| | | Nearest | pcHi-C |
| | # of CpGs | Gene | Target Gene |
| EPIC | 789,290 | 11% | 9% |
| Custom | 37,256 | 23% | 16% |
| P-value for difference (FET) | — | <2.2 × 10⁻¹⁶ | <2.2 × 10⁻¹⁶ |

| | | Percent eQTMs | |
|---|---|---|---|
| | | Nearest | pcHi-C |
| | # of DMCs | Gene | Target Gene |
| EPIC | 1,805 | 20% | 15% |
| Custom | 193 | 35% | 22% |
| P-value for difference (FET) | — | 0.0019 | 0.0082 |

Further Insights into the Epigenetic Regulation of Gene Expression at EWAS Loci

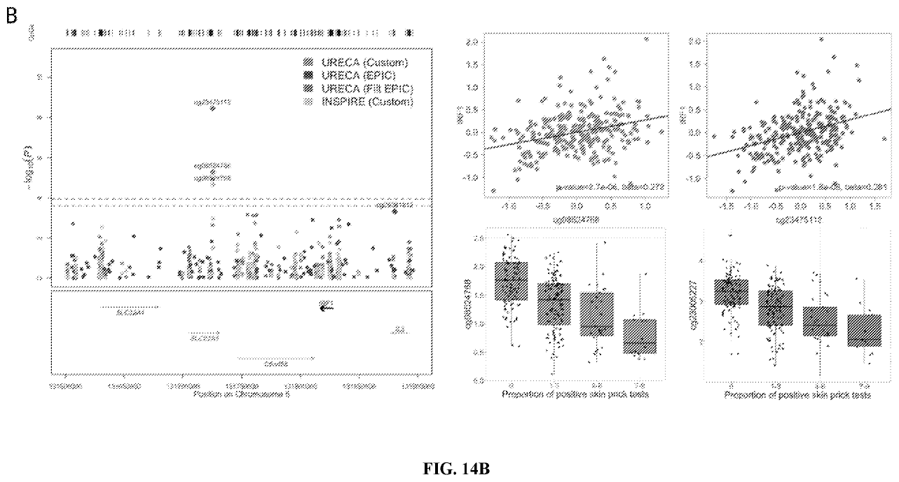

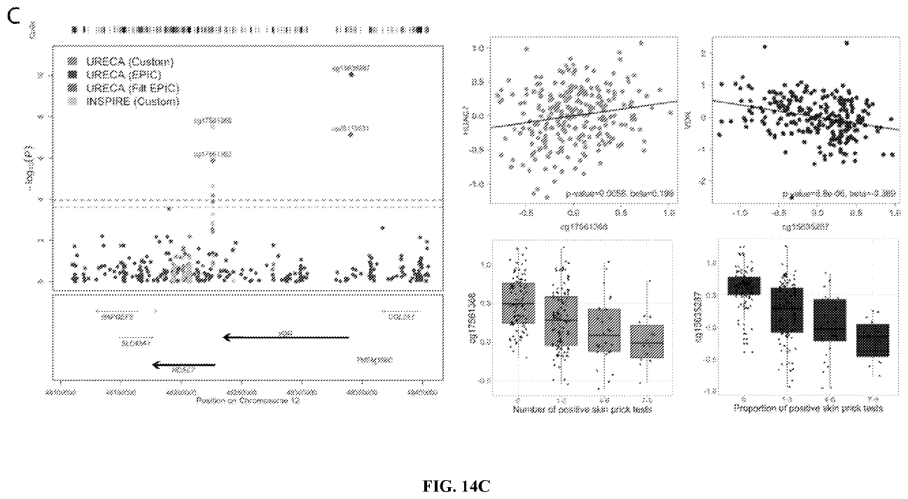

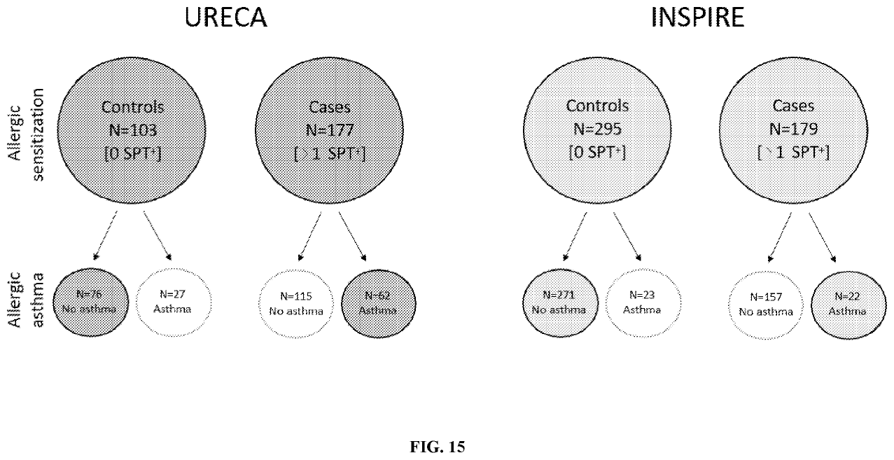

To examine the DMCs and their associations with gene expression more closely, regional association plots for the 10 most significant DMCs in the URECA EWAS using the EPIC and Custom array were generated. The 10 most significant EPIC DMCs were at 10 loci (FIG. 11) Seven were “solitary” with no other DMCs within 500 kb and three had one other DMC within 6.5 kb. Among the solitary DMCs, six were high-value EPIC CpGs and all 10 were reported as DMCs in airway epithelial cells in previous EWAS of asthma or allergic phenotypes. Six of the DMCs were within genes and four were intergenic. CpGs from the Custom array were present in four of these regions, but none were AS DMCs. In contrast, the 10 most significant Custom DMCs were at six loci and only two were solitary (FIG. 15). One solitary DMC in ALOXJ5 was near a high-value and a non-high-value EPIC DMC; the other solitary CpG in the PDE6A gene was also a DMC in the INSPIRE EWAS. The other four loci had spikes of association, often with a combination of DMCs from both arrays in URECA and the Custom array in both URECA and INSPIRE. Eight of the 10 most significant DMCs were within genes; two were intergenic. Three additional regions were selected as examples of patterns of associations (Table 9). The first includes a spike of 11 Custom array DMCs in URECA, 12 in INSPIRE and one high-value EPIC DMC in exons 2 and β of the CISH (Cytokine Inducible SH2-Containing Protein) gene, a member of the SOCs family of negative regulators of cytokine signaling (FIG. 14A). There was one nearby DMC from the EPIC array downstream of CISH. The lead Custom and EPIC URECA DMCs were eQTMs for CISH, with increased methylation associated with decreased gene expression and fewer allergic sensitizations. The EPIC high-value CpG at this locus was identified in previous asthma/allergy EWAS, but this locus has not been associated with asthma or allergic diseases in GWAS. CISH expression is induced by IL-13 in bronchial epithelial cells and macrophages, and has been implicated in eosinophil physiology and eosinophilic inflammation. The studies herein are consistent with these findings and further indicate that increased expression of CISH is associated with increased sensitization to allergens and that the regulation of CISH expression may be epigenetically mediated.

TABLE 9
Annotation information for three selected loci. For each locus, the number of DMCs (URECA and INSPIRE), eQTMs
for the nearest gene and pcHi-C target gene, genic location, primary inclusion criteria (GWAS, EWAS, and/or
DMR), and functional annotation category are shown for each platform (Custom, high-value EPIC, and EPIC).

| | | # DMCs | | | | | | |
|---|---|---|---|---|---|---|---|---|
| | | (# in | | | | | | |
| | | INSPIRE | | | | | | |
| | | that | eQTM | | | | | |
| | | overlap | (Number of DMCs) | | | | | |
| | | with | Nearest | pcHi-C target | | Primary Criteria | | |
| Locus | | URECA) | gene | gene | Location(s) | GWAS | EWAS | DMR |
| CISH | URECA (Custom) | 11 | CISH (11) | RASSF1 (2), MANF (4), | Exons 2 and 3 | 0 | 0 | 11 |
| | | | | ZMYND10 (1), HYAL1 (2) | | | | |
| | INSPIRE (Custom) | 12 (9) | NA | NA | Exon 3 | 0 | 0 | 12 |
| | URECA (high-value EPIC) | 2 | CISH (2) | 0 | Exon 3, intron 1 | 0 | 2 ^{E24, E25} | 1 |
| | URECA (EPIC) | 1 | CISH (1) | HYALI | Intergenic | 0 | 0 | 0 |
| SLC22A5/ | URECA (Custom) | 2 | 0 | IRFI (2), CCNI2 (1), GDF9 | Intron 6 | 2 ^{E9, E10} | 0 | 0 |
| IRF1 | | | | (2), SEPT8 (1), SHROOM1 (1) | | | | |
| | INSPIRE (Custom) | 1 (1) | NA | NA | Intron 6 | 1 ^{E9, E10} | 0 | 0 |
| | URECA (high-value EPIC) | 1 | 0 | IRF1 (1), CCNI2 (1) | Intron 6 | 1 ^{F9, F10} | 1 ^{F24, F25} | 0 |
| | URECA (EPIC) | 0 | 0 | 0 | — | | | |
| HDAC7 | URECA (Custom) | 3 | HDAC7 (1) | SLC48A (1) | Intergenic | 3 ^{E9} | 0 | 3 |
| /VDR | INSPIRE (Custom) | 2 (2) | NA | NA | Intergenic | 2 ^{E9} | 0 | 2 |
| | URECA (high-value EPIC) | 1 | VDR (1) | 0 | Intergenic | 1 ^{E9} | 1^{E25} | 0 |
| | URECA (EPIC) | 1 | VDR (1) | 0 | Intergenic | 1 ^{E9} | 0 | 0 |

| | | Functional Annotations | | | | | |
|---|---|---|---|---|---|---|---|
| | | Open | | | | | |
| | | chromatin | Enhancer | TFBS | TSS | Poised | Active |
| Locus | | (ATAC-seq) | (pcHi-C) | (ENCODE) | (ROADMAP) | enhancer | enhancer |
| CISH | URECA (Custom) | 0 | 11 | 10 | 0 | 11 | 2 |
| | INSPIRE (Custom) | 0 | 12 | 12 | 0 | 12 | 1 |
| | URECA (high-value EPIC) | 0 | 2 | 2 | 1 | 1 | 0 |
| | URECA (EPIC) | 0 | 1 | 1 | 0 | 1 | 1 |
| SLC22A5/ | URECA (Custom) | 2 | 2 | 2 | 0 | 2 | 2 |
| IRF1 | INSPIRE (Custom) | 1 | 1 | 1 | 0 | 1 | 1 |
| | URECA (high-value EPIC) | 1 | 1 | 1 | 0 | 1 | 1 |
| | URECA (EPIC) | | | | | | |
| HDAC7 | URECA (Custom) | 0 | 3 | 3 | 0 | 0 | 3 |
| /VDR | INSPIRE (Custom) | 0 | 2 | 2 | 0 | 0 | 2 |
| | URECA (high-value EPIC) | 0 | 1 | 1 | 0 | 0 | 1 |
| | URECA (EPIC) | 0 | 1 | 1 | 0 | 0 | 1 |

A second locus included one Custom DMC in URECA and INSPIRE, a second Custom DMC in URECA, and one high-value EPIC DMC in an intron of SLC22A5 (Solute Carrier Family 22 Member 5) (FIG. 14B). The lead DMC was a high-value EPIC CpG, which was also identified in prior EWAS in nasal epithelium. None of the DMCs were eQTMs for SLC22A5, but all three were in a region that physically interacted with the promoter of Interferon Response Factor 1 (IRF1), 91 kb away, and were eQTMs for IRF1 (p=2.4×10⁷, beta=0.27 and p=2.7×10⁻⁶, beta=0.27). This region is at a GWAS locus for adult- and childhood-onset asthma and hay fever (Table 9). Genetic variation in the IRF1 gene has been associated with increased expression of pro-inflammatory genes and IL-13 secretion in peripheral blood cells. The studies herein further demonstrate long-range epigenetic regulation of IRF1 expression in airway epithelial cells, with increased methylation levels associated with increased gene expression and decreased numbers of sensitizations.

At a third locus, a spike of three Custom array DMCs was observed in URECA and two in INSPIRE upstream of the HDAC7 (Histone Deacetylase 7) gene (FIG. 14C). One Custom DMC (upstream of HDAC7 and downstream of VDR) was an eQTM for HDAC7. Histone deacetylases (HDACs) have diverse functions, including regulation of inflammatory genes, and impaired barrier function in asthmatics was both induced by HDAC activity and reversed by inhibition of endogenous HDAC. In this study, increased methylation in the HDAC7 gene was associated with increased expression of HDAC7 and fewer sensitizations. At this extended locus, two EPIC DMCs were in an intergenic region 42 kb upstream of the VDR (Vitamin D Receptor) gene. Vitamin D deficiency in childhood has been associated with increased risk for persistent asthma and high dose vitamin D supplementation in pregnancy reduced risk of recurrent wheeze in childhood. Here, increased methylation (EPIC array) near the VDR gene was associated with decreased VDR expression and sensitization to fewer allergens. The HDAC7-VDR region has been identified in GWAS of childhood- and adult-onset asthma. This locus demonstrates the complementarity of the Custom and EPIC arrays in detecting differential methylation relevant to asthma and allergic diseases.

### Evaluating Environmental Effects

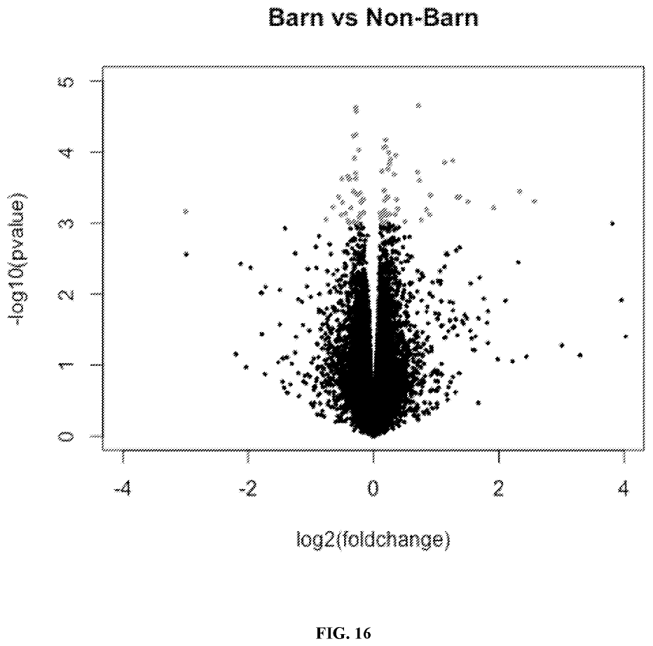

To evaluate whether the CpGs on the arrays described herein are affected by environmental exposures relevant to asthma, array performance was evaluated in blood cell samples obtained from men exposed to cow barns vs. men not exposed to cow barns. The array identified 79 differentially methylated cytosines (DMCs) in men exposed to cow barns compared to men not exposed to cow barns. Results are shown in FIG. 16. This data demonstrates that the arrays described herein find use in methods of identifying subjects that have been exposed to allergens, including environmental allergens, relevant to asthma. For example, the arrays described herein can be used in methods of identifying subjects that have been exposed to environmental allergens known to cause asthma, and thus can be used to identifying subjects having or at risk of having asthma.

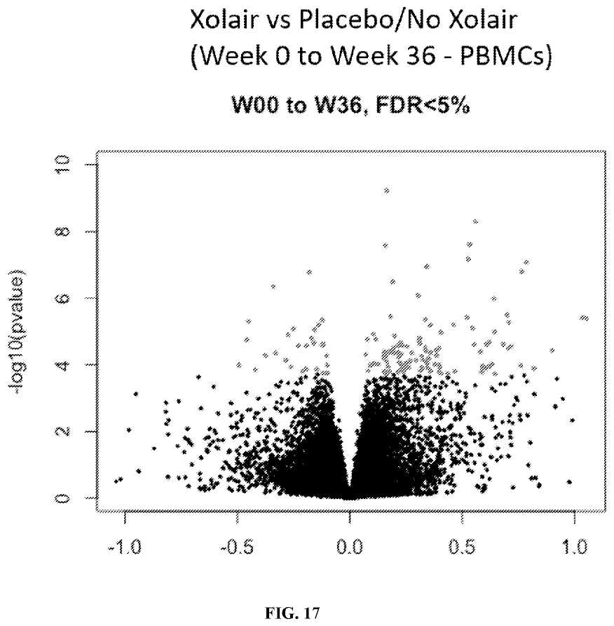

To evaluate whether CpGs on the arrays described herein are affected by allergy treatment, array performance was evaluated in samples obtained from subjects receiving food allergy treatment with Xolair (omalizumab) vs. subjects receiving a placebo treatment for food allergy. The array identified 140 CpGs associated with a differential response to Xolair at 36 weeks following treatment. Results are shown in FIG. 17. This data demonstrates that the arrays described herein find use in methods of evaluating or predicting response to treatment for asthma or allergic disease, including evaluating or predicting response to antibody-based treatment for food allergy.

### II. Discussion

The epigenome plays a critical role in regulating gene expression in a context-specific manner, such as in the presence of environmental exposures or disease states. DNA methylation, for example, is responsive to disease promoting exposures in both in vitro cell models and in ex vivo cells from individuals who are both healthy and with disease. Yet, the DNA methylome has been underexplored and largely limited to the CpGs on commercial arrays. As a result, it is unknown whether the 800,000 or fewer CpGs interrogated in nearly all EWAS studies to date include CpGs most relevant to any specific exposure, disease, or tissue type. Experiments conducted during development of embodiments herein addressed this question by designing a Custom Allergy & Asthma DNA methylation array with high-value CpGs that are not included on the commercial EPIC array (i.e., one embodiment of the arrays within the scope herein). The study design allowed for important observations regarding features of high-value CpGs and to propose a pipeline that can be used to prioritize CpGs from among the more than 28 million in the human genome in future studies.

Although CpGs were not selected based on methylation levels, those that passed through the pipeline were significantly enriched for IM CpGs (β values between 20-80%). The CpGs that were DMCs and eQTMs were further enriched for intermediate methylation levels on both the EPIC and Custom arrays, indicating that this one feature is a signature of high-value, functional CpGs in the genome. IM CpGs are more likely to be tissue-specific, to vary between individuals, and to play important roles in gene regulation. The study further revealed that CpGs with intermediate levels of methylation are also more likely to be associated with AS, an important clinical phenotype that reflects both an immune response to past allergen exposures and a risk factor for the development of asthma and allergic diseases. Indeed, AS DMCs were highly enriched for those associated with allergic asthma. Although the CpGs on the custom array were overall enriched for IM CpGs, AS DMCs were further enriched, with a near complete depletion of very hypomethylated (0-20% methylation) and very hypermethylated (80-100%) CpGs. This important observation was further supported by EWAS results using the EPIC array. Whereas the EPIC array CpGs are enriched for hypomethylated and hypermethylated CpGs in all tissues, the EPIC DMCs were depleted for CpGs at both extremes and enriched for IM CpGs, similar to the DMCs on the custom Allergy&Asthma array. It was demonstrated that the 26,905 CpGs on the EPIC array that passed the filtering pipeline described herein were also enriched for IM CpGs and enriched among AS DMCs: 16.3% of the DMCs in the AS EWAS were among the filtered EPIC CpGs compared to 3.4% of all CpGs on the EPIC array. This data indicate that filtering CpGs on the EPIC array to include just IM CpGs (β=20-80%) in the tissue or cell types in individual studies should enrich for functional CpGs and increase power to detect DMCs associated with exposure or disease outcomes. Using all CpGs on the EPIC, 1,805 DMCs were identified (0.23% of all CpGs). However, when only IM CpGs in the GWAS were included, 2,182 DMCs were identified (0.70% of all CpGs), more than three times the proportion of DMCs among CpGs. Finally, DMCs that were eQTMs were further enriched for IM CpGs, supporting their role in gene regulation.

In addition to the enrichment of IM CpGs on the Custom compared to the EPIC array, the patterns of associations with AS on the two arrays were notable. Whereas DMCs on the EPIC are more sparsely distributed throughout the genome, the DMCs on the Custom commonly form spikes of association, analogous to GWAS peaks. Such regional clustering of DMCs provides confidence in and internal validation of EWAS results, in contrast to the more solitary distribution of DMCs on the EPIC array (FIG. 12). Another distinguishing feature of the DMCs on the Custom array were their enrichment in exons and depletions in intergenic regions compared to DMCs on the EPIC array.

The CpGs included on the custom Allergy&Asthma array were selected based on prior evidence of associations with asthma or allergic diseases using three classes of evidence: within a DMR in our WGBS study, in a previous published EWAS, or at published GWAS loci. Overall, 62% of CpGs on the custom array were selected from DMRs compared to 1.6% of all CpGs on the EPIC array, yet the DMR CpGs were enriched among DMCs on both the custom (77.2%) and EPIC (3.9%) arrays. Because all CpGs on the EPIC were excluded from the Custom array, there were few previous EWAS CpGs on the Custom array, although a modest enrichment of EWAS CpGs among DMCs was still observed on the Custom array (1.9% overall vs. 3.9% of DMCs) and a highly significant enrichment among DMCs on the EPIC array (4.9% overall vs. 21.7% of DMCs). These represent replications of results from previous EWAS of asthma and allergic disease-associated phenotypes. In contrast to these two categories of prior evidence, CpGs at asthma and allergy GWAS loci were not significantly enriched among DMCs from either array and even slightly depleted among the Custom array DMCs. In a WGBS in 25 different cell and tissue types, Elliott et al. (G. Elliott et al., Intermediate DNA methylation is a conserved signature of genome regulation. Nat Commun 6, 6363 (2015) showed that IM CpGs were more likely to be allele-independent than to show allele-specific methylation. Busche et al. (S. Busche et al., Population whole-genome bisulfite sequencing across two tissues highlights the environment as the principal source of human methylome variation. Genome Biol 16, 290 (2015)) conducted a WGBS study in adipose and blood cells from twins and concluded that non-genetic effects were mainly tissue-specific and located in gene regions (consistent with the features of CpGs on the Custom array), whereas CpGs with genetic effects were shared across tissues and more likely to be intergenic (consistent with features of CpGs on the EPIC array). They concluded that non-shared environments accounted for most of the variance in methylation levels. The present findings of a lack of enrichment of DMCs at GWAS loci, but enrichments for IM CpGs and for AS DMCs in exons indicate that inter-individual variation for IM CpGs on the Custom array may be the set of more plastic CpGs in the human genome and more reflective of environmental exposures than of genetic effects. Nonetheless, experiments conducted during development of embodiments herein revealed examples of AS DMCs at important GWAS loci (e.g., IRF1 on chromosome 5, CLEC16A on chromosome 16, and HDAC7/VDR on chromosome 12). Proportionally more CpGs on the custom array are influenced by local genetic variation compared to the EPIC array. Data show that 57% of CpGs on the custom array but only 37% of CpGs on the EPIC are meCpGs, meaning that variation is associated with genotype at least one nearby SNP.

Experiments conducted during development of embodiments herein revealed high-value functional CpGs in airway epithelial cells that are not interrogated on the EPIC array. The development pipeline identified 92,024 high-value CpGs after excluding 26,905 high-value CpGs on the EPIC array. Therefore, among the >28 million CpG sites in the genome, less than 0.5% are likely functional, or high-value, in any particular tissue or disease/environment context. The Custom array (i.e., one embodiment of the present invention), which included about half of the non-EPIC high-value CpGs, revealed that associations with AS and allergic asthma were robust to race/ethnicity, ascertainment, age, and geography.

### III. Methods

### Cohorts Included in WGBS or EWAS Studies

Three longitudinal birth cohort studies were included in these studies. All studies were approved by the Institutional Review Boards of each of the institutions recruiting subjects. The URECA study is an observational birth cohort study initiated in 2005 in Baltimore, Boston, New York City and St. Louis under the NIAID-funded Inner City Asthma Consortium. Either the pregnant mother or the father of their unborn child had a history of asthma, allergic rhinitis, or eczema. Asthma was assessed at age 10 according to a definition that considered symptoms, diagnosis by a health care provider, and measurements of pulmonary function. Skin prick testing was performed at age 10 (255 subjects) or age 7 (25 subjects) and included the following allergens: mouse epithelia, dog epithelia, Dermatophagoides fainae (mite), Dermatophagoides pteronyssinus (mite), cat hair, rat epithelia, American/German cockroach mix, German cockroach, Alternaria tenuis (mold), Aspergillus mix, ragweed mix, tree pollen (oak or birch), Penicillium Notatum/Pennicillium Chrysogenum, and Timothy grass. Nasal brushings were obtained at age 11 years. Twenty African American children were selected for the WGBS studies (10 with asthma and allergic disease [3 females, 7 males], 10 without asthma or allergic disease [3 females, 7 males]), and 280 unrelated children were included in the EWAS using both the Custom and EPIC arrays. The characteristics of the 280 URECA participants are described in Table 4.

The COAST study is an observational birth cohort study initiated in 1998 in Madison, Wisconsin. Pregnant women were recruited in the third trimester of pregnancy if the mother or the father had a history of asthma or allergic diseases. Asthma was assessed beginning at age 6 years. Children were diagnosed with asthma if they fulfilled at least one of the following criteria: (1) physician-diagnosed asthma; (2) frequent albuterol use for coughing or wheezing episodes as prescribed by a physician; (3) use of a prescribed daily controller medication; (4) an implemented step-up plan, including use of albuterol or inhaled corticosteroids during illness as prescribed by a physician; (5) use of prednisone for an asthma exacerbation. Nasal brushings were obtained between ages 18-20 years. Twenty participants of European American ancestry were selected for the WGBS studies (10 with asthma and allergic disease [5 males, 5 females], 10 without asthma or allergic disease [5 males, 5 females]).

The INSPIRE study is an observational birth cohort of healthy infants in central Tennessee (E. K. Larkin et al., Objectives, design and enrollment results from the Infant Susceptibility to Pulmonary Infections and Asthma Following RSV Exposure Study (INSPIRE). BMC Pulm Med 15, 45 (2015). Flocked nasal swabs were obtained at age 5-6 years and samples were stored at −80 C in RNA lysis buffer. RNA and DNA were isolated from the swabs using the Qiagen AllPrep DNA/RNA kit. Subjects were tested for the following thirteen allergens: dog, cat, Dermatophagoides pteronyssinus and Dermatophagoides farinae mix, American/German cockroach, Penicillium Notatum/Penicillium Chrysogenum, Alternaria Tenuis, Cladosporum Herbarum, Aspergillus mix, Ragweed mix, Eastern 6 Tree mix, K-O-T Grass mix, Maple/Box Elder mix, and Weed mix. The characteristics of the 474 unrelated INSPIRE children included in the EWAS are described in Table 4.

### Cohorts Included in the Cross-Tissue Comparison Studies

In addition to studying nasal epithelial cell DNA with the Custom array in URECA, results were included from studies of the Custom array in DNA from nasal lavage cells (n=96), buccal cells (n=96), placenta (n=96) and cord blood (96). The nasal lavage cell DNA was isolated from URECA subjects around age 13 (mean=13.4 years, range=12.8-14.9 years) using the QIAamp DNA Micro Kit. The buccal, placenta and cord blood cell DNA was collected from 96 participants in the VCSIP cohort (11). Placental DNA was extracted from powdered tissue under liquid nitrogen, using the QIAcube for automated nucleic acid extraction. Cord blood DNA was extracted from 200 μL of whole blood using the QIAamp DNA Mini Kit and the QIAcube, and buccal DNA was extracted from buccal swabs following proteinase K digestion using the Maxwell 16 Blood DNA Extraction Kit and the Maxwell 16 Instrument for automated nucleic acid extraction.

### Whole Genome Bisulfite Sequencing and DMR Studies

DNA (250 ng) from participants in the URECA and COAST participants was transferred to the University of Chicago Genomics Facility for bisulfite conversion and sequencing were conducted using the EZ DNA Methylation-Gold Kit library prep and the Swift Biosciences Accel-NGS Methyl-seq DNA library kit, respectively. Samples were sequenced to a minimum of 330 million reads on the Illumina NovaSEQ6000 (S4 flowcell). Adapters were removed from sequence reads using trimgalore (A. Kechin, U. Boyarskikh, A. Kel, M. Filipenko, cutPrimers: A New Tool for Accurate Cutting of Primers from Reads of Targeted Next Generation Sequencing. J Comput Biol 24, 1138-1143 (2017)) prior to mapping the reads using bismark (version 0.18.2) and the hg19 reference assembly. Bismark was further used to remove duplicate reads and call methylation values. Prior to DMR analyses, CpGs were removed if they overlapped with a common SNP (MAF >0.05) in 1000 genomes CEU or YRI populations (C. Genomes Project et al., A global reference for human genetic variation. Nature 526, 68-74 (2015)) or in Blacklisted regions (H. M. Amemiya, A. Kundaje, A. P. Boyle, The ENCODE Blacklist: Identification of Problematic Regions of the Genome. Sci Rep 9, 9354 (2019)). Variant calling from WGBS was performed using the biscuit algorithm, which also identified sample swaps and contamination. The variant calls from WGBS were compared to either array-based genotypes (COAST) or whole genome sequence-based genotypes (URECA). All variant calls matched with an accuracy of >95%, except for one mismatched sample in COAST. The latter sample was excluded from further analysis, leaving 19 samples for analysis (9 allergic asthmatics and 10 non-asthma/non-allergic controls).

Three DMR analyses were conducted in the African American sample (n=20), the European American sample (n=19), and the combined African American and European American sample (n=39). The methylation data were then smoothed using BSmooth and DMRs were called using the bsseq package (version 1.14) (K. D. Hansen, B. Langmead, R. A. Irizarry, BSmooth: from whole genome bisulfite sequencing reads to differentially methylated regions. Genome Biol 13, R83 (2012)). Only CpGs covered by at least 10 reads in 80% of the cases and controls (AA only, EA only, and combined n=39) were included. T-statistics cutoffs were based on 5% quantiles and a maximum gap of 300 bp was required between CpGs to define a cluster, as recommended. DMRs were then filtered to require three or more CpGs per DMR and a minimum of 5% difference in methylation levels between the allergy asthma cases and non-allergy, non-asthmatic controls. The union of DMRs between analyses was assessed using the reduce function from the GenomicRanges β package (version 1.30).

### Selection of CpGs for Custom Array

CpGs were first identified with a high likelihood of being associated with asthma and allergic disease (FIG. 5). In the first step, regions with prior evidence of association with asthma or allergic diseases (atopic dermatitis/eczema, allergic rhinitis/hay fever, and food allergy) were prioritized from three categories of studies. The first category included the 199,473 CpGs within the DMRs from the WGBS. The second category included CpGs from previous DNA methylation studies (EWAS) of asthma or allergic diseases. For this, a literature search was conducted for array-based studies of DNA methylation in asthma and allergic disease. 15 studies were identified that conducted 25 EWAS of asthma- or allergy-related phenotypes, five of which were conducted in respiratory epithelium and 10 in blood)(Table 2). CpG sites for five additional genes from two candidate gene DNA methylation studies of food allergies were also included; FOXP3 and IL4, IL5, IL10 and INFG. In total, 19,057 unique DMCs were identified. The third category included CpGs located within the 140 GWAS loci defined in two recent large studies of adult-onset and childhood-onset asthma (M. Pividori, N. Schoettler, D. L. Nicolae, C. Ober, H. K. Im, Shared and distinct genetic risk factors for childhood-onset and adult-onset asthma: genome-wide and transcriptome-wide studies. Lancet Respir Med 7, 509-522 (2019)) and allergic diseases (asthma, hay fever and eczema) (A. Johansson, M. Rask-Andersen, T. Karlsson, W. E. Ek, Genome-wide association analysis of 350 000 Caucasians from the UK Biobank identifies novel loci for asthma, hay fever and eczema. Hum Mol Genet 28, 4022-4041 (2019)) in UK Biobank subjects (C. Bycroft et al., The UK Biobank resource with deep phenotyping and genomic data. Nature 562, 203-209 (2018)), which included nearly all the loci reported in other GWAS in multi-ancestry populations. In total, the GWAS loci covered a total of 570,350 CpG motifs. CpGs for the MALT1 gene that was significantly associated with peanut allergy in a genome-wide interaction study (A. Winters et al., The MALT1 locus and peanut avoidance in the risk for peanut allergy. J Allergy Clin Immunol 143, 2326-2329 (2019)) were also included. Duplicate CpGs were removed from among the three categories of prior studies, CpGs on the EPIC array, CpGs in ENCODE blacklist regions, and those in which the cytosine nucleotide overlapped with common SNPs (MAF >5%) in 1000 Genomes CEU or YRI populations. A total of 696,225 CpGs remained for consideration in the second step.

To further prioritize the CpGs, in the second step overlap with six functional annotations was considered: 1) ENCODE (E. P. Consortium et al., Expanded encyclopaedias of DNA elements in the human and mouse genomes. Nature 583, 699-710 (2020)) TFBSs from all cell types; 2-4) ROADMAP Epigenetics (C. Roadmap Epigenomics et al., Integrative analysis of 111 reference human epigenomes. Nature 518, 317-330 (2015)) transcriptional start sites, poised enhancers and active enhancers from smooth muscle (E078, E076, E103, E111), epithelial (E055, E056, E059, E061, E058), and blood cells (E062, E034, E045, E044, E043, E039, E041, E042, E040, E037, E048, E038, E047, E029, E050, E032, E046); 5) ATAC-seq in human cultured bronchial epithelial cells exposed to rhinovirus or vehicle from asthmatic and non-asthmatic individuals, and 6) pcHi-C from ex vivo human bronchial epithelial cells (B. A. Helling et al., Altered transcriptional and chromatin responses to rhinovirus in bronchial epithelial cells from adults with asthma. Commun Biol 3, 678 (2020)). It was required that CpGs at DMRs overlapped with at least three functional annotations or prior evidence (GWAS or EWAS), CpGs from previous EWASs overlapped with at least one functional annotation or prior evidence (GWAS or DMR), and CpGs at GWAS loci overlapped with at least four functional annotations or prior evidence (EWAS or DMR). Lastly, all remaining CpGs that were within both a GWAS locus and a DMR were selected.

From the 92,024 resulting high-value CpGs identified, an exemplary array was manufactured (the “Custom array”), containing probes that passed quality control and manufacturing amounting to 53,840 probes targeting 45,891 CpGs.

### Rna-Seq Studies in URECA

Protocols for processing samples for RNA-seq in nasal epithelial cells from the URECA children have been described (M. C. Altman et al., Endotype of allergic asthma with airway obstruction in urban children. J Allergy Clin Immunol 10.1016/j.jaci.2021.02.040 (2021)). Gene expression data were available in 249 of the children with DNA methylation data. In these data, 15,643 genes were detected as expressed.

### Estimation of Genetic Ancestry

Ancestry PCs were estimated in the URECA children using a set of 3,534 SNPs that were genotyped in URECA and in reference panels from the 1000 Genomes Project (1 KG; n=156) (C. Genomes Project et al., A global reference for human genetic variation. Nature 526, 68-74 (2015)) and the Human Genome Diversity Project (HGDP; n=52). European, West African, and East Asian reference samples were randomly selected from CEU (n=52), YRI (n=52), JPT (n=26), and CHB (n=26) samples in the phase 3 1 KG reference panel. Ancestry PCs were calculated PC-Air (M. P. Conomos, M. B. Miller, T. A. Thornton, Robust inference of population structure for ancestry prediction and correction of stratification in the presence of relatedness. Genet Epidemiol 39, 276-293 (2015)).

### Processing Custom and EPIC Array Methylation Data in URECA

DNA methylation was assessed using the Illumina Allergy&Asthma Custom BeadChip or the Illumina Infinium MethylationEPIC BeadChip (Illumina, San Diego, CA) following bisulfite conversion at the University of Chicago Genomics Facility. Methylation data from both arrays were processed using minfi v1.29.1 (M. J. Aryee et al., Minfi: a flexible and comprehensive Bioconductor package for the analysis of Infinium DNA methylation microarrays. Bioinformatics 30, 1363-1369 (2014)).

For the EPIC array, probes that failed (detection P<0.01 in at least 25% of samples), overlapped with known SNPs with MAF of at least 5% in African American or European Americans, mapped to the X or Y chromosomes, overlapped ENCODE blacklist regions, or mapped to multiple locations in a bisulfite-converted genome were removed. Raw probe values were background corrected using preprocessIllumina (bg.correct=“TRUE”, normalize=“no”), and quantile normalization was performed using ENmix (v 1.30.01), followed by SWAN normalization (J. Maksimovic, L. Gordon, A. Oshlack, SWAN: Subset-quantile within array normalization for illumina infinium HumanMethylation450 BeadChips. Genome Biol 13, R44 (2012)). Samples that failed sex checks using the getSex function in minfi were removed. DNA concentration, collection site, array and plate showed batch effects by principal components analysis (PCA). The effects of collection site, array, and plate were removed using ComBat (W. E. Johnson, C. Li, A. Rabinovic, Adjusting batch effects in microarray expression data using empirical Bayes methods. Biostatistics 8, 118-127 (2007)); DNA concentration was removed using linear regression. It was estimated unobserved variation in the data that was not correlated with our phenotype of interest and included these latent factors in the model. The first three ancestry PCs were also included as covariates to capture the effects of admixture in the sample. After QC, we retained 789,290 (91.1%) of CpGs on the EPIC array for analysis.

For an exemplary Custom array provided herein, probes that failed (detection P<0.01 in at least 25% of samples), contained a SNP with MAF of at least 5% in African American or European Americans within β bp of the CpG interrogation site, mapped to the X chromosome, or were missing genomic coordinates were removed. Raw probe values were background corrected using preprocessIllumina (bg.correct=“TRUE”, normalize=“no”), and quantile normalization was performed using ENmix. DNA concentration, collection site, array, and plate showed batch effects by PCA. The effects of site, plate, and chip were removed using ComBat, and DNA concentration was removed using linear regression. It was estimated unobserved variation in the data that was not correlated with the phenotype of interest and included these latent factors in the model. To account for regional differences in the density of Custom array CpGs on the estimation of latent factors, we used the partition.params function in FALCO (https://github.com/chrismckennan) to partition groups of CpGs into independent units (default size=1e5). The first three ancestry PCs were included as covariates to capture the effects of admixture in the sample for analyses in URECA. After QC, 37,256 (98.1%) CpGs were retained for analyses.

For comparison purposes, the CpGs were filtered on the EPIC array through the same pipeline described above for selecting CpGs for the Custom array; we refer to these as “filtered” EPIC CpGs. Of the 789,290 CpGs on the EPIC, 26,905 (3.4%) were included in the filtered dataset.

### Processing Custom Array Methylation Data in INSPIRE

The same pipeline used in URECA was used to process INSPIRE Custom methylation data. Twenty-one probes failed P-value detection and were removed from the analysis. Sex was estimated using the X chromosome CpGs and removed five samples with discrepancies between these classifications and reported sex. DNA concentration, collection year, plate, and array were identified as having batch effects by PCA. The effects of collection year, plate, and array were removed using ComBat, and DNA concentration was removed using linear regression. Because DNA concentration was correlated with other variables, it was also included as a covariate in the model, along with sex, ethnicity and latent factors estimated using the same methods described above for URECA. A total of 37,261 CpGs passed processing QC and were included in the analysis.

### EWAS in the URECA and INSPIRE Cohorts

For each EWAS we used the following models:
URECA: DNAm˜proportion positive SPTs+sex+epithelial cells+AncPCs₁₋₃+LFs₁₋ₙ
INSPIRE: DNAm˜proportion positive SPTs+sex+self-reported ethnicity+DNA concentration+LF_{S1-n}

For the URECA EWAS, 14 and 5 latent variables were included for the Custom (18) and EPIC (19) arrays, respectively, after removing technical variables (Table 8). Because we did not have information on cell proportion or genetic ancestry for INSPIRE, we included sex, self-reported race, DNA concentration, and 21 latent factors as covariates to adjust for cell composition, as well as other unwanted variation in the EWAS. Analyses were performed in R (version 4.1.0) using limma v 3.50.0 (B. Phipson, S. Lee, I. J. Majewski, W. S. Alexander, G. K. Smyth, Robust Hyperparameter Estimation Protects against Hypervariable Genes and Improves Power to Detect Differential Expression. Ann Appl Stat 10, 946-963 (2016); M. E. Ritchie et al., limma powers differential expression analyses for RNA-sequencing and microarray studies. Nucleic Acids Res 43, e47 (2015)). To control the false discovery rate, a q-value threshold of 0.05 was used. The URECA EWAS sample included 19 of the 20 African American children in the WGBS studies and the 96 children shown in the cross-tissue density plots of β distributions (FIG. 3).

TABLE 8
PCA of DNA methylation analysis and identifying batch effects. Significance of correlations between potential confounders
and PCs 1 through 5 for A) quantile normalized methylation data in URECA subjects (Custom), B) final adjusted methylation
data for URECA subjects (Custom), C) quantile normalized methylation data in URECA subjects (EPIC), D) final adjusted
methylation data for URECA subjects (EPIC), E) quantile normalized methylation data in INSPIRE subjects (Custom), and
F) final adjusted methylation data for INSPIRE subjects (Custom). Values less than P = 0.05 are highlighted in red.

| | | | | | | | | | | |
|---|---|---|---|---|---|---|---|---|---|---|
| | | | Study_{—} | DNA_{—} | | % | % | | | |
| PropVar | Plate | Array | Site | Conc | Sex | Ciliated | Squamous | AncPC1 | AncPC2 | AncPC3 |

A. URECA Custom Array, raw

| | | | | | | | | | | | |
|---|---|---|---|---|---|---|---|---|---|---|---|
| PC1 | 0.386 | 0.236 | 0.016 | 0.103 | 1.37E−08 | 0.960 | 3.38E−25 | 0.008 | 0.265 | 0.198 | 0.230 |
| PC2 | 0.133 | 1.17E−90 | 4.31E−72 | 0.690 | 0.355 | 0.223 | 0.946 | 0.884 | 0.697 | 0.924 | 0.677 |
| PC3 | 0.079 | 0.002 | 0.002 | 1.72E−10 | 0.882 | 0.243 | 0.153 | 0.001 | 0.041 | 0.079 | 0.962 |
| PC4 | 0.029 | 0.246 | 0.919 | 1.54E−08 | 7.29E−07 | 0.016 | 0.896 | 0.180 | 0.150 | 0.129 | 0.361 |
| PC5 | 0.019 | 0.754 | 0.845 | 0.388 | 0.484 | 1.63E−07 | 0.243 | 0.862 | 0.186 | 0.117 | 0.893 |

B. URECA Custom Final. The effects of DNA concentration, plate, array, and study site were
removed. Sex, % ciliated cells, ancestry PCs 1-3, and latent factors were included in the model.

| | | | | | | | | | | | |
|---|---|---|---|---|---|---|---|---|---|---|---|
| PC1 | 0.389 | 0.970 | 1.000 | 0.738 | 0.839 | 0.950 | 2.15E−22 | 0.028 | 0.097 | 0.065 | 0.480 |
| PC2 | 0.089 | 0.936 | 0.999 | 0.410 | 0.436 | 0.038 | 0.231 | 0.002 | 0.130 | 0.236 | 0.817 |
| PC3 | 0.060 | 0.907 | 1.000 | 0.968 | 0.181 | 0.327 | 0.825 | 0.604 | 0.584 | 0.443 | 0.708 |
| PC4 | 0.030 | 0.982 | 1.000 | 0.296 | 0.605 | 0.059 | 0.175 | 0.362 | 0.033 | 0.151 | 0.366 |
| PC5 | 0.024 | 0.954 | 1.000 | 0.283 | 0.922 | 8.17E−07 | 0.462 | 0.614 | 0.037 | 0.030 | 0.862 |

C. URERCA EPIC Array, raw

| | | | | | | | | | | | |
|---|---|---|---|---|---|---|---|---|---|---|---|
| PC1 | 0.229 | 0.139 | 0.064 | 0.128 | 3.498.08 | 0.841 | 2.62E−25 | 0.003 | 0.405 | 0.369 | 0.736 |
| PC2 | 0.064 | 0.002 | 0.002 | 3.20E−09 | 0.676 | 0.130 | 0.487 | 0.008 | 0.016 | 0.188 | 0.285 |
| PC3 | 0.021 | 0.280 | 0.176 | 0.966 | 0.826 | 0.935 | 0.247 | 0.774 | 0.089 | 0.163 | 0.773 |
| PC4 | 0.019 | 0.609 | 0.932 | 1.08E−07 | 0.010 | 0.244 | 0.172 | 0.280 | 0.025 | 0.030 | 0.130 |
| PC5 | 0.017 | 0.199 | 0.137 | 0.675 | 0.284 | 0.123 | 0.416 | 0.644 | 0.085 | 0,015 | 0.292 |

Rna-Sequencing and Expression Quantitative Trait Methylation (eQTM) Studies in URECA
Protocols for processing samples for RNA-seq in nasal epithelial cells from the URECA children have been described²¹. Gene expression data were available in 249 of the children with DNA methylation data. In these samples, 15,643 genes were detected as expressed. To test for cis associations between DNA methylation and gene expression (eQTM studies), we used a linear model that included sex, percent epithelial cells, and three ancestry PCs as covariates (FDR <0.05).
Correlations of Methylation Level with Expression of Nearest Gene and Target Promoter Capture Hi-C Gene

Correlations between methylation levels at each CpG with the expression levels of the nearest gene and the pcHi-C target genes (42) were assessed using linear regression in both the full data set (all CpGs that passed QC on each array) and in the DMCs. Comparisons were only assessed for DMCs falling in “capture end regions” (+/−1 kb) that interacted with gene promoters (42). Sex, ancestry PCs and epithelial cell composition were included as covariates in the model. The nearest gene was annotated using the GenomicRanges package v 1.46.1 in β with the gene list from the β biomaRt package v2.50.2 using ensemble GRCh37. A false discovery rate of 5% was used to assess significance.

### Pathway Analysis

Pathway over-representation testing was performed with Advaita Bio's iPathwayGuide using KEGG Pathways (release 100.0+/11-12, November 2021). For both the Custom (N=318) and the EPIC (2,366) arrays, genes that were either the nearest gene or a pcHi-C target gene to a DMC were entered as significant against a background of all nearest genes to CpGs on both arrays that are expressed in NECs (N=14,049).

### Dna Extraction Protocols:

URLCA Nasal brushings were obtained at age 11 years. Total DNA was isolated from brushes stored in RLT Plus lysis buffer. Samples were thawed, vortexed, and then spun to collect the supernatant, which was transferred to fresh tubes. Seventy percent EtOH was used to wash the brushes and original tubes, which was then transferred to the new tubes. The samples were spun through a Qiashredder column (Qiagen) and then extracted using AllPrep DNA/RNA mini kits (Qiagen) with 100 ul elution volumes for DNA following the manufacturer's protocol. Nasal lavage DNA was isolated using the QIAamp DNA Micro Kit. COAST: DNA was extracted from nasal brushings obtained at ages 18-20 years.

INSPIRE: Flocked nasal swabs were obtained at age 5-6 years and samples were stored at −80° C. in RNA lysis buffer. RNA and DNA were isolated from the swabs using the Qiagen AllPrep DNA/RNA kit.
VCSIP: Placental DNA was extracted from powdered tissue under liquid nitrogen, using the QIAcube for automated nucleic acid extraction. Cord blood DNA was extracted from 200 mL of whole blood using the QIAamp DNA Mini Kit and the QfAcube, and buccal DNA was extracted from buccal swabs following proteinase K digestion using the Maxwell 16 Blood DNA Extraction Kit and the Maxwell 16 Instrument for auwomrated nucleic acid extraction.

### DMR Analysis

DMR analyses were conducted in the African American sample (n=20), the European American sample (n=19), and the combined African American and European American sample (n=39). The methylation data were then smoothed using BSmooth (Hansen K D, Langmead B, Irizarry R A. BSmooth: from whole genome bisulfite sequencing reads to differentially methylated regions. Genome Biol. 2012; 13(10):R83) and DMRs were called using the bsseq package (version 1.14)). Only CpGs covered by at least 10 reads in 80% of the cases and controls (AA only, EA only, and combined n=39) were included. T-statistics cutoffs were based on 5% quantiles, and a maximum gap of 300 bp was required between CpGs to define a cluster, as recommended by BSmooth. To maximize the number of DMRs, three or more CpGs per DMR and a minimum of 5% difference in methylation levels between the allergy asthma cases and non-allergy, non-asthmatic controls were required. The union of DMRs between analyses was assessed using the reduce function from the GenomicRanges β package (version 1.30).

### Estimating Genetic Ancestry

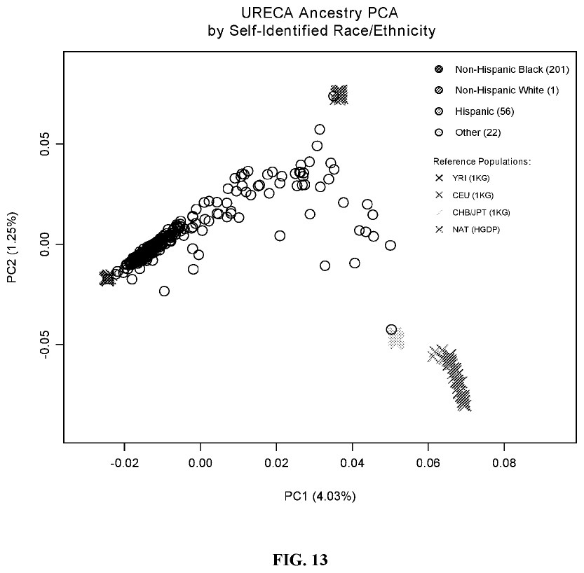

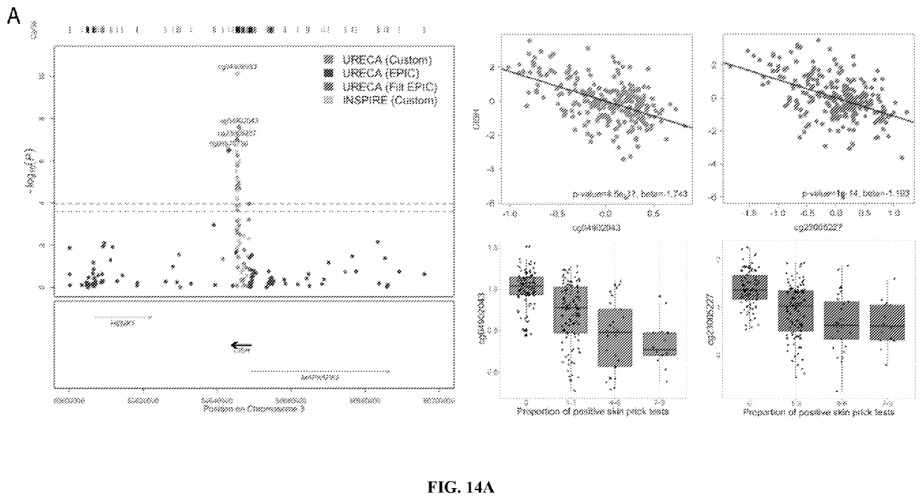

Ancestry PCs were estimated in the URECA children using a set of 3,534 SNPs that were genotyped in URECA and in reference panels from the 1000 Genomes Project (1 KG; n=156) (Genomes Project C, Auton A, Brooks L D, Durbin R M, Garrison E P, Kang H M, et al. A global reference for human genetic variation. Nature. 2015; 526(7571):68-74) and the Human Genome Diversity Project (HGDP; n=52). European, West African, and East Asian reference samples were randomly selected from CEU (n=52), YRI (n=52), JPT (n=26), and CHB (n=26) samples, respectively, in the phase 3 1 KG reference panel. Ancestry PCs were calculated using PC-Air (Conomos M P, Miller M B, Thornton T A. Robust inference of population structure for ancestry prediction and correction of stratification in the presence of relatedness. Genet Epidemiol. 2015; 39(4):276-93). The first two ancestry PCs for the URECA children in our study are shown in FIG. 13.

1. A composition comprising 1,000 or more probe oligonucleotides, each of the 1,000 or more probe oligonucleotides comprising a distinct sequence capable of hybridizing to a CpG site provided in Table A.

2. The composition of claim 1, comprising 10,000 or more probe oligonucleotides comprising a distinct sequence capable of hybridizing to a CpG site provided in Table A.

3. The composition of claim 2, comprising 30,000 or more probe oligonucleotides comprising a distinct sequence capable of hybridizing to a CpG site provided in Table A.

4. The composition of claim 1, wherein the oligonucleotides are deoxyribonucleic acid (DNA) oligonucleotides.

5. A device comprising the composition of one of claims 1-4, wherein the oligonucleotides are displayed on a surface of a substrate.

6. The device of claim 5, wherein the oligonucleotides are tethered to the surface of the substrate.

7. The device of claim 6, wherein the substrate comprises one or more array locations and the oligonucleotides are displayed on a surface within the array location.

8. The device of claim 7, wherein the substrate is a microtiter plate and the array locations are microtiter wells.

9. The device of claim 7, wherein each assay location comprises a plurality of discrete sites for attachment of oligonucleotides to the substrate.

10. The device of claim 9, wherein each discrete site is a bead well within the surface of the substrate.

11. The device of claim 10, wherein the oligonucleotides are tethered to beads and the beads reside within the bead wells on the surface of the substrate.

12. The device of claim 11, wherein each of the probe oligonucleotides are tethered to a separate bead.

13. The device of claim 9, wherein each of said array locations comprises at least 1000 discrete sites per cm².

14. The device of claim 9, wherein each of said array locations comprises at least 1,000,000 discrete sites per cm².

15. A composition comprising 1,000 or more probe oligonucleotides, each of the 1,000 or more probe oligonucleotides comprising a sequence selected from SEQ ID NO:1-SEQ ID NO: 53,840.

16. The composition of claim 15, comprising 10,000 or more probe oligonucleotides, each of the 10,000 or more probe oligonucleotides comprising a sequence selected from SEQ ID NO:1-SEQ ID NO: 53,840.

17. The composition of claim 16, comprising 30,000 or more probe oligonucleotides, each of the 30,000 or more probe oligonucleotides comprising a sequence selected from SEQ ID NO:1-SEQ ID NO: 53,840.

18. A device comprising the composition of any one of claims 15-17, wherein the oligonucleotides are displayed on a surface of a substrate.

19. The device of claim 18, wherein the oligonucleotides are tethered to the surface of the substrate.

20. The device of claim 19, wherein the substrate comprises one or more array locations and the oligonucleotides are displayed on a surface within the array location.

21. The device of claim 20, wherein the substrate is a microtiter plate and the array locations are microtiter wells.

22. The device of claim 21, wherein each array location comprises a plurality of discrete sites for attachment of oligonucleotides to the substrate.

23. The device of claim 22, wherein each discrete site is a bead well within the surface of the substrate.

24. The device of claim 23, wherein the oligonucleotides are tethered to beads and the beads reside within the bead wells on the surface of the substrate.

25. The device of claim 24, wherein each of the probe oligonucleotides are tethered to a separate bead.

26. The device of claim 22, wherein each of said array locations comprises at least 1000 discrete sites per cm².

27. The device of claim 26, wherein each of said array locations comprises at least 1,000,000 discrete sites per cm².

28. A method of detecting the presence of nucleic acid sequences in a sample, comprising:
(a) contacting the composition of any one of claims 1-4, the device of any one of claims 5-14, the composition of any one of claims 15-17, or the device of any one of claims 18-27 with a nucleic acid sample; and
(b) detecting the binding of one or more nucleic acids comprising the nucleic acid sequences to one or more of the probe oligonucleotides of the composition of any one of claims 1-4, the device of any one of claims 5-14, the composition of any one of claims 15-17, or the device of any one of claims 18-27.

29. A method of detecting the methylation status of methylation sites in a nucleic acid in a sample, the method comprising:
(a) treating the sample to differentially modify the nucleic acid at methylated and unmethylated methylation sites to produce a differentially-modified nucleic acid;
(b) amplifying the differentially-modified nucleic acid;
(c) fragmenting the differentially-modified nucleic acid into differentially-modified oligonucleotides;
(d) contacting the device of any one of claims 5-14 or the device of any one of claims 18-27 with the differentially-modified oligonucleotides, and allowing the differentially-modified oligonucleotides to hybridize to the probe oligonucleotides, thereby forming probe/differentially-modified oligonucleotide complexes;
(e) labeling the probe/differentially-modified oligonucleotide complexes in a manner that is specific to whether the differentially-modified oligonucleotide of each complex corresponds to a methylated or unmethylated methylation site;
(f) detecting the labeled probe/differentially-modified oligonucleotide complexes; and
(g) analyzing (1) the type of labeling and (2) the location of the probe/differentially-modified oligonucleotide complexes on the surface.

30. The method of claim 29, wherein amplifying the differentially-modified nucleic acid comprises PCR amplification.

31. The method of claim 29, where treating the sample to differentially modify the nucleic acid at methylated and unmethylated methylation sites comprises exposing the sample to bisulfite that converts unmethylated cytosine to uracil but methylated cytosines are protected from conversion.

32. The method of claim 31, wherein amplifying the differentially-modified nucleic acid converts the uracil generated by bisulfite conversion into thymine.

34. The method of claim 29, wherein fragmenting the differentially-modified nucleic acid comprises site-specific fragmentation of the differentially-modified nucleic acid.

35. The method of claim 34, wherein the site-specific fragmentation is by restriction endonuclease.

36. The method of claim 29, wherein fragmenting the differentially-modified nucleic acid comprises random fragmentation of the differentially-modified nucleic acid.

37. The method of claim 36, wherein the random fragmentation comprises chemical, enzymatic, and/or mechanical fragmentation.

38. The method of claim 29, further comprising a step of isolating the differentially-modified nucleic acid and/or differentially-modified oligonucleotides from reagents for amplification and/or fragmentation.

39. The method of claim 29, wherein labeling the probe/differentially-modified oligonucleotide complexes in a manner that is specific to whether the differentially-modified oligonucleotide of each complex comprises performing a single nucleotide extension reaction with labeled nucleotides.

40. The method of claim 39, wherein the labeled nucleotides comprise happens.

41. The method of claim 40, further comprising contacting probe/differentially-modified oligonucleotide complexes following the single nucleotide extension with antibodies capable of binding to the haptens, wherein the antibodies comprise detectable labels.

42. The method of claim 39, wherein the labeled nucleotides comprise detectable labels.

43. The method of claim 42, wherein the detectable labels comprise fluorescent labels.

44. The method of one of claims 28-43, wherein the nucleic acid sample comprising genomic DNA.

45. The method of claim 44, wherein the genomic DNA is human genomic DNA.

46. The method of claim 45, wherein the human genomic DNA is obtained from airway epithelial cells.

47. The method of claim 46, where the cell are obtained from a subject having or suspected of having asthma or allergies.
